SEQUENCE LISTING (119 sequences)

SEQ ID NO: 1 - protein, 268 aa, synthetic construct
  REGION 1..268: note = synthetic amino acid sequence
MAEVQLLESGGGLVQPGGSLRLSCAASGFTFSSYAMSWVRQAPGKGLEWVSYIASGGDTTNYADSVKGRFTISRDNSKNTLYLQMNSLRAEDTAVYYCAKGASAFDYWGQGTLVTVSSGGGGSGGGGSGGGGSTDIQMTQSPSSLSASVGDRVTITCRASQSISSYLNWYQQKPGKAPKLLIYAASYLQSGVPSRFSGSGSGTDFTLTISSLQPEDFATYYCQQSSNDPYTFGQGTKVEIKRAAAHHHHHHGAAEQKLISEEDLNGAA

SEQ ID NO: 2 - protein, 281 aa, synthetic construct
  REGION 1..281: note = synthetic amino acid sequence
MAEVQLLESGGGLVQPGGSLRLSCAASGFTFSSYAMSWVRQAPGKGLEWVSNISSAGKGLEWVSSIDDSGASTYYADSVKGRFTISRDNSKNTLYLQMNSLRAEDTAVYYCAKDSASFDYWGQGTLVTVSSGGGGSGGGGSGGGGSTDIQMTQSPSSLSASVGDRVTITCRASQSISSYLNWYQQKPGKAPKLLIYTASSLQSGVPSRFSGSGSGTDFTLTISSLQPEDFATYYCQQSAASPSTFGQGTKVEIKRAAAHHHHHHGAAEQKLISEEDLNGAA

SEQ ID NO: 3 - protein, 268 aa, synthetic construct
  REGION 1..268: note = synthetic amino acid sequence
MAEVQLLESGGGLVQPGGSLRLSCAASGFTFSSYPMSWVRQAPGKGLEWVSAIQHTGAPTTYADSVKGRFTISRDNSKNTLYLQMNSLRAEDTAVYYCAKAFPPFDYWGQGTLVTVSSGGGGSGGGGSGGGGSTDIQMTQSPSSLSASVGDRVTITCRASQSISSYLNWYQQKPGKAPKLLIYSASSLQSGVPSRFSGSGSGTDFTLTISSLQPEDFATYYCQQRETGPKAFGQGTKVEIKRAAAHHHHHHGAAEQKLISEEDLNGAA

SEQ ID NO: 4 - protein, 292 aa, synthetic construct
  REGION 1..292: note = Synthetic amino acid sequence
ETVIMKYLLPTAAAGLLLLAAQPAMAEVQLLESGGGLVQPGGSLRLSCAASGFTFSSYAMSWVRQAPGKGLEWVSSIQPEGRRTAYVDSVKGRFTISRDNSKNTLYLQMNSLRAEDTAVYYCAKPPERFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKRAAAHHHHHHGAAEQKLISEEDLNGAA

SEQ ID NO: 5 - protein, 268 aa, synthetic construct
  REGION 1..268: note = Synthetic amino acid sequence
MAEVQLLESGGGLVQPGGSLRLSCAASGFTFSSYAMSWVRQAPGKGLEWVSSIGQKGGGTQYADSVKGRFTISRDNSKNTLYLQMNSLRAEDTAVYYCAKHFENFDYWGQGTLVTVSSGGGGSGGGGSGGGGSTDIQMTQSPSSLSASVGDRVTITCRASQSISSYLNWYQQKPGKAPKLLIYAASHLQSGVPSRFSGSGSGTDFTLTISSLQPEDFATYYCQQTRRPPSTFGQGTKVEIKRAAAHHHHHHGAAEQKLISEEDLNGAA

SEQ ID NO: 6 - protein, 286 aa, synthetic construct
  REGION 1..286: note = Synthetic amino acid sequence
MKYLLPTAAAGLLLLAAQPAMAQVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCARGGDYGSGDYWGQGTLVTVSSGGGGSGGGGSGGGGSNFMLTQDPAVSVALGQTVRITCQGDSLRSYYASWYQQKPGQAPLLVIYGKNIRPSGIPDRFSGSSSGNSASLTITGAQAEDEADYYCHSRDSSGTHLRVFGGGTKVTVLGAAAHHHHHHGAAEQKLISEED

SEQ ID NO: 7 - protein, 276 aa, synthetic construct
  REGION 1..276: note = Synthetic amino acid sequence
MAQVQLQESGGGVVQPGRSLRLSCAASGFTFSTSGMHWVRQAPGKGLEWVAFILHDGSDKYYADSVKGRFTISRDNSKNTLYLQMNSLRAEDTAIYYCAKSQRELLGAEYLQNWGQGTLVTVSSGGGGSGGGGSGGGGSQSALTQPASVSGSPGQSITISCTGTSSDVGGYKYVSWYQQHPGKAPKVMIYDVSNRPSGVSNRFSGSKSGNTASLTISGLQAEDEADYYCSSYTSSSTLVFGGGTKVTVLGAAAHHHHHHGAAEQKLISEEDLNGAA

SEQ ID NO: 8 - protein, 244 aa, synthetic construct
  REGION 1..244: note = synthetic amino acid sequence
MAEVQLLESGGGLVQPGGSLRLSCAASGFTFSSYAMSWVRQAPGKGLEWVSDIGGDGYNTSYADSVKGRFTISRDNSKNTLYLQMNSLRAEDTAVYYCAKSYTAFDYWGQGTLVTVSSGGGGSGGGGSGGGGSTDIQMTQSPSSLSASVGDRVTITCRASQSISSYLNWYQQKPGKAPKLLIYSASGLQSGVPSRFSGSGSGTDFTLTISSLQPEDFATYYCQQDTNGPSTFGQGTKVEIKRAA

SEQ ID NO: 9 - protein, 144 aa, synthetic construct
  REGION 1..144: note = synthetic amino acid sequence
MAEVFDYWGQGTLVTVSSGGGGSGGGGSGGGGSTDIQMTQSPSSLSASVGDRVTITCRASQSISSYLNWYQQKPGKAPKLLIYYASTLQSGVPSRFSGSGSGTDFTLTISSLQPEDFATYYCQQNYNSPYTFGQGTKVEIKRAA

SEQ ID NO: 10 - protein, 244 aa, synthetic construct
  REGION 1..244: note = synthetic amino acid sequence
MAEVQLLESGGGLVQPGGSLRLSCAASGFTFSSYAMSWVRQAPGKGLEWVSTINNSGTSTNYADSVKGRFTISRDNSKNTLYLQMNSLRAEDTAVYYCAKSTNYFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQNAADPTTFGQGTKVEIKRAA

SEQ ID NO: 11 - protein, 244 aa, synthetic construct
  REGION 1..244: note = synthetic amino acid sequence
MAEVQLLESGGGLVQPGGSLRLSCAASGFTFSSYAMSWVRQAPGKGLEWVSSIASSGDDTNYADSVKGRFTISRDNSKNTLYLQMNSLRAEDTAVYYCAKTASSFDYWGQGTLVTVSSGGGGSGGGGSGGGGSTDIQMTQSPSSLSASVGDRVTITCRASQSISSYLNWYQQKPGKAPKLLIYSASSLQSGVPSRFSGSGSGTDFTLTISSLQPEDFATYYCQQTNGNPNTFGQGTKVEIKRAA

SEQ ID NO: 12 - protein, 170 aa, synthetic construct
  REGION 1..170: note = synthetic amino acid sequence
MAEVQLLESGGGLVQPGGSLRLSCAASGFTFSSYAMSWVRQAPGKGLEWVSTIRHAGQSTDIQMTQSPSSLSASVGDRVTITCRASQSISSYLNWYQQKPGKAPKLLIYMASRLQSGVPSRFSGSGSGTDFTLTISSLQPEDFATYYCQQQRTKPPTFGQGTKVEIKRAA

SEQ ID NO: 13 - protein, 244 aa, synthetic construct
  REGION 1..244: note = synthetic amino acid sequence
MAEVQLLESGGGLVQPGGSLRLSCAASGFTFSSYAMSWVRQAPGKGLEWVSAIGASGNATAYADSVKGRFTISRDNSKNTLYLQMNSLRAEDTAVYYCAKSTTDFDYWGQGTLVTVSSGGGGSGGGGSGGGGSTDIQMTQSPSSLSASVGDRVTITCRASQSISSYLNWYQQKPGKAPKLLIYNASGLQSGVPSRFSGSGSGTDFTLTISSLQPEDFATYYCQQAANYPTTFGQGTKVEIKRAA

SEQ ID NO: 14 - protein, 244 aa, synthetic construct
  REGION 1..244: note = synthetic amino acid sequence
MAEVQLLESGGGLVQPGGSLRLSCAASGFTFSSYAMSWVRQAPGKGLEWVSSIYSDGGATSYADSVKGRFTISRDNSKNTLYLQMNSLRAEDTAVYYCAKATATFDYWGQGTLVTVSSGGGGSGGGGSGGGGSTDIQMTQSPSSLSASVGDRVTITCRASQSISSYLNWYQQKPGKAPKLLIYSASALQSGVPSRFSGSGSGTDFTLTISSLQPEDFATYYCQQSSTSPSTFGQGTKVEIKRAA

SEQ ID NO: 15 - protein, 244 aa, synthetic construct
  REGION 1..244: note = synthetic amino acid sequence
MAEVQLLESGGGLVQPGGSLRLSCAASGFTFSSYAMSWVRQAPGKGLEWVSNIAGNGSYTYYADSVKGRFTISRDNSKNTLYLQMNSLRAEDTAVYYCAKDDAAFDYWGQGTLVTVSSGGGGSGGGGSGGGGSTDIQMTQSPSSLSASVGDRVTITCRASQSISSYLNWYQQKPGKAPKLLIYSASYLQSGVPSRFSGSGSGTDFTLTISSLQPEDFATYYCQQSATTPNTFGQGTKVEIKRAA

SEQ ID NO: 16 - protein, 283 aa, synthetic construct
  REGION 1..283: note = synthetic amino acid sequence
MKYLLPTAAAGLLLLAAQPAMAEVQLLESGGGLVQPGGSLRLSCAASGFTFSSYAMSWVRQAPGKGLEWVSAISSNGSGTQYADSVKGRFTISRDNSKNTLYLQMNSLRAEDTAVYYCAKYGSTFDYWGQGTLVTVSSGGGGSGGGGSGGGGSTDIQMTQSPSSLSASVGDRVTITCRASQSISSYLNWYQQKPGKAPKLLIYAASHLQSGVPSRFSGSGSGTDFTLTISSLQPEDFATYYCQQSDDAPTTFGQGTKVEIKRAAAHHHHHHGAAEQKLISEED

SEQ ID NO: 17 - protein, 263 aa, synthetic construct
  REGION 1..263: note = synthetic amino acid sequence
MAEVQLLESGGGLVQPGGSLRLSCAASGFTFSSYAMSWVRQAPGKGLEWVSAIASAGCTTQYADSVKGRFTISRDNSKNTLYLQMNSLRAEDTAVYYCAKCNATFDYWGQGTLVTVSSGGGGSGGGGSGGGGSTDIQMTQSPSSLSASVGDRVTITCRASQSISIDLNWYQQKPGKAPKLLIYAASHLQSGVPSRFSGSGSGTDFTLTISSLQPEDFATYYCQQSADSPYTFGQGTKVEIKRAAAHHHHHHGAAEQKLISEED

SEQ ID NO: 18 - protein, 263 aa, synthetic construct
  REGION 1..263: note = synthetic amino acid sequence
MAEVQLLESGGGLVQPGGSLRLSCAASGFTFSSYAMSWVRQAPGKGLEWVSSISAAGDYTTYADSVKGRFTISRDNSKNTLYLQMNSLRAEDTAVYYCAKDSTSFDYWGQGTLVTVSSGGGGSGGGGSGGGGSTDIQMTQSPSSLSASVGDRVTITCRASQSISSYLNWYQQKPGKAPKLLIYAASNLQSGVPSRFSGSGSGTDFTLTISSLQPEDFATYYCQQTAAYPTTFGQGTKVEIKRAAAHHHHHHGAAEQKLISEED

SEQ ID NO: 19 - protein, 263 aa, synthetic construct
  REGION 1..263: note = synthetic amino acid sequence
MAEVQLLESGGGLVQPGGSLRLSCAASGFTFSSYAMSWVRQAPGKGLEWVSNITAAGSDTYYADSVKGRFTISRDNSKNTLYLQMNSLRAEDTAVYYCAKNSTYFDYWGQGTLVTVSSGGGGSGGGGSGGGGSTDIQMTQSPSSLSASVGDRVTITCRASQSISSYLNWYQQKPGKAPKLLIYNASDLQSGVPSRFSGSGSGTDFTLTISSLQPEDFATYYCQQASGDPDTFGQGTKVEIKRAAAHHHHHHGAAEQKLISEED

SEQ ID NO: 20 - protein, 263 aa, synthetic construct
  REGION 1..263: note = synthetic amino acid sequence
MAEVQLLESGGGLVQPGGSLRLSCAASGFTFSSYAMSWVRQAPGKGLEWVSDISGNGGATNYADSVKGRFTISRDNSKNTLYLQMNSLRAEDTAVYYCAKATTGFDYWGQGTLVTVSSGGGGSGGGGSGGGGSTDIQMTQSPSSLSASVGDRVTITCRASQSISSYLNWYQQKPGKAPKLLIYNASYLQSGVPSRFSGSGSGTDFTLTISSLQPEDFATYYCQQSSTTPSTFGQGTKVEIKRAAAHHHHHHGAAEQKLISEED

SEQ ID NO: 21 - protein, 11 aa, synthetic construct
  REGION 1..11: note = complementary determining region
RASQSISSYLN

SEQ ID NO: 22 - protein, 11 aa, synthetic construct
  REGION 1..11: note = synthetic amino acid sequence
QGDSLRSYYAS

SEQ ID NO: 23 - protein, 14 aa, synthetic construct
  REGION 1..14: note = synthetic amino acid sequence
TGTSSDVGGYKYVS

SEQ ID NO: 24 - protein, 7 aa, synthetic construct
  REGION 1..7: note = synthetic amino acid sequence
AASYLQS

SEQ ID NO: 25 - protein, 7 aa, synthetic construct
  REGION 1..7: note = synthetic amino acid sequence
TASSLQS

SEQ ID NO: 26 - protein, 7 aa, synthetic construct
  REGION 1..7: note = synthetic amino acid sequence
SASSLQS

SEQ ID NO: 27 - protein, 7 aa, synthetic construct
  REGION 1..7: note = synthetic amino acid sequence
AASSLQS

SEQ ID NO: 28 - protein, 7 aa, synthetic construct
  REGION 1..7: note = synthetic amino acid sequence
AASHLQS

SEQ ID NO: 29 - protein, 7 aa, synthetic construct
  REGION 1..7: note = synthetic amino acid sequence
GKNIRPS

SEQ ID NO: 30 - protein, 7 aa, synthetic construct
  REGION 1..7: note = synthetic amino acid sequence
DVSNRPS

SEQ ID NO: 31 - protein, 7 aa, synthetic construct
  REGION 1..7: note = synthetic amino acid sequence
SASGLQS

SEQ ID NO: 32 - protein, 7 aa, synthetic construct
  REGION 1..7: note = synthetic amino acid sequence
YASTLQS

SEQ ID NO: 33 - protein, 7 aa, synthetic construct
  REGION 1..7: note = synthetic amino acid sequence
MASRLQS

SEQ ID NO: 34 - protein, 7 aa, synthetic construct
  REGION 1..7: note = synthetic amino acid sequence
NASGLQS

SEQ ID NO: 35 - protein, 7 aa, synthetic construct
  REGION 1..7: note = synthetic amino acid sequence
SASALQS

SEQ ID NO: 36 - protein, 7 aa, synthetic construct
  REGION 1..7: note = synthetic amino acid sequence
SASYLQS

SEQ ID NO: 37 - protein, 11 aa, synthetic construct
  REGION 1..11: note = synthetic amino acid sequence
RASQSISIDLN

SEQ ID NO: 38 - protein, 7 aa, synthetic construct
  REGION 1..7: note = synthetic amino acid sequence
AASNLQS

SEQ ID NO: 39 - protein, 7 aa, synthetic construct
  REGION 1..7: note = synthetic amino acid sequence
NASDLQS

SEQ ID NO: 40 - protein, 7 aa, synthetic construct
  REGION 1..7: note = synthetic amino acid sequence
NASYLQS

SEQ ID NO: 41 - protein, 9 aa, synthetic construct
  REGION 1..9: note = synthetic amino acid sequence
QQSSNDPYT

SEQ ID NO: 42 - protein, 9 aa, synthetic construct
  REGION 1..9: note = synthetic amino acid sequence
QQSAASPST

SEQ ID NO: 43 - protein, 9 aa, synthetic construct
  REGION 1..9: note = synthetic amino acid sequence
QQRETGPKA

SEQ ID NO: 44 - protein, 9 aa, synthetic construct
  REGION 1..9: note = synthetic amino acid sequence
QQSYSTPNT

SEQ ID NO: 45 - protein, 9 aa, synthetic construct
  REGION 1..9: note = synthetic amino acid sequence
QQTRRPPST

SEQ ID NO: 46 - protein, 12 aa, synthetic construct
  REGION 1..12: note = synthetic amino acid sequence
HSRDSSGTHLRV

SEQ ID NO: 47 - protein, 10 aa, synthetic construct
  REGION 1..10: note = synthetic amino acid sequence
SSYTSSSTLV

SEQ ID NO: 48 - protein, 9 aa, synthetic construct
  REGION 1..9: note = synthetic amino acid sequence
QQDTNGPST

SEQ ID NO: 49 - protein, 9 aa, synthetic construct
  REGION 1..9: note = synthetic amino acid sequence
QQNYNSPYT

SEQ ID NO: 50 - protein, 9 aa, synthetic construct
  REGION 1..9: note = synthetic amino acid sequence
QQNAADPTT

SEQ ID NO: 51 - protein, 9 aa, synthetic construct
  REGION 1..9: note = synthetic amino acid sequence
QQTNGNPNT

SEQ ID NO: 52 - protein, 9 aa, synthetic construct
  REGION 1..9: note = synthetic amino acid sequence
QQQRTKPPT

SEQ ID NO: 53 - protein, 9 aa, synthetic construct
  REGION 1..9: note = synthetic amino acid sequence
QQAANYPTT

SEQ ID NO: 54 - protein, 9 aa, synthetic construct
  REGION 1..9: note = synthetic amino acid sequence
QQSSTSPST

SEQ ID NO: 55 - protein, 9 aa, synthetic construct
  REGION 1..9: note = synthetic amino acid sequence
QQSATTPNT

SEQ ID NO: 56 - protein, 9 aa, synthetic construct
  REGION 1..9: note = synthetic amino acid sequence
QQSDDAPTT

SEQ ID NO: 57 - protein, 9 aa, synthetic construct
  REGION 1..9: note = synthetic amino acid sequence
QQSADSPYT

SEQ ID NO: 58 - protein, 9 aa, synthetic construct
  REGION 1..9: note = synthetic amino acid sequence
QQTAAYPTT

SEQ ID NO: 59 - protein, 9 aa, synthetic construct
  REGION 1..9: note = synthetic amino acid sequence
QQASGDPDT

SEQ ID NO: 60 - protein, 9 aa, synthetic construct
  REGION 1..9: note = synthetic amino acid sequence
QQSSTTPST

SEQ ID NO: 61 - protein, 5 aa, synthetic construct
  REGION 1..5: note = synthetic amino acid sequence
SYAMS

SEQ ID NO: 62 - protein, 5 aa, synthetic construct
  REGION 1..5: note = synthetic amino acid sequence
SYPMS

SEQ ID NO: 63 - protein, 5 aa, synthetic construct
  REGION 1..5: note = synthetic amino acid sequence
TSGMH

SEQ ID NO: 64 - protein, 17 aa, synthetic construct
  REGION 1..17: note = synthetic amino acid sequence
YIASGGDTTNYADSVKG

SEQ ID NO: 65 - protein, 30 aa, synthetic construct
  REGION 1..30: note = synthetic amino acid sequence
NISSAGKGLEWVSSIDDSGASTYYADSVKG

SEQ ID NO: 66 - protein, 17 aa, synthetic construct
  REGION 1..17: note = synthetic amino acid sequence
AIQHTGAPTTYADSVKG

SEQ ID NO: 67 - protein, 17 aa, synthetic construct
  REGION 1..17: note = synthetic amino acid sequence
SIQPEGRRTAYVDSVKG

SEQ ID NO: 68 - protein, 17 aa, synthetic construct
  REGION 1..17: note = synthetic amino acid sequence
SIGQKGGGTQYADSVKG

SEQ ID NO: 69 - protein, 17 aa, synthetic construct
  REGION 1..17: note = synthetic amino acid sequence
AISGSGGSTYYADSVKG

SEQ ID NO: 70 - protein, 17 aa, synthetic construct
  REGION 1..17: note = synthetic amino acid sequence
FILHDGSDKYYADSVKG

SEQ ID NO: 71 - protein, 17 aa, synthetic construct
  REGION 1..17: note = synthetic amino acid sequence
DIGGDGYNTSYADSVKG

SEQ ID NO: 72 - protein, 17 aa, synthetic construct
  REGION 1..17: note = synthetic amino acid sequence
TINNSGTSTNYADSVKG

SEQ ID NO: 73 - protein, 17 aa, synthetic construct
  REGION 1..17: note = synthetic amino acid sequence
SIASSGDDTNYADSVKG

SEQ ID NO: 74 - protein, 7 aa, synthetic construct
  REGION 1..7: note = synthetic amino acid sequence
TIRHAGQ

SEQ ID NO: 75 - protein, 17 aa, synthetic construct
  REGION 1..17: note = synthetic amino acid sequence
AIGASGNATAYADSVKG

SEQ ID NO: 76 - protein, 17 aa, synthetic construct
  REGION 1..17: note = synthetic amino acid sequence
SIYSDGGATSYADSVKG

SEQ ID NO: 77 - protein, 17 aa, synthetic construct
  REGION 1..17: note = synthetic amino acid sequence
NIAGNGSYTYYADSVKG

SEQ ID NO: 78 - protein, 17 aa, synthetic construct
  REGION 1..17: note = synthetic amino acid sequence
AISSNGSGTQYADSVKG

SEQ ID NO: 79 - protein, 17 aa, synthetic construct
  REGION 1..17: note = synthetic amino acid sequence
AIASAGCTTQYADSVKG

SEQ ID NO: 80 - protein, 17 aa, synthetic construct
  REGION 1..17: note = synthetic amino acid sequence
SISAAGDYTTYADSVKG

SEQ ID NO: 81 - protein, 17 aa, synthetic construct
  REGION 1..17: note = synthetic amino acid sequence
NITAAGSDTYYADSVKG

SEQ ID NO: 82 - protein, 17 aa, synthetic construct
  REGION 1..17: note = synthetic amino acid sequence
DISGNGGATNYADSVKG

SEQ ID NO: 83 - protein, 7 aa, synthetic construct
  REGION 1..7: note = synthetic amino acid sequence
GASAFDY

SEQ ID NO: 84 - protein, 7 aa, synthetic construct
  REGION 1..7: note = synthetic amino acid sequence
DSASFDY

SEQ ID NO: 85 - protein, 7 aa, synthetic construct
  REGION 1..7: note = synthetic amino acid sequence
AFPPFDY

SEQ ID NO: 86 - protein, 7 aa, synthetic construct
  REGION 1..7: note = synthetic amino acid sequence
PPERFDY

SEQ ID NO: 87 - protein, 7 aa, synthetic construct
  REGION 1..7: note = synthetic amino acid sequence
HFENFDY

SEQ ID NO: 88 - protein, 9 aa, synthetic construct
  REGION 1..9: note = synthetic amino acid sequence
GGDYGSGDY

SEQ ID NO: 89 - protein, 13 aa, synthetic construct
  REGION 1..13: note = synthetic amino acid sequence
SQRELLGAEYLQN

SEQ ID NO: 90 - protein, 7 aa, synthetic construct
  REGION 1..7: note = synthetic amino acid sequence
SYTAFDY

SEQ ID NO: 91 - protein, 7 aa, synthetic construct
  REGION 1..7: note = synthetic amino acid sequence
STNYFDY

SEQ ID NO: 92 - protein, 7 aa, synthetic construct
  REGION 1..7: note = synthetic amino acid sequence
TASSFDY

SEQ ID NO: 93 - protein, 7 aa, synthetic construct
  REGION 1..7: note = synthetic amino acid sequence
STTDFDY

SEQ ID NO: 94 - protein, 7 aa, synthetic construct
  REGION 1..7: note = synthetic amino acid sequence
ATATFDY

SEQ ID NO: 95 - protein, 7 aa, synthetic construct
  REGION 1..7: note = synthetic amino acid sequence
DDAAFDY

SEQ ID NO: 96 - protein, 7 aa, synthetic construct
  REGION 1..7: note = synthetic amino acid sequence
YGSTFDY

SEQ ID NO: 97 - protein, 7 aa, synthetic construct
  REGION 1..7: note = synthetic amino acid sequence
CNATFDY

SEQ ID NO: 98 - protein, 7 aa, synthetic construct
  REGION 1..7: note = synthetic amino acid sequence
DSTSFDY

SEQ ID NO: 99 - protein, 7 aa, synthetic construct
  REGION 1..7: note = synthetic amino acid sequence
NSTYFDY

SEQ ID NO: 100 - protein, 7 aa, synthetic construct
  REGION 1..7: note = synthetic amino acid sequence
ATTGFDY

SEQ ID NO: 101 - protein, 7 aa, synthetic construct
  REGION 1..7: note = synthetic amino acid sequence
GFTFSSY

SEQ ID NO: 102 - protein, 7 aa, synthetic construct
  REGION 1..7: note = synthetic amino acid sequence
GFTFSTS

SEQ ID NO: 103 - protein, 6 aa, synthetic construct
  REGION 1..6: note = synthetic amino acid sequence
ASGGDT

SEQ ID NO: 104 - protein, 6 aa, synthetic construct
  REGION 1..6: note = synthetic amino acid sequence
QHTGAP

SEQ ID NO: 105 - protein, 6 aa, synthetic construct
  REGION 1..6: note = synthetic amino acid sequence
QPEGRR

SEQ ID NO: 106 - protein, 6 aa, synthetic construct
  REGION 1..6: note = synthetic amino acid sequence
GQKGGG

SEQ ID NO: 107 - protein, 6 aa, synthetic construct
  REGION 1..6: note = synthetic amino acid sequence
SGSGGS

SEQ ID NO: 108 - protein, 6 aa, synthetic construct
  REGION 1..6: note = synthetic amino acid sequence
LHDGSD

SEQ ID NO: 109 - protein, 6 aa, synthetic construct
  REGION 1..6: note = synthetic amino acid sequence
GGDGYN

SEQ ID NO: 110 - protein, 6 aa, synthetic construct
  REGION 1..6: note = synthetic amino acid sequence
ASSGDD

SEQ ID NO: 111 - protein, 5 aa, synthetic construct
  REGION 1..5: note = synthetic amino acid sequence
RHAGQ

SEQ ID NO: 112 - protein, 6 aa, synthetic construct
  REGION 1..6: note = synthetic amino acid sequence
GASGNA

SEQ ID NO: 113 - protein, 6 aa, synthetic construct
  REGION 1..6: note = synthetic amino acid sequence
YSDGGA

SEQ ID NO: 114 - protein, 6 aa, synthetic construct
  REGION 1..6: note = synthetic amino acid sequence
AGNGSY

SEQ ID NO: 115 - protein, 6 aa, synthetic construct
  REGION 1..6: note = synthetic amino acid sequence
SSNGSG

SEQ ID NO: 116 - protein, 6 aa, synthetic construct
  REGION 1..6: note = synthetic amino acid sequence
ASAGCT

SEQ ID NO: 117 - protein, 6 aa, synthetic construct
  REGION 1..6: note = synthetic amino acid sequence
SAAGDY

SEQ ID NO: 118 - protein, 6 aa, synthetic construct
  REGION 1..6: note = synthetic amino acid sequence
TAAGSD

SEQ ID NO: 119 - protein, 6 aa, synthetic construct
  REGION 1..6: note = synthetic amino acid sequence
SGNGGA

(19) United States
(12) Patent Application Publication
(10) Pub. No.: US 2026/0276657 A1
(43) Pub. Date: Sep. 17, 2026

(54) TARGETS AND METHODS OF DIAGNOSING, MONITORING AND TREATING FRONTOTEMPORAL DEMENTIA

(71) Applicants: Michael Sierks, Ft. Mcdowell, AZ (US); Stephanie Williams, Phoenix, AZ (US)

(72) Inventors: Michael Sierks, Ft. Mcdowell, AZ (US); Stephanie Williams, Phoenix, AZ (US)

(73) Assignee: ARIZONA BOARD OF REGENTS ON BEHALF OF ARIZONA STATE UNIVERSITY, Scottsdale, AZ (US)

(21) Appl. No.: 19/697,497

(22) Filed: Jun. 3, 2026

Related U.S. Application Data
(62) Division of application No. 17/618,869, filed on Jun. 12, 2020.
(60) Provisional application No. 62/861,003, filed on Jun. 13, 2019.

(51) Int. Cl. G01N 33/68; G01N 33/53

(52) U.S. Cl. CPC G01N 33/6896; G01N 33/53; G01N 2333/4709; G01N 2800/30

(57) ABSTRACT

Disclosed herein are antibodies, antibody fragments, binding agents, and compositions that specifically recognize protein variant biomarkers associated with frontotemporal dementia, kits and methods of use, including diagnosing, monitoring and treating frontotemporal dementia.

### CROSS REFERENCE TO RELATED APPLICATION

This application is a divisional application of U.S. patent application Ser. No. 17/618,869, filed on Dec. 13, 2021, which is the U.S. National Stage of International Patent Application No. PCT/US2020/037603, filed on Jun. 12, 2020, which claims priority to U.S. Provisional Patent Application No. 62/861,003, filed on Jun. 13, 2019, the contents of each of which are hereby incorporated by reference in their entireties.

### ACKNOWLEDGMENT OF GOVERNMENT SUPPORT

This invention was made with government support under AG042066 and AG054048 awarded by the National Institutes of Health. The government has certain rights in the invention.

### FIELD

This disclosure relates to antibodies, antibody fragments, single-chain variable fragments, binding agents, and compositions that specifically recognize protein variant biomarkers associated with frontotemporal dementia (FTD), and methods of use, including diagnosing, monitoring and treating frontotemporal dementia.

### INCORPORATION-BY-REFERENCE OF MATERIAL ELECTRONICALLY FILED

The official copy of the sequence listing is submitted electronically in an xml file format having the file name “11157-100SeqList.xml” created on May 20, 2026, and having a size of 151,041 bytes, and is filed concurrently with the specification. The sequence listing is part of the specification and is herein incorporated by reference in its entirety.

### BACKGROUND

Early protein misfolding and aggregation is behind many neurodegenerative diseases, including AD, Parkinson's disease (PD), Frontal Temporal Dementia (FTD), Lewy Body Dementia (LBD), and Huntington's disease (HD) among others. While each disease has been primarily associated with aggregation of a specific protein; beta-amyloid with AD, alpha-synuclein (a-syn) with PD and LBD, tau with various tauopathies including AD and FTD, TDP-43 with amyotrophic lateral sclerosis (ALS) and FTD, and huntingtin with HD, more than one protein is likely to misfold and aggregate in brain tissue complicating diagnosis and treatment strategies. While all these proteins can form fibrillar aggregates, they can also form a variety of different smaller soluble aggregate structures as well, and increasing evidence implicates small soluble oligomeric forms of these different proteins as the relevant toxic species in the various diseases rather than the fibrillar aggregates that serve as diagnostic hallmarks. Since cellular stress induced by misfolding and aggregation of one protein such as beta-amyloid may well lead to misfolding and aggregation of other proteins such as tau and a-syn, the presence of multiple misfolded proteins in different diseases should be expected. Therefore characterizing which aggregated protein species are present at different stages of each disease would greatly facilitate identification of suitable biomarkers and development of better diagnostic and treatment strategies for these neurodegenerative diseases.

Accordingly, there exists the need for new therapies and reagents for the diagnosis and treatment of neurodegenerative diseases, including frontotemporal dementia (FTD).

### SUMMARY

Reagents that can selectively recognize protein variant biomarkers in frontotemporal dementia (FTD) are fundamental for designing effective therapeutic strategies to prevent or impede disease progression. Here, the inventors analyzed sera samples from 12 FTD cases with TAR DNA-binding protein 43 (TDP-43) proteinopathy and 12 FTD cases with tau proteinopathy using a panel of 14 scFvs. Seven of the scFvs bound select protein variants of TDP-43, three bound select oligomeric variants of beta-amyloid, two bound select oligomeric variants of tau and two bound select oligomeric variants of alpha-synuclein. Not every scFv recognized every case but all 24 FTD-TDP and FTD-Tau cases were selected with the panel. Some of the scFvs were significantly more reactive with the FTD-Tau cases compared to FTD-TDP cases including the TDP-43 reactive scFvs AD-TDP2 and AD-TDP3 and the oligomeric tau reactive scFv F9T. The FTD-TDP1 and FTD-TDP2 reactive scFvs were significantly more reactive with both the FTD-TDP and FTD-Tau cases compared to the controls. Most surprising, the oligomeric beta-amyloid reactive scFvs A4 and E1 displayed strong sensitivity and specificity for all 24 cases alluding to a possible role of beta-amyloid oligomers in FTD pathology. FTD-TDP cases generated excellent negative correlations between MMSE scores and select protein variants while FTD-Tau cases produced excellent negative correlations between UPDRS motor scores and select protein variants. Similarly, excellent negative correlations were detected between select protein variants levels and tau tangle levels in the FTD-Tau cases. APOE genotype did not seem to influence protein variants levels, in particular beta-amyloid oligomer levels like in Alzheimer's disease (AD). No major differences were detected between males and females in the FTD-TDP cases and only slight differences in the FTD-Tau cases. Overall, diverse binding patterns between FTD-TDP and FTD-Tau cases were observed as well as across the cases present within each subgroup. These studies indicate that FTD cases can benefit from personalized diagnostic tests and therapeutics strategies. Moreover, by utilizing the disclosed panel system one is creating a more personalize approach for FTD patients centered on a wider range of targets to create the most effective treatment plan. The panel indicates which FTD patients are being affected by oligomeric beta-amyloid pathology.

Based on the results disclosed herein, the presented scFvs can serve as excellent indicators of progressing pathology and may function therapeutically to further prevent disease advancement. As such, disclosed herein are antibodies, antibody fragments, binding agents and compositions that specifically recognize protein variant biomarkers associated with frontotemporal dementia (FTD), kits and methods of use, including diagnosing, monitoring and treating frontotemporal dementia.

The foregoing and other features and advantages of the disclosure will become more apparent from the following detailed description, which proceeds with reference to the accompanying figures.

### BRIEF DESCRIPTION OF DRAWINGS

FIGS. 1A-1N provide a series of bar graphs illustrating the results of the ELISA analyses.

FIG. 10 provides a bar graph illustrating cumulative protein variants in control and sera samples.

FIGS. 2A-2F are a series of plots illustrating Reactive Variants and Oligomers relative to MMSE Score.

FIGS. 2G and 2H a set of bar graphs illustrating the levels of the different protein variants and pathological findings including Braak stage.

FIGS. 3A-3I provide a series of plots illustrating one-tailed bivariate correlations between MMSE scores and protein variants content.

FIGS. 3J and 3K provide bar graphs illustrating a set of bar graphs illustrating the levels of the different protein variants and pathological findings including plaque levels (3J) and tangle levels (3K).

FIGS. 4A-4D a series of plots illustrating one-tailed bivariate correlations between amount of reactive oligomers or variant and brain weight.

FIGS. 5A-5J a series of plots illustrating one-tailed bivariate correlations between amount of reactive oligomers or variant and tangles.

FIGS. 6A-6D a series of bar graphs illustrating the correlation between cumulative protein variants and APOE Genotype (6A and 6B) and Gender (6C and 6D).

FIG. 7 provides Table 1, Demographics and Medical History.

FIG. 8 provides Table 2, Diagnostic Proficiency of ScFvs.

FIGS. 9A-9C are Atomic Force Microscopy (AFM) panning images. FIG. 9A shows phage binding to BSA prior to subtractive panning to get rid of non-specific binders, FIG. 9B no phage binding is observed after multiple rounds of subtractive panning with healthy control tissue, and FIG. 9C phage binding with FTD-TDP IP after positive selection.

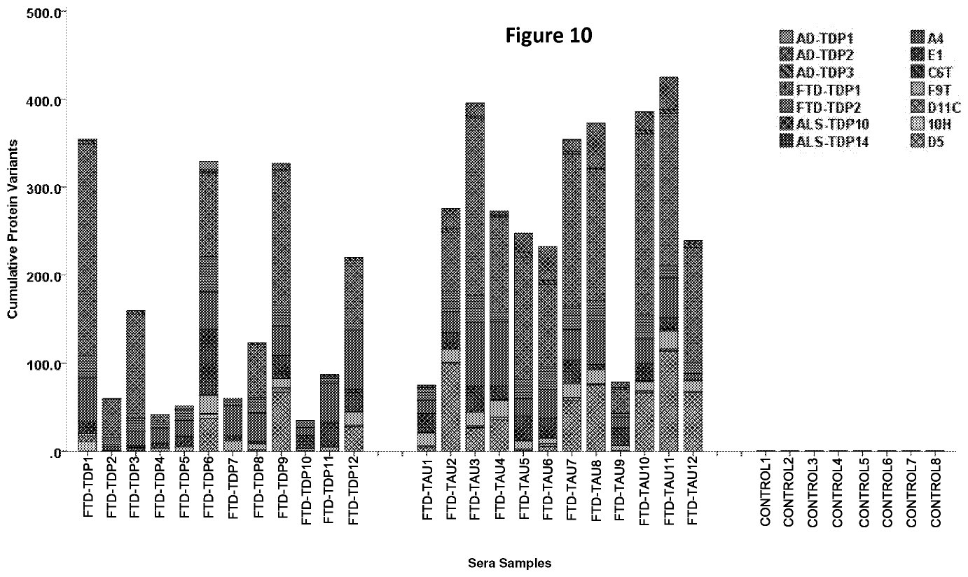

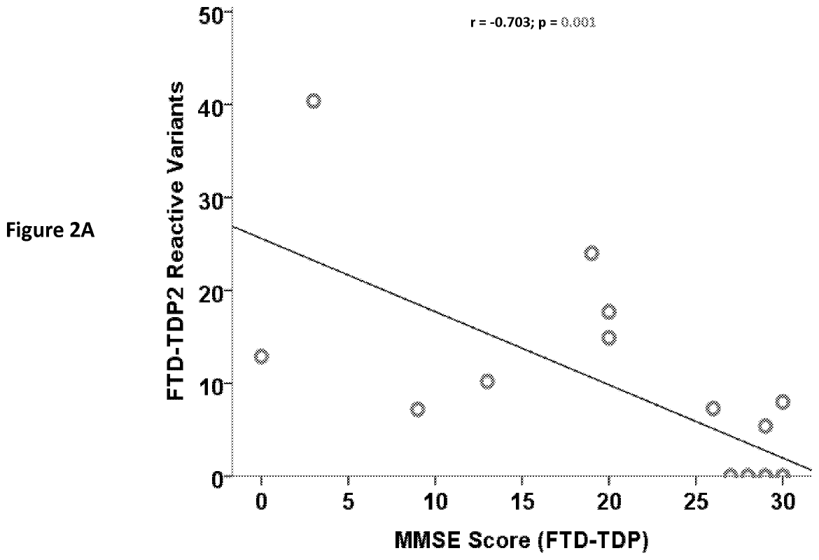

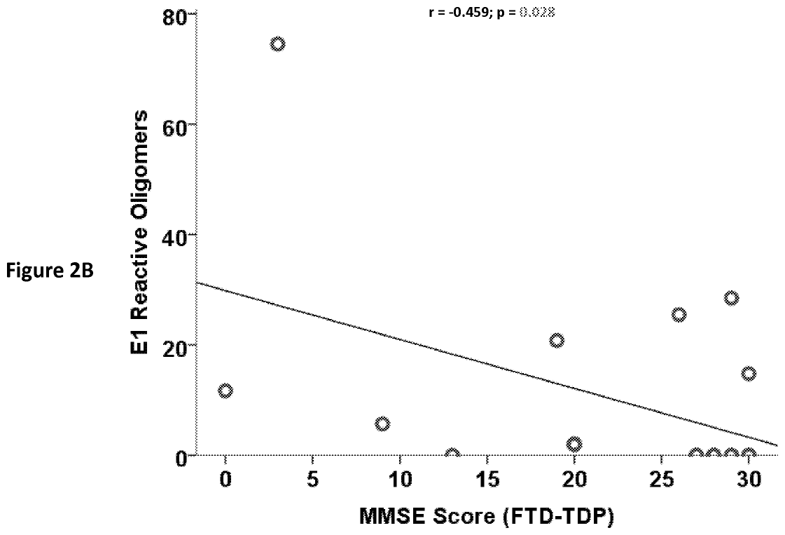

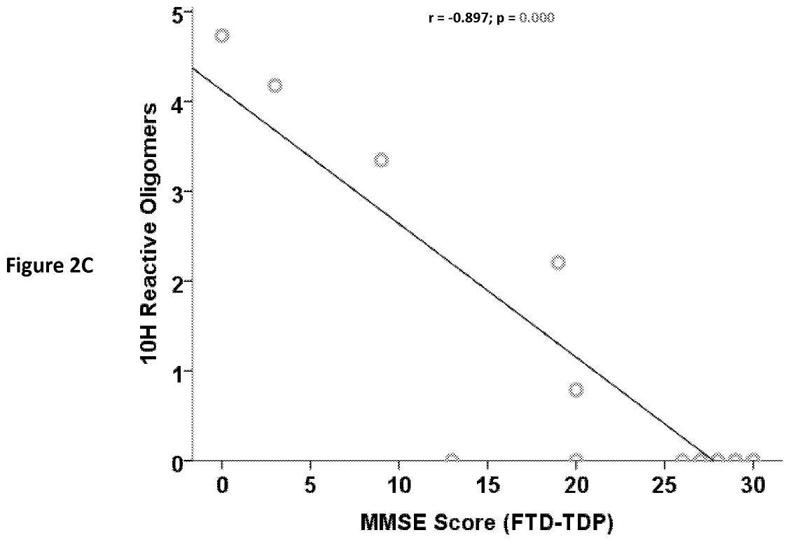

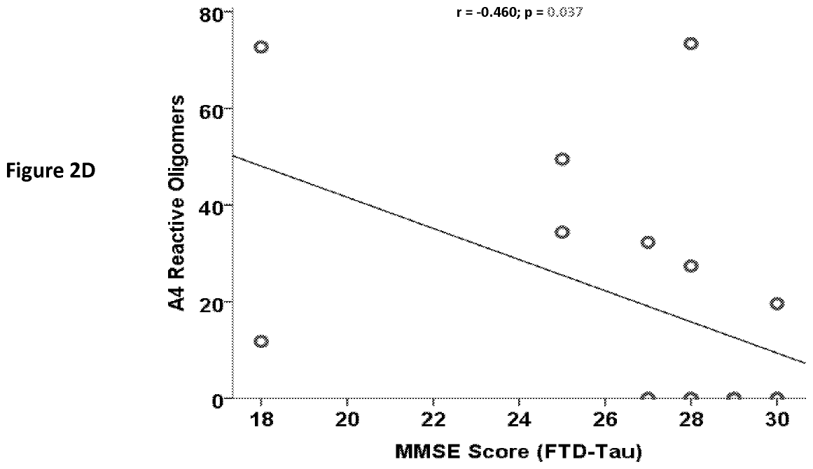

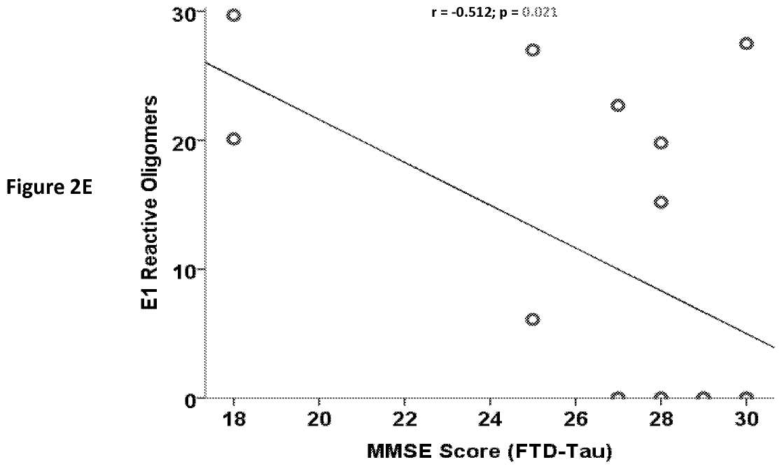

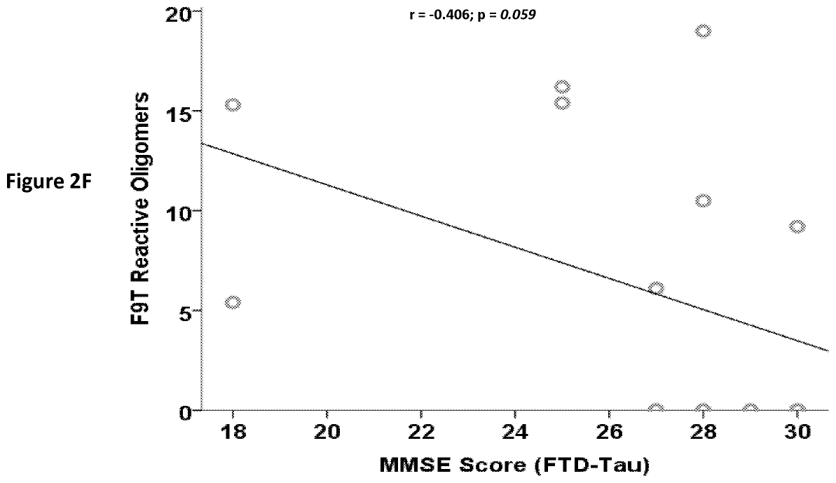

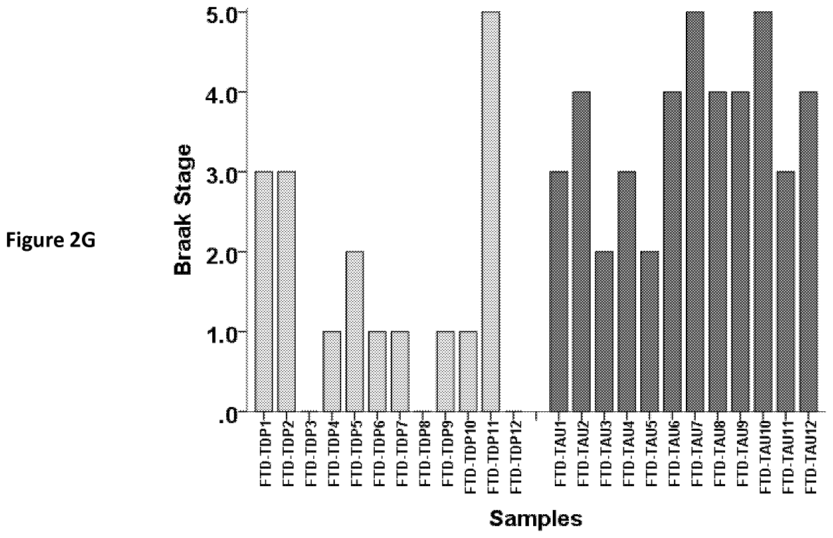

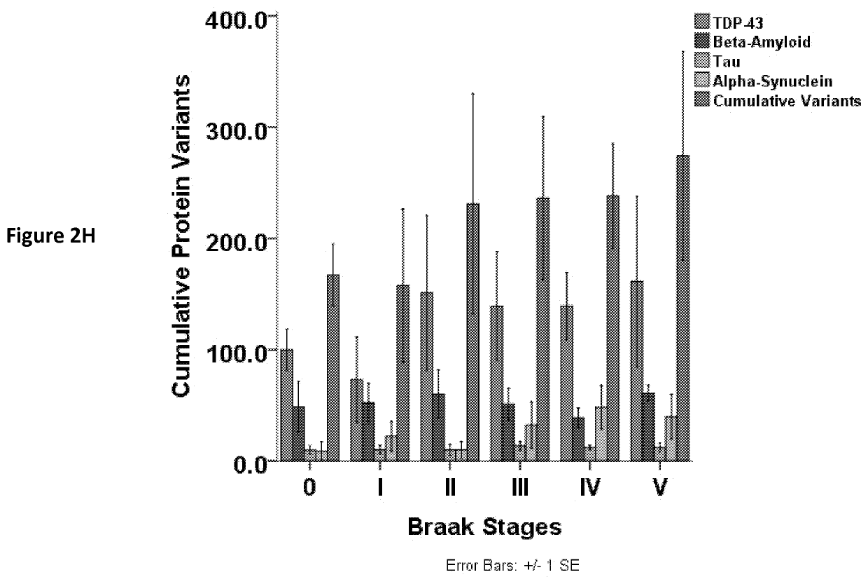

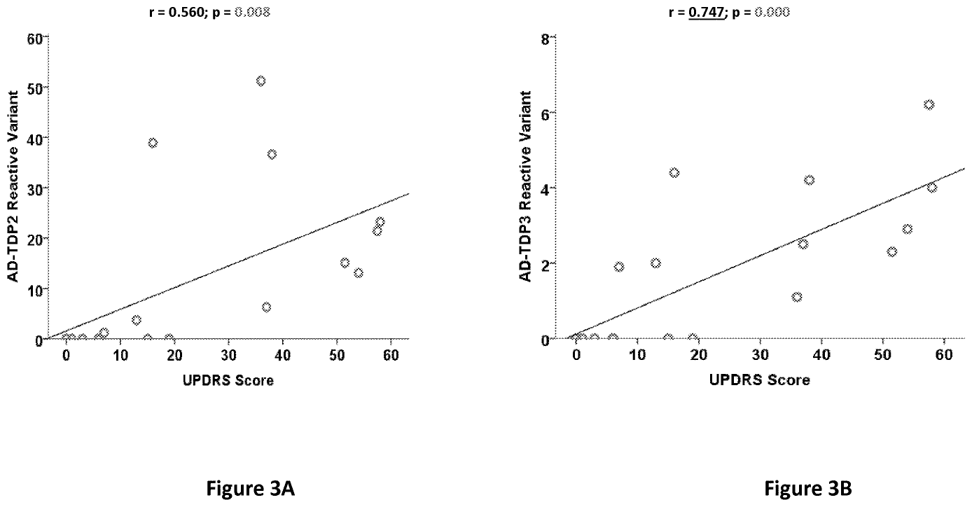

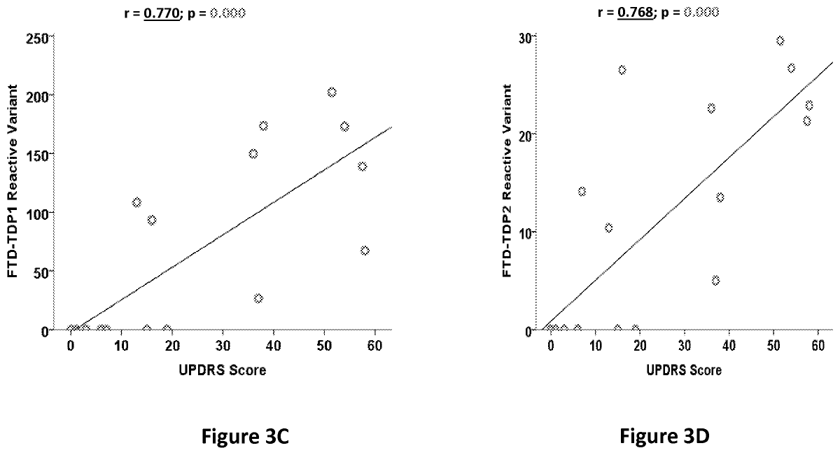

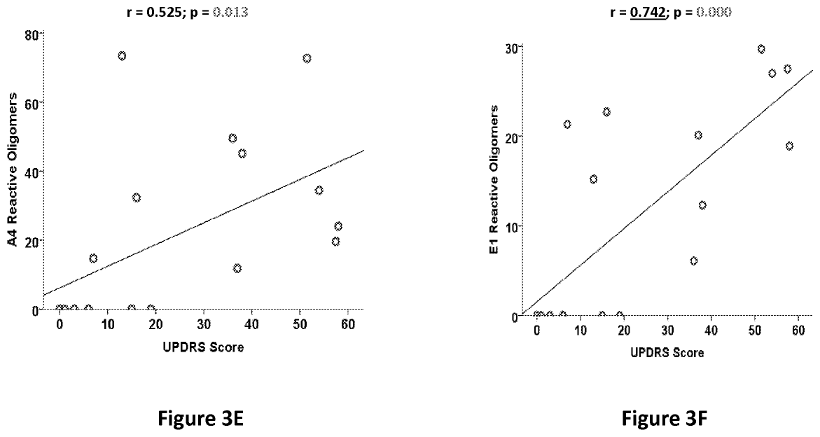

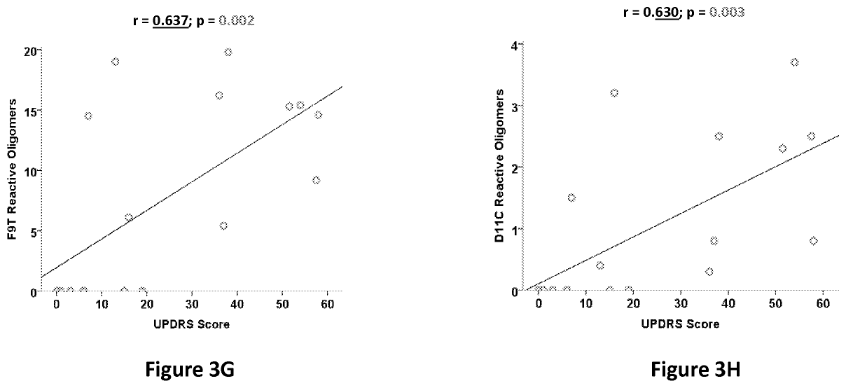

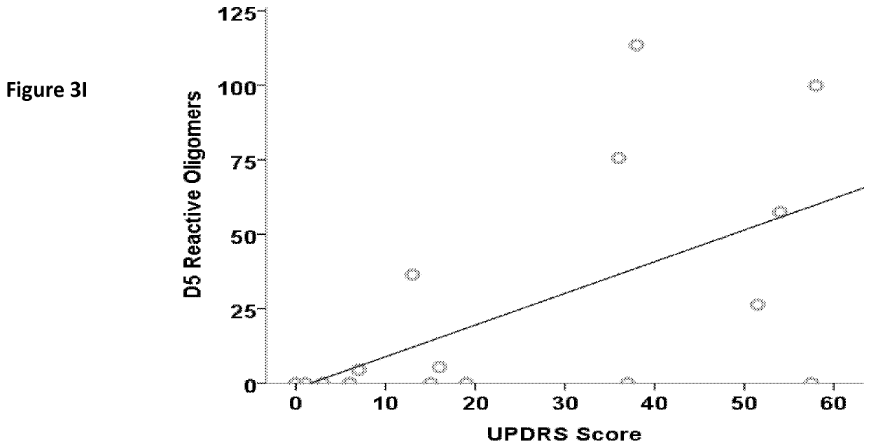

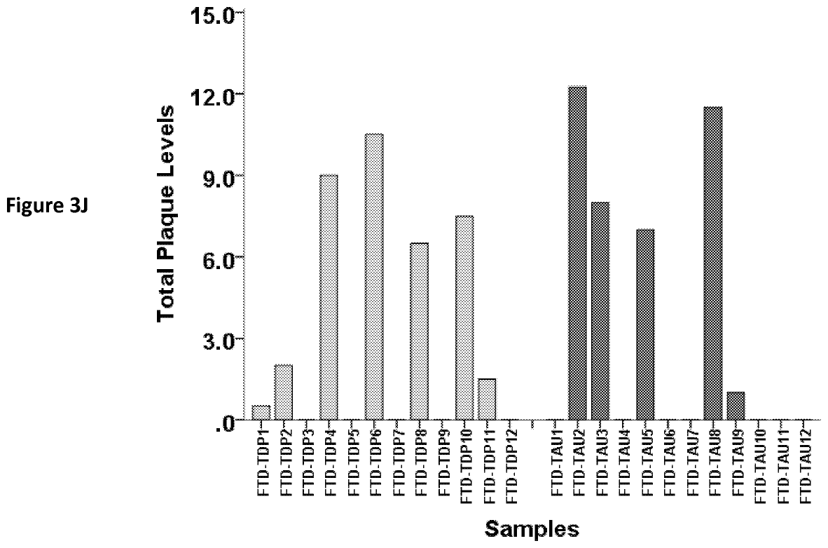

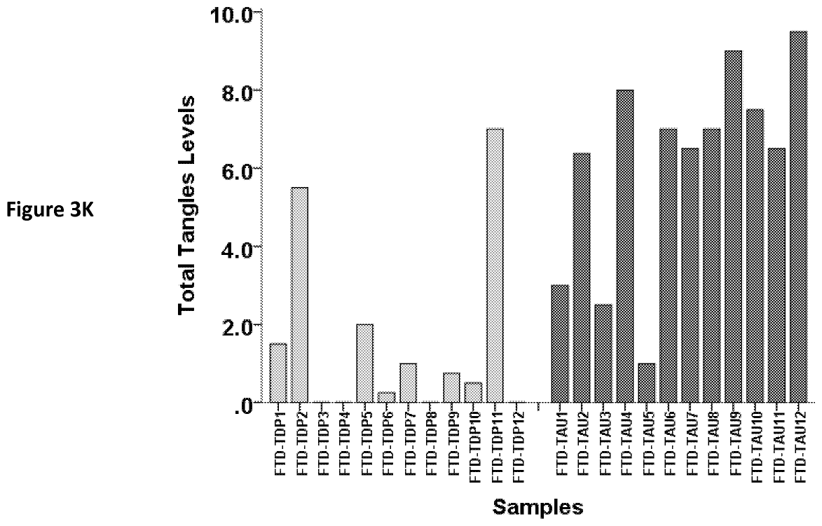

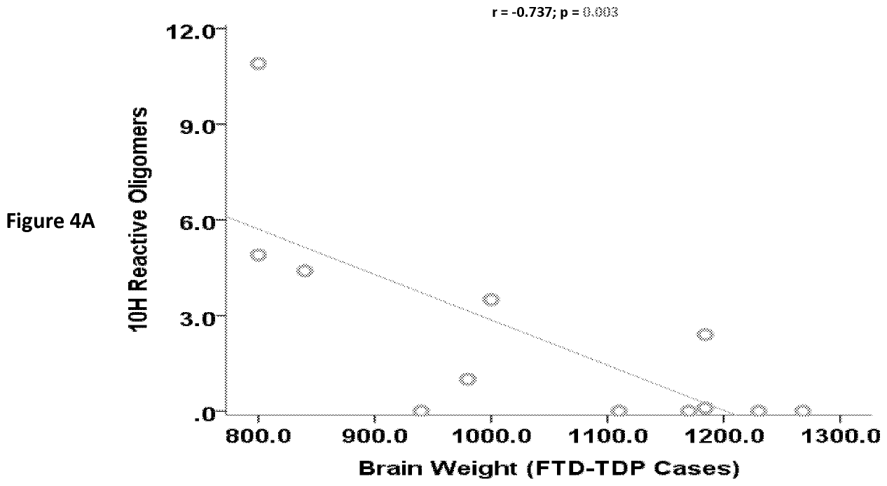

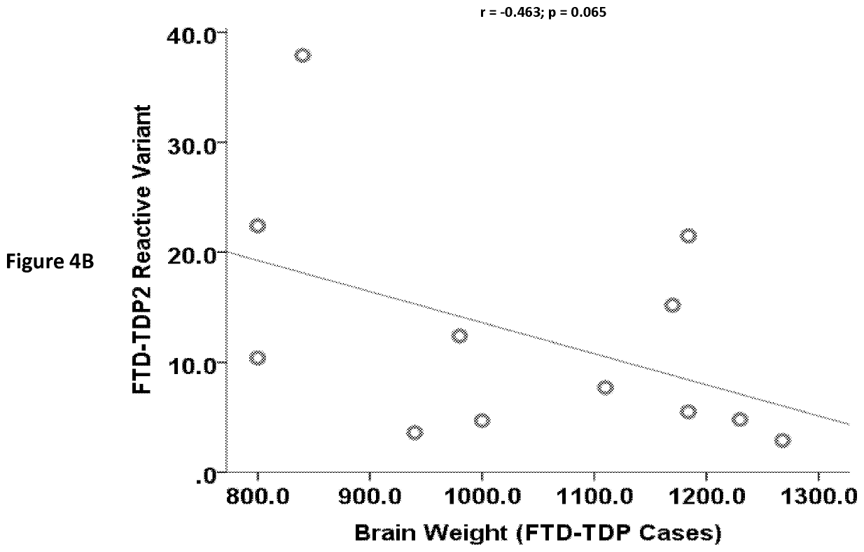

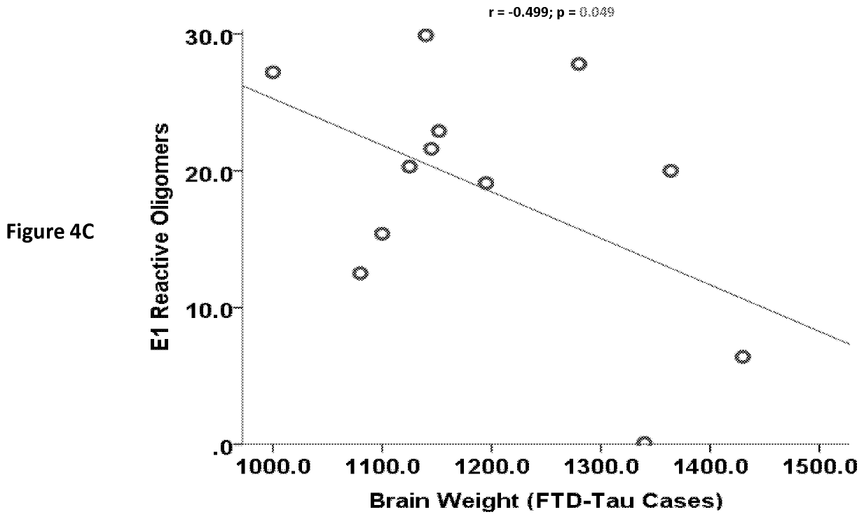

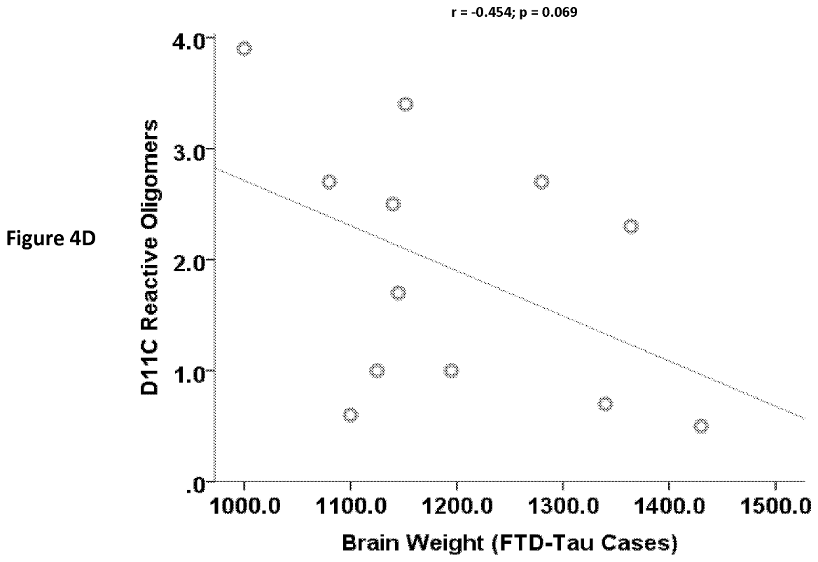

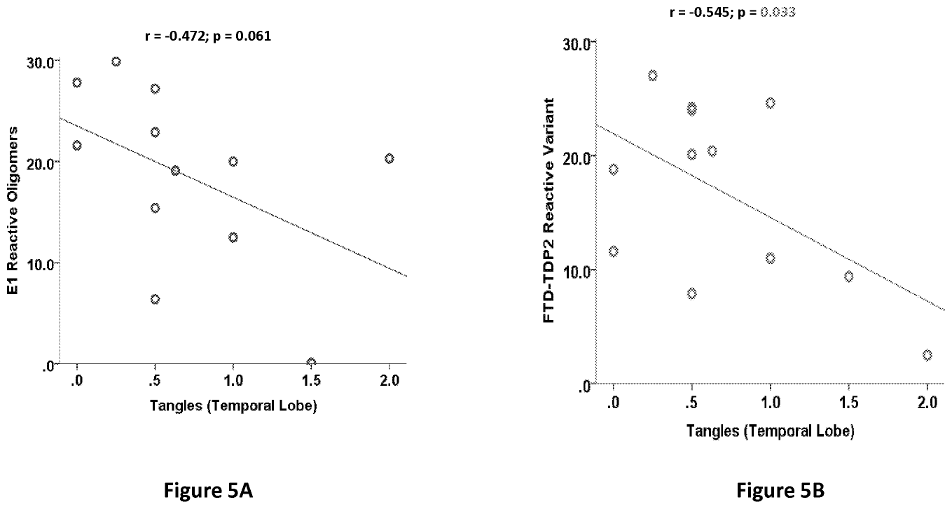

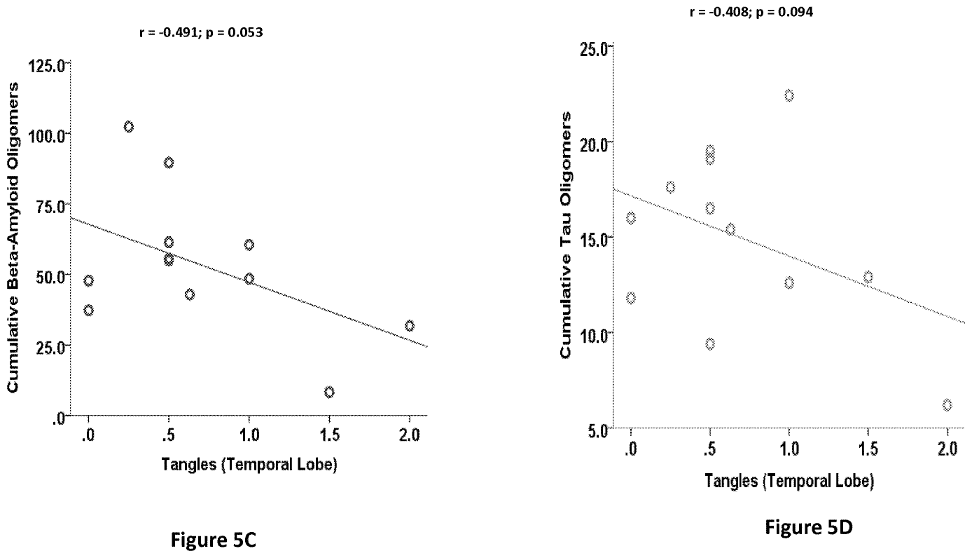

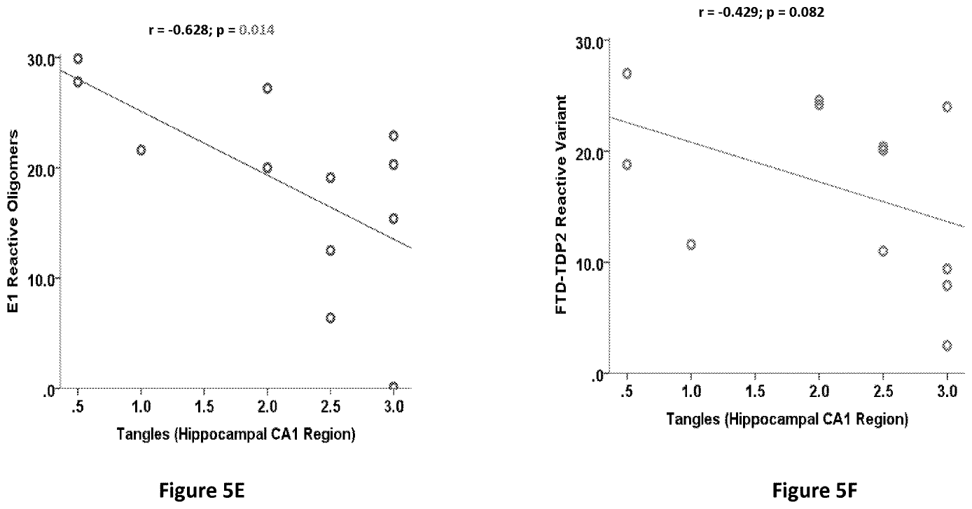

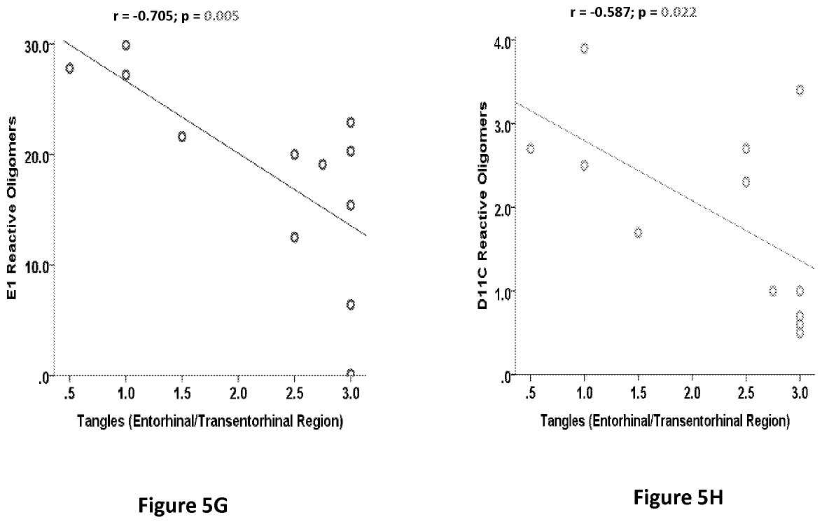

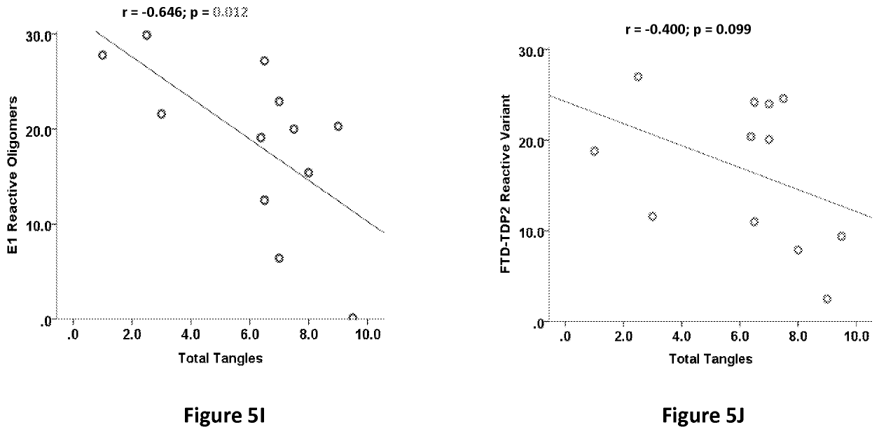

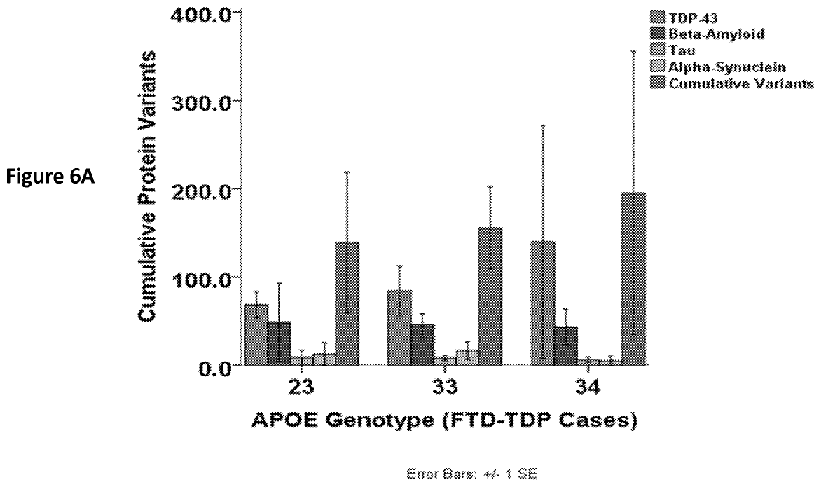

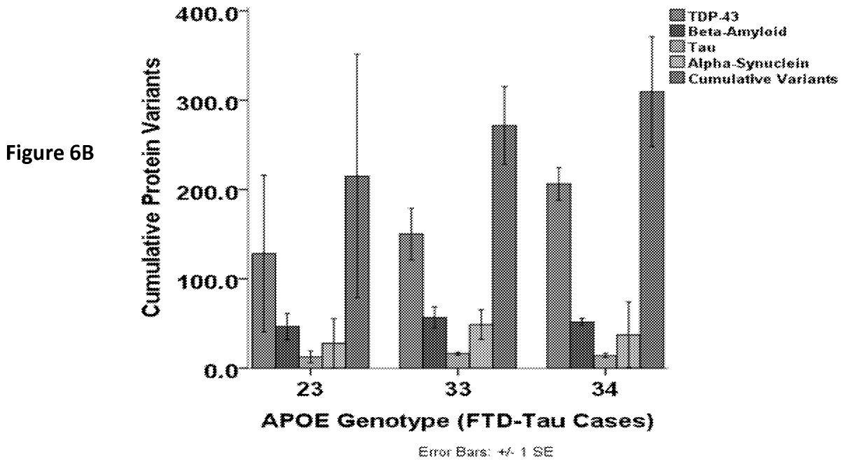

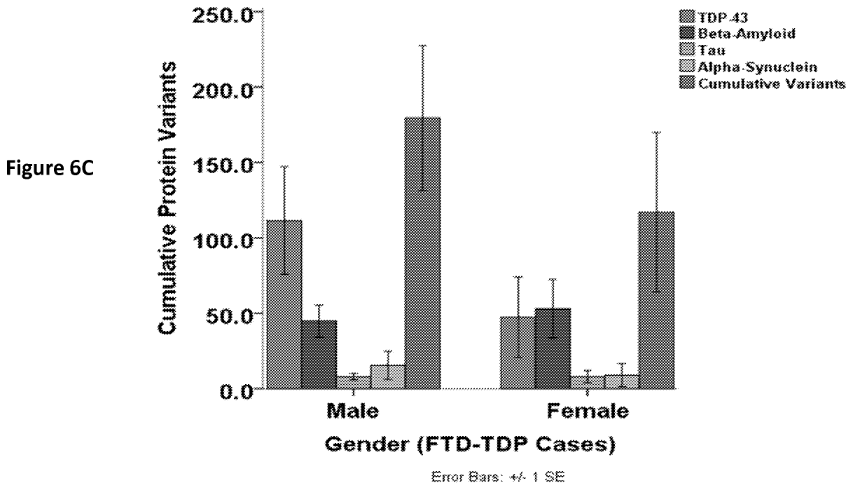

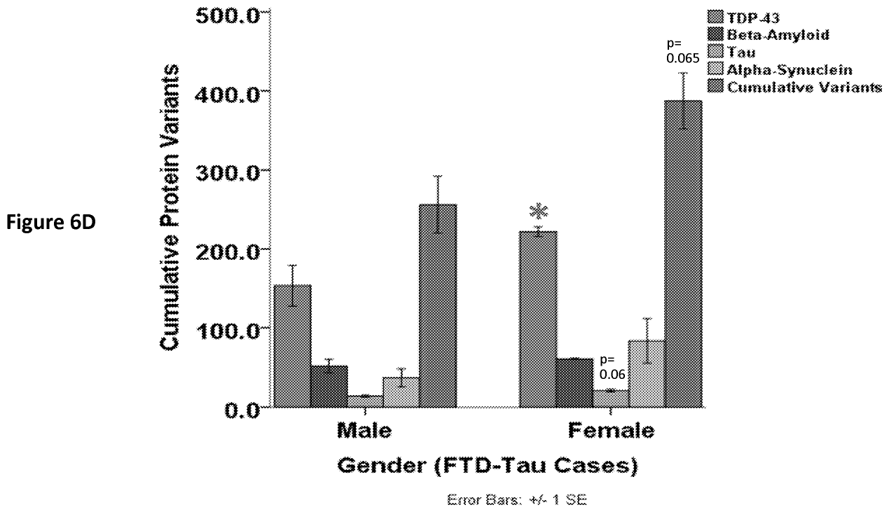

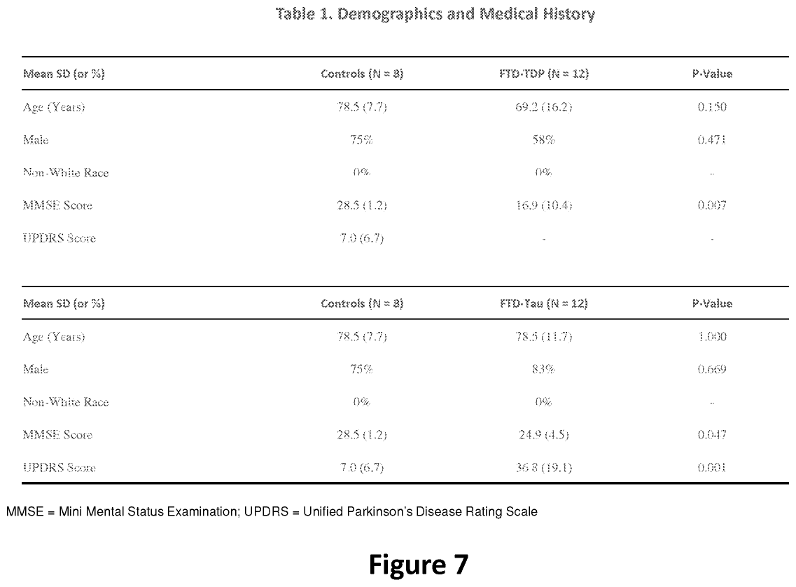

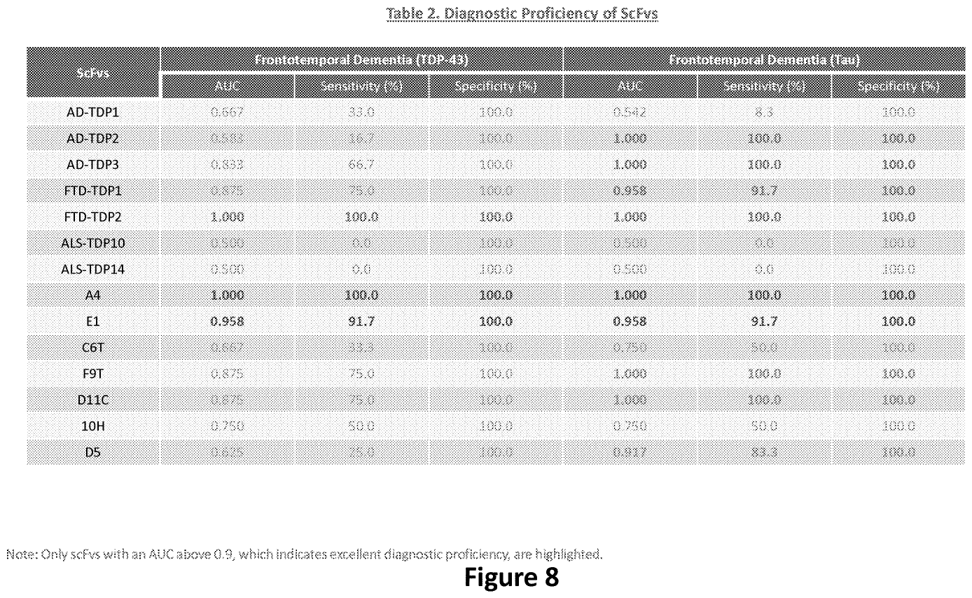

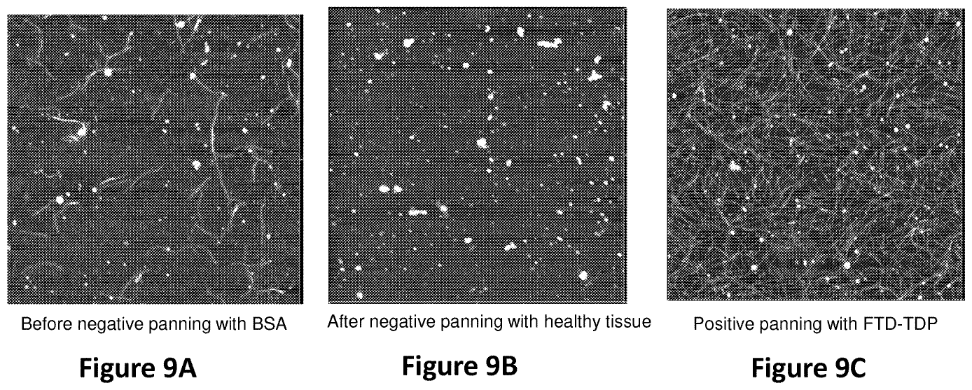

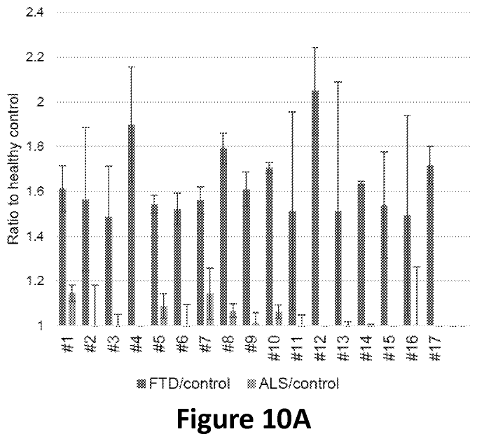

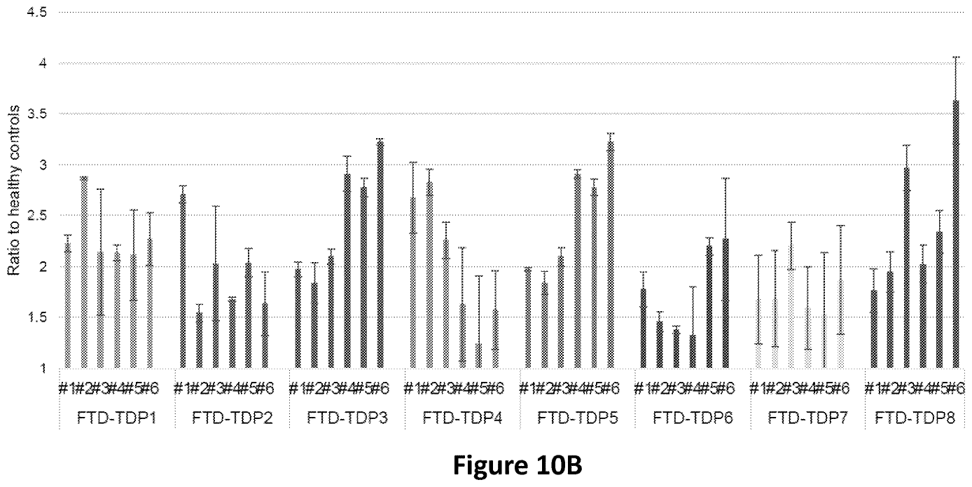

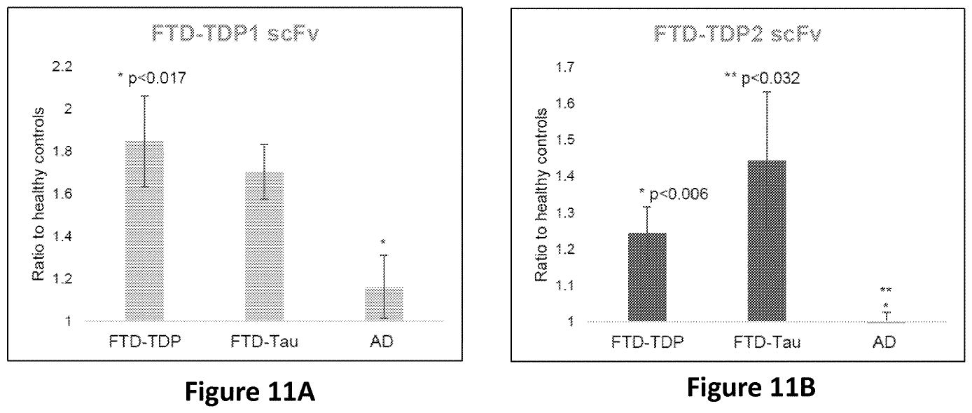

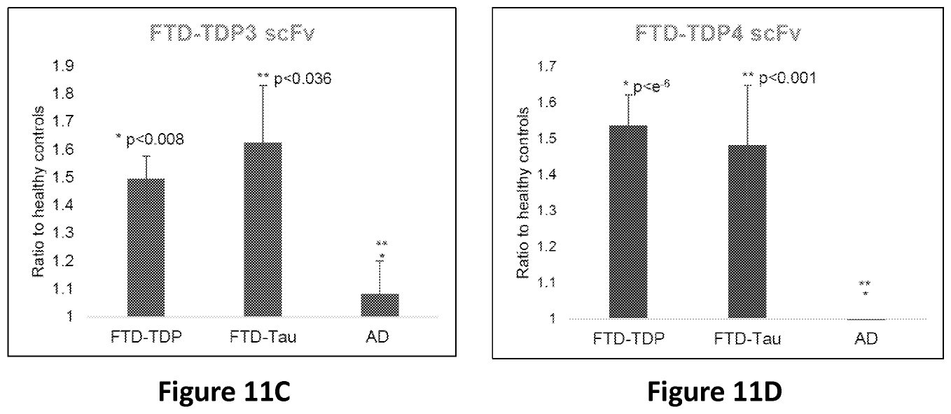

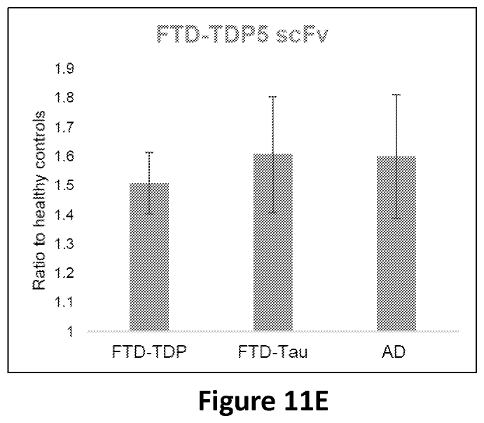

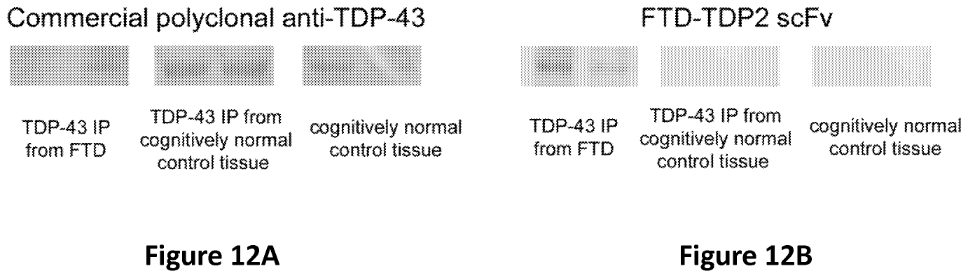

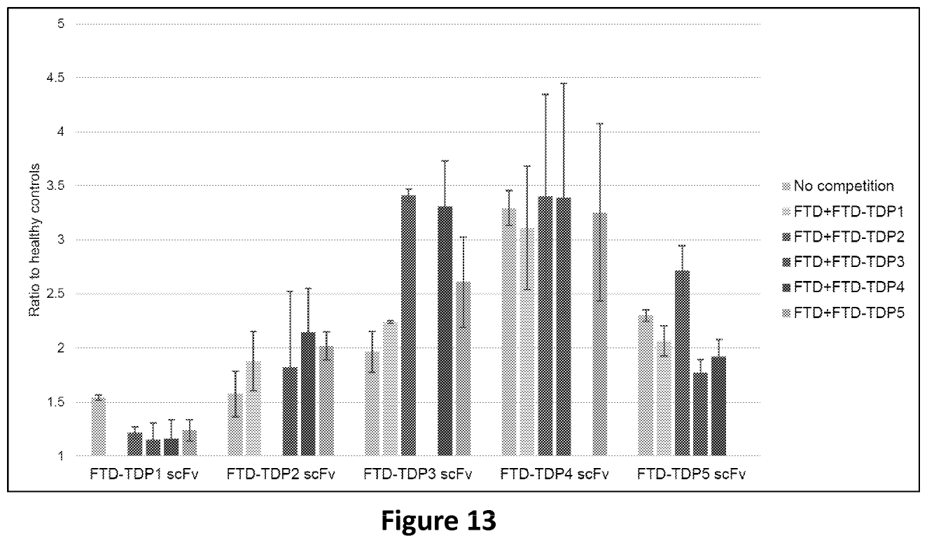

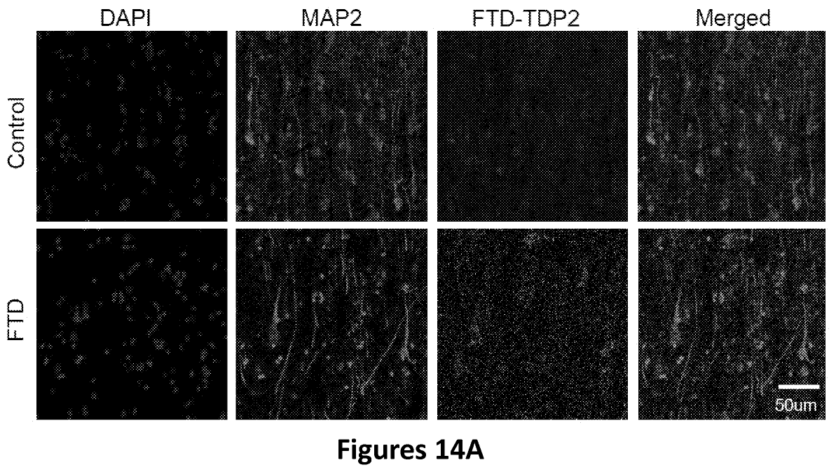

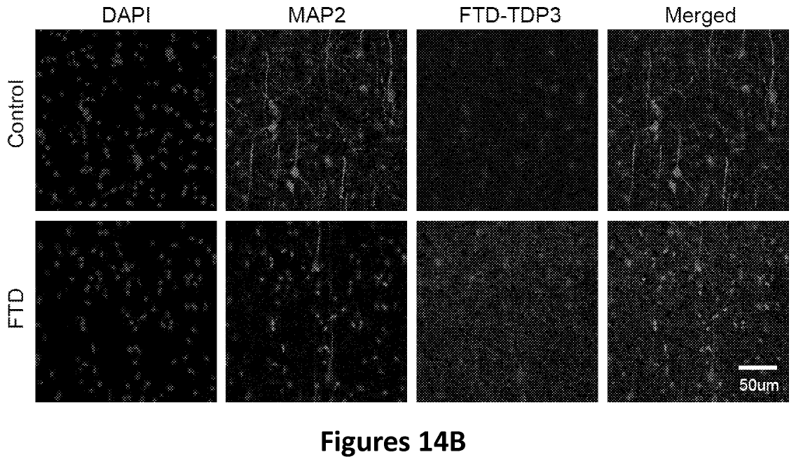

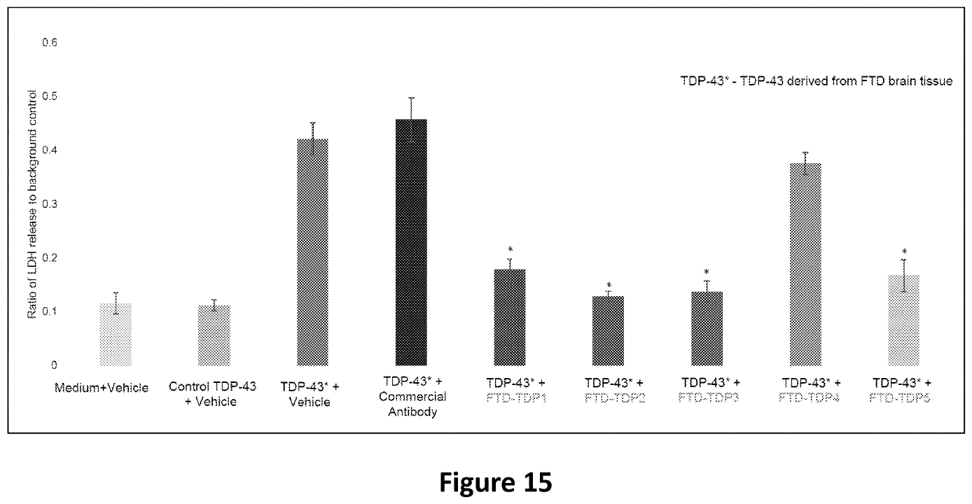

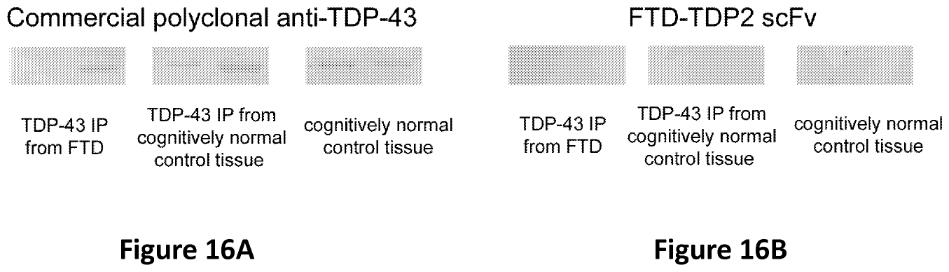

FIGS. 10A and 10B show screening of anti-TDP phage with pooled and individual FTD and control brain tissue homogenates. Phage obtained after positive selection against human FTD brain derived TDP variants were screened with: FIG. 10A, pooled human FTD brain tissue samples (n=3), pooled ALS brain tissue samples (n=3) and healthy controls (n=2);

FIG. 10B, selected 8 phages were further screened with individual FTD brain tissue homogenates (n=6) and healthy controls.

FIGS. 11A-11E show graphs of Anti-TDP scFvs characterization with FTD and control sera. Reactivity of anti-TDP scFvs with FTD and AD sera were assessed using sandwich ELISA. 4 of the 5 anti-TDP scFvs selectively bind to both FTD-TDP (n=12) and FTD-tau (n=12) sera and have relatively little to no binding to AD sera (n=11). FTDP-TDP5 was the only scFv that had reactivity with FTD-TDP, FTD-Tau and AD sera over cognitively normal healthy controls.

FIGS. 12A and 12B show Western Blot Analysis results. Reactivity against healthy control tissue and TDP-43 immunoprecipitated from healthy controls and FTD was assessed under non-reducing and non-denaturing conditions with: FIG. 12A) Commercial TDP antibody identifying TDP variants in FTD and healthy controls; and FIG. 12B) FTD-TDP2 scFv which recognizes disease variant of TDP (~70 kDa) present in FTD and not healthy controls.

FIG. 13 shows a competition ELISA of anti-TDP scFvs. X-axis represents each scFv and Y-Axis represents ratio to age matched controls. Each scFv was tested with FTD sera (1 FTD-TDP+1 FTD-tau) (no competition) or FTD sera pre-incubated with each of the other four scFvs (competition). One-way ANOVA analysis indicate no significant difference between the no competition and competing scFvs.

FIGS. 14A and 14B show immunohistochemistry with anti-TDP scFvs. Tissue sections were incubated with FTD-TDP2 and FTD-TDP3 respectively (1:100) on a shaking stage overnight at 4° C. Primary antibodies against c-myc region of scFv (Sigma, 1:1000, rabbit) and MAP2 (Covance, 1:400, mouse) were applied followed by goat anti-rabbit IgG (green) and goat anti-mouse IgG (red) with fluorescence. The sections were observed and imaged with Leica SP5.

FIG. 15 illustrate therapeutic potential of anti-TDP scFvs. SH-SY5Y neuroblastoma cell line was treated with TDP-IP derived from human FTD and control brain tissue. The cells were further treated with a commercial anti-TDP antibody (ab 190963, Abcam, 1 μg/mL) or anti TDP scFvs (FTD-TDP1, FTD-TDP2, FTD-TDP4 and FTD-TDP5) for 12 hours. The cell damage and toxicity were tested by measuring lactate dehydrogenase (LDH). Neither the commercial TDP antibody or FTD-TDP4 significantly blocked toxicity of the FTD brain derived TDP IP at the concentration studied, while FTD-TDP1, FTD-TDP2, FTD-TDP3 and FTD-TDP5 all significantly inhibited the TDP induced toxicity.

FIGS. 16A and 16B show Western blot under denaturing conditions. Reactivity against healthy control tissue and TDP-43 immunoprecipitated from healthy controls and FTD was assessed under reducing and denaturing conditions with FIG. 16A) Commercial TDP antibody, and FIG. 16B) FTD-TDP2 scFv. While commercial antibody recognizes TDP variants in FTD and healthy controls, FTD-TDP2 scFv does not recognize TDP variants in any of the samples.

### DETAILED DESCRIPTION OF SEVERAL EMBODIMENTS

This technology disclosed herein is described in one or more exemplary embodiments in the following description with reference to the Figures. Reference throughout this specification to “one embodiment,” “an embodiment,” or similar language means that a particular feature, structure, or characteristic described in connection with the embodiment is included in at least one embodiment of the present technology disclosed herein. Thus, appearances of the phrases “in one embodiment,” “in an embodiment,” and similar language throughout this specification may, but do not necessarily, all refer to the same embodiment.

The described features, structures, or characteristics of the technology disclosed herein may be combined in any suitable manner in one or more embodiments. In the following description, numerous specific details are recited to provide a thorough understanding of embodiments of the technology disclosed herein. One skilled in the relevant art will recognize, however, that the technology disclosed herein may be practiced without one or more of the specific details, or with other methods, components, materials, and so forth. In other instances, well-known structures, materials, or operations are not shown or described in detail to avoid obscuring aspects of the technology disclosed herein.

The following explanations of terms and methods are provided to better describe the present compounds, compositions and methods, and to guide those of ordinary skill in the art in the practice of the present disclosure. It is also to be understood that the terminology used in the disclosure is for the purpose of describing particular embodiments and examples only and is not intended to be limiting.

As used herein, the singular forms “a,” “an,” and “the” are intended to include the plural forms as well, unless the context clearly indicates otherwise.

As used herein, the term “and/or” refers to and encompasses any and all possible combinations of one or more of the associated listed items, as well as the lack of combinations when interpreted in the alternative (“or”).

As used herein, “one or more” or at least one can mean one, two, three, four, five, six, seven, eight, nine, ten or more, up to any number.

As used herein, the term “comprises” means “includes.” Hence “comprising A or B” means including A, B, or A and B. It is further to be understood that all base sizes and all molecular weight or molecular mass values given for peptides and nucleic acids are approximate and are provided for description.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

Unless otherwise noted, technical terms are used according to conventional usage. Definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710); and other similar references.

Suitable methods and materials for the practice or testing of this disclosure are described below. Such methods and materials are illustrative only and are not intended to be limiting. Although methods and materials similar or equivalent to those described herein can be used in the practice or testing of the present disclosure, suitable methods and materials are described below. All publications, patent applications, patents, and other references mentioned herein are incorporated by reference in their entirety. Unless otherwise defined, all technical terms used herein have the same meaning as commonly understood. Other methods and materials similar or equivalent to those described herein can be used. For example, conventional methods well known in the art to which this disclosure pertains are described in various general and more specific references, including, for example, Sambrook et al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999. In addition, the materials, methods, and examples are illustrative only and not intended to be limiting.

In order to facilitate review of the various embodiments of this disclosure, the following explanations of specific terms are provided:

Alteration or difference: An increase or decrease in the amount of something, such as a protein antigen. In some examples, the difference is relative to a control or reference value or range of values, such as an amount of a protein that is expected in a subject who does not have a particular condition or disease being evaluated. Detecting an alteration or differential expression/activity can include measuring a change in protein expression, concentration or activity, such as by ELISA, Western blot and/or mass spectrometry.

Animal: Living multi-cellular vertebrate organisms, a category that includes, for example, mammals and birds. The term mammal includes both human and non-human mammals. Similarly, the term “subject” includes both human and veterinary subjects, for example, mice.

Antibody: An immunoglobulin, antigen-binding fragment, or derivative thereof, that specifically binds and recognizes an analyte (antigen) such as influenza HA. The term “antibody” is used herein in the broadest sense and encompasses various antibody structures, including but not limited to monoclonal antibodies, polyclonal antibodies, multispecific antibodies (e.g., bispecific antibodies), and antibody fragments, so long as they exhibit the desired antigen-binding activity.

Non-limiting examples of antibodies include, for example, intact immunoglobulins and variants and fragments thereof known in the art that retain binding affinity for the antigen. Examples of antibody fragments include but are not limited to Fv, Fab, Fab′, Fab′-SH, F(ab′)2; diabodies; linear antibodies; single-chain antibody molecules, such as single-chain variable fragments (e.g., scFvs); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2^{nd} Ed., Springer Press, 2010).

A single-chain variable fragment (scFv) is a genetically engineered molecule containing the VH and VL domains of one or more antibody(ies) linked by a suitable polypeptide linker as a genetically fused single chain molecule (see, for example, Bird et al, Science, 242:423-426, 1988; Huston et al, Proc. Natl. Acad. Sci., 85:5879-5883, 1988; Ahmad et al, Clin. Dev. Immunol, 2012, doi: 10.1 155/2012/980250; Marbry, IDrugs, 13:543-549, 2010). The intramolecular orientation of the VH-domain and the VL-domain in a scFv, is typically not decisive for scFvs. Thus, scFvs with both possible arrangements (VH-domain-linker domain-VL-domain; VL-domain-linker domain-VH-domain) may be used.

In a dsFv the VH and VL have been mutated to introduce a disulfide bond to stabilize the association of the chains. Diabodies also are included, which are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et ai, Proc. Natl. Acad. ScL, 90:6444-6448, 1993; Poljak ef ai, Structure, 2:1121-1 123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3^{rd} Ed., W. H. Freeman & Co., New York, 1997.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Antibody competition assays are known, and an exemplary competition assay is provided herein.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain; see, e.g., Kindt et al. Kuby Immunology, 6^{th} ed., W. H. Freeman and Co., page 91 (2007).) In several embodiments, the VH and VL combine to specifically bind the antigen. In additional embodiments, only the VH is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). Any of the disclosed antibodies can include a heterologous constant domain. For example the antibody can include constant domain that is different from a native constant domain, such as a constant domain including one or more modifications (such as the “LS” mutations) to increase half-life.

References to “VH” or “VH” refer to the variable region of an antibody heavy chain, including that of an antigen binding fragment, such as Fv, scFv, dsFv or Fab. References to “VL” or “VL” refer to the variable domain of an antibody light chain, including that of an Fv, scFv, dsFv or Fab.

The VH and VL contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (“Sequences of Proteins of Immunological Interest,” 5th Ed. Public Health Service, National Institutes of Health, Bethesda, MD, 1991; “Kabat” numbering scheme), Al-Lazikani et al, (JMB 273,927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a VH CDR3 is the CDR3 from the VH of the antibody in which it is found, whereas a VLCDR1 is the CDR1 from the VL of the antibody in which it is found. Light chain CDRs are sometimes referred to as LCDR1, LCDR2, and LCDR3. Heavy chain CDRs are sometimes referred to as HCDR1, HCDR2, and HCDR3.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples, monoclonal antibodies are isolated from a subject. Monoclonal antibodies can have conservative amino acid substitutions which have substantially no effect on antigen binding or other immunoglobulin functions. (See, for example, Harlow & Lane, Antibodies, A Laboratory Manual, 2^{nd} ed. Cold Spring Harbor Publications, New York (2013).)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas ef aZ. Phage display: A Laboratory Manuel. 1^{st} Ed. New York: Cold Spring Harbor Laboratory Press, 2004. Print.; Lonberg, Nat. Biotech., 23:1117-1125, 2005; Lonenberg, Curr. Opin. Immunol., 20:450-459, 2008).

A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. An “antigenic polypeptide” is a polypeptide to which an immune response, such as a T cell response or an antibody response, can be stimulated. “Epitope” or “antigenic determinant” refers to a site on an antigen to which B and/or T cells respond. Methods of determining spatial conformation of epitopes include, for example, x-ray crystallography and multi-dimensional nuclear magnetic resonance spectroscopy. The term “antigen” denotes both subunit antigens, (for example, antigens which are separate and discrete from a whole organism with which the antigen is associated in nature), as well as killed, attenuated or inactivated bacteria, viruses, fungi, parasites or other microbes. An “antigen,” when referring to a protein, includes a protein with modifications, such as deletions, additions and substitutions (generally conservative in nature) to the native sequence, so long as the protein maintains the ability to elicit an immunological response, as defined herein. These modifications may be deliberate, as through site-directed mutagenesis, or may be accidental, such as through mutations of hosts which produce the antigens.

Contacting: “Contacting” includes in solution and solid phase. “Contacting” can occur in vitro with, e.g., samples, such as biological samples containing a target biomolecule, such as an antibody. “Contacting” can also occur in vivo.

Diagnosis: The process of identifying a condition or disease by its signs, symptoms, results of various tests and presence of diagnostic indicators. The conclusion reached through that process is also called “a diagnosis.”

Immunoassay: A biochemical test that measures the presence or concentration of a substance in a sample, such as a biological sample, using the reaction of an antibody to its cognate antigen, for example the specific binding of an antibody to a protein. Both the presence of antigen and the amount of antigen present can be measured. For measuring proteins, for each the antigen and the presence and amount (abundance) of the protein can be determined or measured. Measuring the quantity of antigen can be achieved by a variety of methods. One of the most common is to label either the antigen or antibody with a detectable label.

An “enzyme linked immunosorbent assay (ELISA)” is type of immunoassay used to test for antigens (for example, proteins present in a sample, such as a biological sample). A “competitive radioimmunoassay (RIA)” is another type of immunoassay used to test for antigens. A “lateral flow immunochromatographic (LFI)” assay is another type of immunoassay used to test for antigens.

Label: A detectable compound or composition that is conjugated directly or indirectly to another molecule, such as an antibody or a protein, to facilitate detection of that molecule. Specific, non-limiting examples of labels include fluorescent tags, enzymatic linkages (such as horseradish peroxidase), radioactive isotopes (for example ¹⁴C, ³²P, ¹²⁵I, ³H isotopes and the like) and particles such as colloidal gold. In some examples a protein, such as a protein associated with a particular infection, is labeled with a radioactive isotope, such as ¹⁴C, ³²P, ¹²⁵I, ³H isotope. In some examples an antibody that specifically binds the protein is labeled. Methods for labeling and guidance in the choice of labels appropriate for various purposes are discussed for example in Sambrook et al. (Molecular Cloning: A Laboratory Manual, Cold Spring Harbor, N. Y., 1989) and Ausubel et al. (In Current Protocols in Molecular Biology, John Wiley & Sons, New York, 1998), Harlow & Lane (Antibodies, A Laboratory Manual, Cold Spring Harbor Publications, New York, 1988).

Sequence identity: As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

Variants: sequences derived by deletion (so-called truncation) or addition of one or more amino acids to the N-terminal and/or C-terminal end, and/or addition of one or more bases to the 5′ or 3′ end of the nucleic acid sequence; deletion or addition of one or more amino acids/nucleic acids at one or more sites in the sequence; or substitution of one or more amino acids/nucleic acids at one or more sites in the sequence. The antibodies and antibody fragments described herein may be altered in various ways including amino acid substitutions, deletions, truncations, and insertions. Methods for such manipulations are generally known in the art. For example, amino acid sequence variants of the enzyme can be prepared by mutations in the DNA. Methods for mutagenesis and nucleotide sequence alterations are well known in the art. The substitution may be a conserved substitution. A “conserved substitution” is a substitution of an amino acid with another amino acid having a similar side chain. A conserved substitution would be a substitution with an amino acid that makes the smallest change possible in the charge of the amino acid or size of the side chain of the amino acid (alternatively, in the size, charge or kind of chemical group within the side chain) such that the overall enzyme retains its spatial conformation but has altered biological activity. For example, common conserved changes might be Asp to Glu, Asn or Gln; His to Lys, Arg or Phe; Asn to Gln, Asp or Glu and Ser to Cys, Thr or Gly. Alanine is commonly used to substitute for other amino acids. The 20 essential amino acids can be grouped as follows: alanine, valine, leucine, isoleucine, proline, phenylalanine, tryptophan and methionine having nonpolar side chains; glycine, serine, threonine, cystine, tyrosine, asparagine and glutamine having uncharged polar side chains; aspartate and glutamate having acidic side chains; and lysine, arginine, and histidine having basic side chains.

### General Description

The inventors analyzed sera samples from 12 FTD cases with TAR DNA-binding protein 43 (TDP-43) proteinopathy and 12 FTD cases with tau proteinopathy using a panel of 14 scFvs and a sensitive phage capture ELISA system. Seven of the scFvs bound select protein variants of TDP-43, three bound select oligomeric variants of beta-amyloid, two bound select oligomeric variants of tau and two bound select oligomeric variants of alpha-synuclein. Not every scFv recognized every case but all 24 FTD-TDP and FTD-Tau cases were selected with the panel. Some of the scFvs were significantly more reactive with the FTD-Tau cases compared to FTD-TDP cases including the TDP-43 reactive scFvs AD-TDP2 and AD-TDP3 and the oligomeric tau reactive scFv F9T. The FTD-TDP1 and FTD-TDP2 reactive scFvs were significantly more reactive with both the FTD-TDP and FTD-Tau cases compared to the controls. Most surprising of all was that the oligomeric beta-amyloid reactive scFvs A4 and E1 displayed strong sensitivity and specificity for all 24 cases indicating a role of beta-amyloid oligomers in FTD pathology and a significant target during therapy. FTD-TDP cases generated excellent negative correlations between MMSE scores and select protein variants while FTD-Tau cases produced excellent negative correlations between UPDRS motor scores and select protein variants. Similarly, excellent negative correlations were detected between select protein variants levels and tau tangle levels in the FTD-Tau cases. APOE genotype did not seem to influence protein variants levels, in particular beta-amyloid oligomer levels like in Alzheimer's disease (AD). No major differences were detected between males and females in the FTD-TDP cases and only slight differences in the FTD-Tau cases. Overall, diverse binding patterns exist between FTD-TDP and FTD-Tau cases as well as across the cases present within each subgroup. These results indicate that FTD cases benefit from personalized diagnostic tests and therapeutics strategies. Based on the results of the current study, the disclosed scFvs serve as excellent indicators of progressing pathology including that of oligomeric beta-amyloid and also can function therapeutically to further prevent disease advancement.

As such, disclosed herein antibodies, antibody fragments, binding agents, and compositions that specifically recognize protein variant biomarkers associated with frontotemporal dementia (FTD), and methods of use, including diagnosing, monitoring and treating frontotemporal dementia. In some embodiments, a single chain antibody molecule has an amino acid sequence as provided below:

6E target: a-syn
(SEQ ID NO: 1)
MAEVQLLESGGGLVQPGGSLRLSCAASGFTFSSYA
MSWVRQAPGKGLEWVSYIASGGDTTNYADSVKGRF
TISRDNSKNTLYLQMNSLRAEDTAVYYCAKGASAF
DYWGQGTLVTVSSGGGGSGGGGSGGGGSTDIQMTQ
SPSSLSASVGDRVTITCRASQSISSYLNWYQQKPG
KAPKLLIYAASYLQSGVPSRFSGSGSGTDFTLTIS
SLQPEDFATYYCQQSSNDPYTFGQGTKVEIKRAAA
HHHHHHGAAEQKLISEEDLNGAA*
10H target: a-syn
(SEQ ID NO: 2)
MAEVQLLESGGGLVQPGGSLRLSCAASGFTFSSYA
MSWVRQAPGKGLEWVSNISSAGKGLEWVSSIDDSG
ASTYYADSVKGRFTISRDNSKNTLYLQMNSLRAED
TAVYYCAKDSASFDYWGQGTLVTVSSGGGGSGGGG
SGGGGSTDIQMTQSPSSLSASVGDRVTITCRASQS
ISSYLNWYQQKPGKAPKLLIYTASSLQSGVPSRFS
GSGSGTDFTLTISSLQPEDFATYYCQQSAASPSTF
GQGTKVEIKRAAAHHHHHHGAAEQKLISEEDLNGA
A
a4 target: aBeta
(SEQ ID NO: 3)
MAEVQLLESGGGLVQPGGSLRLSCAASGFTFSSYP
MSWVRQAPGKGLEWVSAIQHTGAPTTYADSVKGRF
TISRDNSKNTLYLQMNSLRAEDTAVYYCAKAFPPF
DYWGQGTLVTVSSGGGGSGGGGSGGGGSTDIQMTQ
SPSSLSASVGDRVTITCRASQSISSYLNWYQQKPG
KAPKLLIYSASSLQSGVPSRFSGSGSGTDFTLTIS
SLQPEDFATYYCQQRETGPKAFGQGTKVEIKRAAA
HHHHHHGAAEQKLISEEDLNGAA
E1 target: aBeta
(SEQ ID NO: 4)
ETVIMKYLLPTAAAGLLLLAAQPAMAEVQLLESGG
GLVQPGGSLRLSCAASGFTFSSYAMSWVRQAPGKG
LEWVSSIQPEGRRTAYVDSVKGRFTISRDNSKNTL
YLQMNSLRAEDTAVYYCAKPPERFDYWGQGTLVTV
SSGGGGSGGGGSGGGGSTDIQMTQSPSSLSASVGD
RVTITCRASQSISSYLNWYQQKPGKAPKLLIYAAS
SLQSGVPSRFSGSGSGTDFTLTISSLQPEDFATYY
CQQSYSTPNTFGQGTKVEIKRAAAHHHHHHGAAEQ
KLISEEDLNGAA-
D5 target: a-syn
(SEQ ID NO: 5)
MAEVQLLESGGGLVQPGGSLRLSCAASGFTFSSYA
MSWVRQAPGKGLEWVSSIGQKGGGTQYADSVKGRF
TISRDNSKNTLYLQMNSLRAEDTAVYYCAKHFENF
DYWGQGTLVTVSSGGGGSGGGGSGGGGSTDIQMTQ
SPSSLSASVGDRVTITCRASQSISSYLNWYQQKPG
KAPKLLIYAASHLQSGVPSRFSGSGSGTDFTLTIS
SLQPEDFATYYCQQTRRPPSTFGQGTKVEIKRAAA
HHHHHHGAAEQKLISEEDLNGAA
D11c target: tau
(SEQ ID NO: 6)
MKYLLPTAAAGLLLLAAQPAMAQVQLVESGGGLVQ
PGGSLRLSCAASGFTFSSYAMSWVRQAPGKGLEWV
SAISGSGGSTYYADSVKGRFTISRDNSKNTLYLQM
NSLRAEDTAVYYCARGGDYGSGDYWGQGTLVTVSS
GGGGSGGGGSGGGGSNFMLTQDPAVSVALGQTVRI
TCQGDSLRSYYASWYQQKPGQAPLLVIYGKNIRPS
GIPDRFSGSSSGNSASLTITGAQAEDEADYYCHSR
DSSGTHLRVFGGGTKVTVLGAAAHHHHHHGAAEQK
LISEED
F9T target: tau
(SEQ ID NO: 7)
MAQVQLQESGGGVVQPGRSLRLSCAASGFTFSTSG
MHWVRQAPGKGLEWVAFILHDGSDKYYADSVKGRF
TISRDNSKNTLYLQMNSLRAEDTAIYYCAKSQREL
LGAEYLQNWGQGTLVTVSSGGGGSGGGGSGGGGSQ
SALTQPASVSGSPGQSITISCTGTSSDVGGYKYVS
WYQQHPGKAPKVMIYDVSNRPSGVSNRFSGSKSGN
TASLTISGLQAEDEADYYCSSYTSSSTLVFGGGTK
VTVLGAAAHHHHHHGAAEQKLISEEDLNGAA*
AD-TDP1 target: TDP
(SEQ ID NO: 8)
MAEVQLLESGGGLVQPGGSLRLSCAASGFTFSSYA
MSWVRQAPGKGLEWVSDIGGDGYNTSYADSVKGRF
TISRDNSKNTLYLQMNSLRAEDTAVYYCAKSYTAF
DYWGQGTLVTVSSGGGGSGGGGSGGGGSTDIQMTQ
SPSSLSASVGDRVTITCRASQSISSYLNWYQQKPG
KAPKLLIYSASGLQSGVPSRFSGSGSGTDFTLTIS
SLQPEDFATYYCQQDTNGPSTFGQGTKVEIKRAA
AD-TDP2 target: TDP
(SEQ ID NO: 9)
MAEVFDYWGQGTLVTVSSGGGGSGGGGSGGGGSTD
IQMTQSPSSLSASVGDRVTITCRASQSISSYLNWY
QQKPGKAPKLLIYYASTLQSGVPSRFSGSGSGTDF
TLTISSLQPEDFATYYCQQNYNSPYTFGQGTKVEI
KRAA
AD-TDP3 target: TDP
(SEQ ID NO: 10)
MAEVQLLESGGGLVQPGGSLRLSCAASGFTFSSYA
MSWVRQAPGKGLEWVSTINNSGTSTNYADSVKGRF
TISRDNSKNTLYLQMNSLRAEDTAVYYCAKSTNYF
DYWGQGTLVTVSSGGGGSGGGGSGGGGSTDIQMTQ
SPSSLSASVGDRVTITCRASQSISSYLNWYQQKPG
KAPKLLIYAASSLQSGVPSRFSGSGSGTDFTLTIS
SLQPEDFATYYCQQNAADPTTFGQGTKVEIKRAA
als-tdp6 target: TDP
(SEQ ID NO: 11)
MAEVQLLESGGGLVQPGGSLRLSCAASGFTFSSYA
MSWVRQAPGKGLEWVSSIASSGDDTNYADSVKGRF
TISRDNSKNTLYLQMNSLRAEDTAVYYCAKTASSF
DYWGQGTLVTVSSGGGGSGGGGSGGGGSTDIQMTQ
SPSSLSASVGDRVTITCRASQSISSYLNWYQQKPG
KAPKLLIYSASSLQSGVPSRFSGSGSGTDFTLTIS
SLQPEDFATYYCQQTNGNPNTFGQGTKVEIKRAA
ALS-TDP-10 target: TDP
(SEQ ID NO: 12)
MAEVQLLESGGGLVQPGGSLRLSCAASGFTFSSYA
MSWVRQAPGKGLEWVSTIRHAGQSTDIQMTQSPSS
LSASVGDRVTITCRASQSISSYLNWYQQKPGKAPK
LLIYMASRLQSGVPSRFSGSGSGTDFTLTISSLQP
EDFATYYCQQQRTKPPTFGQGTKVEIKRAA
ALS-TDP-14 target: TDP
(SEQ ID NO: 13)
MAEVQLLESGGGLVQPGGSLRLSCAASGFTFSSYA
MSWVRQAPGKGLEWVSAIGASGNATAYADSVKGRF
TISRDNSKNTLYLQMNSLRAEDTAVYYCAKSTTDF
DYWGQGTLVTVSSGGGGSGGGGSGGGGSTDIQMTQ
SPSSLSASVGDRVTITCRASQSISSYLNWYQQKPG
KAPKLLIYNASGLQSGVPSRFSGSGSGTDFTLTIS
SLQPEDFATYYCQQAANYPTTFGQGTKVEIKRAA
ALS-TDP-9 target: TDP
(SEQ ID NO: 14)
MAEVQLLESGGGLVQPGGSLRLSCAASGFTFSSYA
MSWVRQAPGKGLEWVSSIYSDGGATSYADSVKGRF
TISRDNSKNTLYLQMNSLRAEDTAVYYCAKATATF
DYWGQGTLVTVSSGGGGSGGGGSGGGGSTDIQMTQ
SPSSLSASVGDRVTITCRASQSISSYLNWYQQKPG
KAPKLLIYSASALQSGVPSRFSGSGSGTDFTLTIS
SLQPEDFATYYCQQSSTSPSTFGQGTKVEIKRAA
ALS-TDP-12 target: TDP
(SEQ ID NO: 15)
MAEVQLLESGGGLVQPGGSLRLSCAASGFTFSSYA
MSWVRQAPGKGLEWVSNIAGNGSYTYYADSVKGRF
TISRDNSKNTLYLQMNSLRAEDTAVYYCAKDDAAF
DYWGQGTLVTVSSGGGGSGGGGSGGGGSTDIQMTQ
SPSSLSASVGDRVTITCRASQSISSYLNWYQQKPG
KAPKLLIYSASYLQSGVPSRFSGSGSGTDFTLTIS
SLQPEDFATYYCQQSATTPNTFGQGTKVEIKRAA
VL not SwFTD
FTD-TDP1 target: TDP
(SEQ ID NO: 16)
MKYLLPTAAAGLLLLAAQPAMAEVQLLESGGGLVQ
PGGSLRLSCAASGFTFSSYAMSWVRQAPGKGLEWV
SAISSNGSGTQYADSVKGRFTISRDNSKNTLYLQM
NSLRAEDTAVYYCAKYGSTFDYWGQGTLVTVSSGG
GGSGGGGSGGGGSTDIQMTQSPSSLSASVGDRVTI
TCRASQSISSYLNWYQQKPGKAPKLLIYAASHLQS
GVPSRFSGSGSGTDFTLTISSLQPEDFATYYCQQS
DDAPTTFGQGTKVEIKRAAAHHHHHHGAAEQKLIS
EED
FTD-TDP2 target: TDP
(SEQ ID NO: 17)
MAEVQLLESGGGLVQPGGSLRLSCAASGFTFSSYA
MSWVRQAPGKGLEWVSAIASAGCTTQYADSVKGRF
TISRDNSKNTLYLQMNSLRAEDTAVYYCAKCNATF
DYWGQGTLVTVSSGGGGSGGGGSGGGGSTDIQMTQ
SPSSLSASVGDRVTITCRASQSISIDLNWYQQKPG
KAPKLLIYAASHLQSGVPSRFSGSGSGTDFTLTIS
SLQPEDFATYYCQQSADSPYTFGQGTKVEIKRAAA
HHHHHHGAAEQKLISEED
FTD-TDP3 target: TDP
(SEQ ID NO: 18)
MAEVQLLESGGGLVQPGGSLRLSCAASGFTFSSYA
MSWVRQAPGKGLEWVSSISAAGDYTTYADSVKGRF
TISRDNSKNTLYLQMNSLRAEDTAVYYCAKDSTSF
DYWGQGTLVTVSSGGGGSGGGGSGGGGSTDIQMTQ
SPSSLSASVGDRVTITCRASQSISSYLNWYQQKPG
KAPKLLIYAASNLQSGVPSRFSGSGSGTDFTLTIS
SLQPEDFATYYCQQTAAYPTTFGQGTKVEIKRAAA
HHHHHHGAAEQKLISEED
FTD-TDP4 target: TDP
(SEQ ID NO: 19)
MAEVQLLESGGGLVQPGGSLRLSCAASGFTFSSYA
MSWVRQAPGKGLEWVSNITAAGSDTYYADSVKGRF
TISRDNSKNTLYLQMNSLRAEDTAVYYCAKNSTYF
DYWGQGTLVTVSSGGGGSGGGGSGGGGSTDIQMTQ
SPSSLSASVGDRVTITCRASQSISSYLNWYQQKPG
KAPKLLIYNASDLQSGVPSRFSGSGSGTDFTLTIS
SLQPEDFATYYCQQASGDPDTFGQGTKVEIKRAAA
HHHHHHGAAEQKLISEED
FTD-TDP5 target: TDP
(SEQ ID NO: 20)
MAEVQLLESGGGLVQPGGSLRLSCAASGFTFSSYA
MSWVRQAPGKGLEWVSDISGNGGATNYADSVKGRF
TISRDNSKNTLYLQMNSLRAEDTAVYYCAKATTGF
DYWGQGTLVTVSSGGGGSGGGGSGGGGSTDIQMTQ
SPSSLSASVGDRVTITCRASQSISSYLNWYQQKPG
KAPKLLIYNASYLQSGVPSRFSGSGSGTDFTLTIS
SLQPEDFATYYCQQSSTTPSTFGQGTKVEIKRAAA
HHHHHHGAAEQKLISEED

In some embodiments, antibody and/or fragment thereof comprises one or more light and/or heavy chain complementary determining regions (CDRs), such as 1, 2, 3, 4, 5, or 6 as disclosed in Table 3 below.

TABLE 3

| | | | | | | | | Chothia | |
|---|---|---|---|---|---|---|---|---|---|
| | | KABAT | | | | | | difference | |
| | | L24- | L50- | L89- | KABAT | | H95- | H26- | H52- |
| | | L34 | L56 | L97 | H31-35 | H50-h65 | h102 | H32 | H56 |
| | | CDR-L1 | CDR-L2 | CDR-L3 | CDR-H1 | CDR-H2 | CDR-H3 | CDR-H1 | CDR-H2 |
| | | (SEQ | (SEQ | (SEQ | (SEQ | (SEQ | (SEQ | (SEQ | (SEQ |
| Name | ANTIGEN | ID NO) | ID NO:) | ID NO:) | ID NO:) | ID NO:) | ID NO:) | ID NO:) | ID NO:) |
| 6E | a-syn | RAS | AASYL | QQSSN | SYAMS | YIASGGD | GASAF | GFTFS | ASGG |
| | | QSIS | QS (24) | DPYT | (61) | TTNYADS | DY (83) | SY | DT |
| | | SYLN | | (41) | | VKG (64) | | (101) | (103) |
| | | (21) | | | | | | | |
| 10H | a-syn | RAS | TASSL | QQSA | SYAMS | NISSAGK | DSASF | | |
| | | QSIS | QS (25) | ASPST | (61) | GLEWVSS | DY (84) | | |
| | | SYL | | (42) | | IDDSGAS | | | |
| | | N | | | | TYYADSV | | | |
| | | (21) | | | | KG (65) | | | |
| a4 | aBeta | RAS | SASSL | QQRET | SYMPS | AIQHTGA | AFPPF | GFTFS | QHTG |
| | | QSIS | QS (26) | GPKA | (62) | PTTYADS | DY (85) | SY | AP |
| | | SYLN | | (43) | | VKG (66) | | (101) | (104) |
| | | (21) | | | | | | | |
| E1 | aBeta | RAS | AASSL | QQSYS | SYAMS | SIQPEGRR | PPERF | GFTFS | QPEGR |
| | | QSIS | QS (27) | TPNT | (61) | TAYVDSV | DY (86) | SY | R (105) |
| | | SYLN | | (44) | | KG (67) | | (101) | |
| | | (21) | | | | | | | |
| D5 | a-syn | RAS | AASHL | QQTRR | SYAMS | SIGQKGG | HFENF | GFTFS | GQKG |
| | | QSIS | QS (28) | PPST | (61) | GTQYADS | DY (87) | SY | GG |
| | | SYLN | | (45) | | VKG (68) | | (101) | (106) |
| | | (21) | | | | | | | |
| D11c | tau | QGD | GKNIR | HSRDS | SYAMS | AISGSGG | GGDY | GFTFS | SGSGG |
| | | SLRS | PS (29) | SGTHL | (61) | STYYADS | GSGDY | SY | S (107) |
| | | YYA | | RV (46) | | VKG (69) | (88) | (101) | |
| | | S (22) | | | | | | | |
| F9T | tau | TGTS | DVSNR | SSYTS | TSGMH | FILHDGS | SQREL | GFTFS | LHDGS |
| | | SDV | PS (30) | SSTLV | (63) | DKYYAD | LGAEY | TS | D(108) |
| | | GGY | | (47) | | SVKG (70) | LQN | (102) | |
| | | KYV | | | | | (89) | | |
| | | S | | | | | | | |
| | | (23) | | | | | | | |
| AD- | TDP | RAS | SASGL | QQDT | SYAMS | DIGGDGY | SYTAF | GFTFS | GGDG |
| TDP1 | | QSIS | QS (31) | NGPST | (61) | NTSYADS | DY (90) | SY | YN |
| | | SYLN | | (48) | | VKG (71) | | (101) | (109) |
| | | (21) | | | | | | | |
| AD- | TDP | RAS | YASTL | QQNY | N/A | N/A | N/A | N/A | N/A |
| TDP2 | | QSIS | QS (32) | NSPYT | | | | | |
| | | SYLN | | (49) | | | | | |
| | | (21) | | | | | | | |
| AD- | TDP | RAS | AASSL | QQNA | SYAMS | TINNSGTS | STNYF | | |
| TDP3 | | QSIS | QS (27) | ADPTT | (61) | TNYADSV | DY (91) | | |
| | | SYLN | | (50) | | KG (72) | | | |
| | | (21) | | | | | | | |
| als- | TDP | RAS | SASSL | QQTN | SYAMS | SIASSGD | TASSF | GFTFS | ASSGD |
| tdp6 | | QSIS | QS (26) | GNPNT | (61) | DTNYADS | DY (92) | SY | D(110) |
| | | SYLN | | (51) | | VKG (73) | | (101) | |
| | | (21) | | | | | | | |
| ALS- | TDP | RAS | MASR | QQQR | Syams | TIRHAGQ | N/A | GFTFS | RHAG |
| TDP- | | QSIS | LQS | TKPPT | (61) | (74) | | SY | Q (111) |
| 10 | | SYLN | (33) | (52) | | | | (101) | |
| | | (21) | | | | | | | |
| ALS- | TDP | RAS | NASGL | QQAA | SYAMS | AIGASGN | STTDF | GFTFS | GASG |
| TDP- | | QSIS | QS (34) | NYPTT | (61) | ATAYADS | DY (93) | SY | NA |
| 14 | | SYLN | | (53) | | VKG (75) | | (101) | (112) |
| | | (21) | | | | | | | |
| ALS- | TDP | RAS | SASAL | QQSST | SYAMS | SIYSDGG | ATATF | GFTFS | YSDG |
| TDP-9 | | QSIS | QS (35) | SPST | (61) | ATSYADS | DY (94) | SY | GA |
| | | SYLN | | (54) | | VKG (76) | | (101) | (113) |
| | | (21) | | | | | | | |
| ALS- | TDP | RAS | SASYL | QQSAT | SYAMS | NIAGNGS | DDAAF | GFTFS | AGNG |
| TDP- | | QSIS | QS (36) | TPNT | (61) | YTYYADS | DY (95) | SY | SY |
| 12 | | SYLN | | (55) | | VKG (77) | | (101) | (114) |
| | | (21) | | | | | | | |

VL not SwFTD

| | | | | | | | | | |
|---|---|---|---|---|---|---|---|---|---|
| FTD- | TDP | RAS | AASHL | QQSD | SYAMS | AISSNGS | YGSTF | GFTFS | SSNGS |
| TDP1 | | QSIS | QS (28) | DAPTT | (61) | GTQYADS | DY (96) | SY | G (115) |
| | | SYLN | | (56) | | VKG (78) | | (101) | |
| | | (21) | | | | | | | |
| FTD- | TDP | RAS | AASHL | QQSA | SYAMS | AIASAGC | CNATF | GFTFS | ASAG |
| TDP2 | | QSIS | QS (28) | DSPYT | (61) | TTQYADS | DY (97) | SY | CT |
| | | IDLN | | (57) | | VKG (79) | | (101) | (116) |
| | | (37) | | | | | | | |
| FTD- | TDP | RAS | AASNL | QQTA | SYAMS | SISAAGD | DSTSF | GFTFS | SAAG |
| TDP3 | | QSIS | QS (38) | AYPTT | (61) | YTTYADS | DY (98) | SY | DY |
| | | SYLN | | (58) | | VKG (80) | | (101) | (117) |
| | | (21) | | | | | | | |
| FTD- | TDP | RAS | NASDL | QQAS | SYAMS | NITAAGS | NSTYF | GFTFS | TAAGS |
| TDP4 | | QSIS | QS (39) | GDPDT | (61) | DTYYADS | DY (99) | SY | D (118) |
| | | SYLN | | (59) | | VKG (81) | | (101) | |
| | | (21) | | | | | | | |
| FTD- | TDP | RAS | NASYL | QQSST | SYAMS | DISGNGG | ATTGF | GFTFS | SGNG |
| TDP5 | | QSIS | QS (40) | TPST | (61) | ATNYADS | DY | SY | GA |
| | | SYLN | | (60) | | VKG (82) | (100) | (101) | (119) |
| | | (21) | | | | | | | |

An amino acid sequence of an antibody or antibody fragment or variant thereof described herein or a nucleic acid sequence or variant thereof encoding such an amino acid sequence, is a sequence that is substantially similar to those disclosed in Table 3 or listed above. Variant amino acid and nucleic acid sequences include synthetically derived amino acid and nucleic acid sequences, or recombinantly derived amino acid or nucleic acid sequences. Generally, nucleic acid or amino acid sequences of the invention will have at least 40 to 100% sequence identity to the disclosed sequences provided herein. In certain embodiments, the nucleic acid or amino acid sequences of the invention will have at least 50, 60, 70%, 71%, 72%, 73%, 74%, 75%, 76%, 77%, 78% 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the sequences provided herein.

These disclosed compounds may be used in diagnostic as well therapeutic applications and may be either administered to patients or used on patient tissue samples. In some embodiments, the compositions of the present invention may be used for in vivo imaging of target morphologies of molecules associated with FTD as compared with those observed in normal neurological tissue. As such, the nanobody compositions of the invention may be used to detect and quantitate the FTD-molecules disclosed herein. In another embodiment, the compounds may be used in the treatment or prophylaxis of neurodegenerative disorders. Also provided herein are methods of allowing the compound to distribute into the brain tissue, and imaging the brain tissue, wherein an increase in binding of the compound to the brain tissue compared to a normal control level of binding indicates that the mammal is suffering from or is at risk of developing a neurodegenerative disease, such FTD.

The methods of the present invention can be used to provide early stage diagnosis of neurodegenerative conditions and diseases, such as FTD. In some examples, a subject is diagnosis with FTD when an alteration, in one or more FTD-associated molecules is detected. An alteration can be an increase or decrease in an FTD-associated molecule activity, expression level and/or combination thereof. In some examples, an alteration is an increase in an FTD-associated molecule expression level, such as at least a 2-fold, 3-fold, 4-fold or more increase in expression of the FTD-associated molecule as compared to a control (such as a subject that does not have FTD). In some examples, an alteration is an increase in an FTD-associated molecule expression level, such as at least a 5% increase, 10%, increase, 15% increase, 20% increase, 25% increase, 30% increase, 35% increase, 40% increase, 45% increase, 50% or more increase in expression of the FTD-associated molecule as compared to a control (such as a subject that does not have FTD). In some examples, an increase in an FTD-associated molecule predicts with a specificity and/or sensitivity of at least 90%, such as at least 95%, at least 98%, including 90%, 91%, 92%, 93%, 94%, 95%, 96%, 97%, 98%, 99% or 100% that the subject has FTD. The compositions disclose comprising these antibodies and antibody fragments may be used to identify molecules associated with FTD in a biological sample from a patient to be tested for a neurodegenerative disease, wherein the presence of FTD associated with FTD in the sample is indicative that the patient has or is likely to develop FTD. In certain embodiments, the assay format that is used may be any assay format that typically employs antibody compositions. Thus, for example, the biological sample may be examined using immunohistology techniques, ELISA, Western Blotting, and the like.

For purposes of the diagnostic methods of the invention, the compositions of the invention may be conjugated to a detecting reagent that facilitates detection of the antibody or fragment thereof, such as an scFv. For example, example, the detecting reagent may be a direct label or an indirect label. The labels can be directly attached to or incorporated into the detection reagent by chemical or recombinant methods.

In one embodiment, a label is coupled to the antibody or fragment thereof, such as a scFv through a chemical linker. Linker domains are typically polypeptide sequences, such as poly gly sequences of between about 5 and 200 amino acids. In some embodiments, proline residues are incorporated into the linker to prevent the formation of significant secondary structural elements by the linker. In certain embodiments, linkers are flexible amino acid subsequences that are synthesized as part of a recombinant fusion protein comprising the RNA recognition domain. In one embodiment, the flexible linker is an amino acid subsequence that includes a proline, such as Gly(x)-Pro-Gly(x) where x is a number between about 3 and about 100. In other embodiments, a chemical linker is used to connect synthetically or recombinantly produced recognition and labeling domain subsequences. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

The detectable labels can be used in the assays of the present invention to diagnose a neurodegenerative disease, such as FTD, these labels are attached to the antibodies or fragment thereof, such as scFvs of the invention, can be primary labels (where the label comprises an element that is detected directly or that produces a directly detectable element) or secondary labels (where the detected label binds to a primary label, e.g., as is common in immunological labeling). An introduction to labels, labeling procedures and detection of labels is found in Polak and Van Noorden (1997) Introduction to Immunocytochemistry, 2nd ed., Springer Verlag, N.Y. and in Haugland (1996) Handbook of Fluorescent Probes and Research Chemicals, a combined handbook and catalogue Published by Molecular Probes, Inc., Eugene, Oreg. Patents that described the use of such labels include U.S. Pat. Nos. 3,817,837; 3,850,752; 3,939,350; 3,996,345; 4,277,437; 4,275,149; and 4,366,241.

Primary and secondary labels can include undetected elements as well as detected elements. Useful primary and secondary labels in the present invention can include spectral labels such as green fluorescent protein, fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., 3H, 125I, 35S, 14C, 32P, 33P, etc.), enzymes (e.g., horse radish peroxidase, alkaline phosphatase etc.), spectral calorimetric labels such as colloidal gold or colored glass or plastic (e.g., polystyrene, polypropylene, latex, etc.) beads. The label can be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

Exemplary labels that can be used include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate (kits available from Life Technologies/Gibco BRL, and Boehringer-Mannheim)); 3) fluorescence using, e.g., an enzyme such as alkaline phosphatase, together with the substrate AttoPhos (Amersham) or other substrates that produce fluorescent products, 4) fluorescence (e.g., using Cy-5 (Amersham), fluorescein, and other fluorescent tags); 5) radioactivity. Other methods for labeling and detection will be readily apparent to one skilled in the art.

Where the antibody or fragment thereof, such as an scFv,-based compositions of the invention are contemplated to be used in a clinical setting, the labels are preferably non-radioactive and readily detected without the necessity of sophisticated instrumentation. In certain embodiments, detection of the labels will yield a visible signal that is immediately discernable upon visual inspection. One example of detectable secondary labeling strategies uses an antibody that recognizes a disclosed FTD-associated molecule in which the antibody is linked to an enzyme (typically by recombinant or covalent chemical bonding). The antibody is detected when the enzyme reacts with its substrate, producing a detectable product. In certain embodiments, enzymes that can be conjugated to detection reagents of the invention include, e.g., β-galactosidase, luciferase, horse radish peroxidase, and alkaline phosphatase. The chemiluminescent substrate for luciferase is luciferin. One embodiment of a fluorescent substrate for β-galactosidase is 4-methylumbelliferyl-β-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl) spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ABTS), 5-aminosalicylic acid (5AS), o-dianisidine, and o-phenylenediamine (OPD), which are detected with a spectrophotometer, and 3,3,5,5′-tetramethylbenzidine (TMB), 3,3′ diaminobenzidine (DAB), 3-amino-9-ethylcarbazole (AEC), and 4-chloro-1-naphthol (4CIN), which are detected visually. Other suitable substrates are known to those skilled in the art. The enzyme-substrate reaction and product detection are performed according to standard procedures known to those skilled in the art and kits for performing enzyme immunoassays are available as described above.

The presence of a label can be detected by inspection, or a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling moieties is digitized for subsequent computer analysis.

As noted herein throughout the antibodies or fragment thereof, such as scFvs, of the invention are targeted specifically to FTD-associated molecules. As such, the scFvs of the invention also may be used to specifically target therapeutic compositions to the sites of aggregation of the FTD-associated molecules. In this embodiment, any therapeutic agent typically used for the treatment of these diseases, may be conjugated to scFvs in order to achieve a targeted delivery of that therapeutic agent.

The antibodies or fragment thereof, such as scFvs, compositions of the invention can be used in any diagnostic assay format to determine the presence of FTD. A variety of immunodetection methods are contemplated for this embodiment. Such immunodetection methods include enzyme linked immunosorbent assay (ELISA), radioimmunoassay (RIA), immunoradiometric assay, fluoroimmunoassay, chemiluminescent assay, bioluminescent assay, and Western blot, though several others are well known to those of ordinary skill. The steps of various useful immunodetection methods have been described in the scientific literature.

In general, the immunobinding methods include obtaining a sample suspected of containing a protein, polypeptide and/or peptide, and contacting the sample with a first antibody, monoclonal or polyclonal, in accordance with the present invention, as the case may be, under conditions effective to allow the formation of immunocomplexes.

The immunobinding methods include methods for detecting and quantifying the amount of the FTD-associated molecules in a sample and the detection and quantification of any immune complexes formed during the binding process. Here, one would obtain a sample suspected of containing a disclosed FTD-associated molecule, and contact the sample with an antibody fragment of the invention, and then detect and quantify the amount of immune complexes formed under the specific conditions.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those scFv molecules specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

As noted above, an antibody or fragment thereof, such as an scFv, of the invention may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody, that has binding affinity for the scFV is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first step biotinylated, monoclonal or polyclonal antibody (in the present example a scFv of the invention) is used to detect the target antigen(s), and a second step antibody is then used to detect the biotin attached to the complexed nanobody. In this method the sample to be tested is first incubated in a solution containing the first step nanobody. If the target antigen is present, some of the nanobody binds to the antigen to form a biotinylated nanobody/antigen complex. The nanobody/antigen complex is then amplified by incubation in successive solutions of streptavidin (or avidin), biotinylated DNA, and/or complementary biotinylated DNA, with each step adding additional biotin sites to the nanobody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the method described above up to the incubation with biotinylated DNA. However, instead of using multiple rounds streptavidin and of biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

The diagnostic assay format that may be used in the present invention could take any conventional format such as ELISA or other platforms such as luminex or biosensors. The present invention shows the sequence of certain exemplary DNA sequences for binding agents specific for FTD-associated molecules. These sequences can readily be modified to facilitate diagnostic assays, for example a tag (such as GFP) can be added to these scFvs to increase sensitivity. In one exemplary ELISA, antibodies are immobilized onto a selected surface exhibiting protein affinity, such as a well in a polystyrene microtiter plate. Then, a test composition suspected of containing FTD-associated molecules, such as a clinical sample (e.g., a biological sample obtained from the subject), is added to the wells. After binding and/or washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and/or then contacted with binding agents (e.g., scFvs of the invention). After binding and/or washing to remove non-specifically bound immune complexes, the bound anti-binding agents are detected. Where the initial binding agents are linked to a detectable label, the immune complexes may be detected directly. Again, the immune complexes may be detected using a second antibody that has binding affinity for the first binding agents, with the second antibody being linked to a detectable label.

Another ELISA in which the antigens are immobilized, involves the use of antibody competition in the detection. In this ELISA, labeled antibodies (or nanobodies) against an antigen are added to the wells, allowed to bind, and/or detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes.

In coating a plate with either an FTD-associated molecule or antibody or fragment thereof, such as a scFv, of the invention, one will generally incubate the wells of the plate with a solution of the antigen or scFvs, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting FTD-associated molecule and/or scFv composition with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H2O2, in the case of peroxidase as the enzyme label. Quantification is then achieved by measuring the degree of color generated, e.g., using a visible spectra spectrophotometer.

In various aspects of the invention, it will be desirable to further subject patients to more traditional diagnostic approaches for FTD. As noted above, there are various drugs that are presently in use or under development for the treatment of FTD. The present invention contemplates the use of antibody or fragment thereof, such as a scFv, of the invention, based “diagnostic” methods to further assess the efficacy of treatments.

The present invention may involve the use of pharmaceutical compositions which comprise an agent conjugated to a scFv of the invention for delivery into a subject having FTD. Such an agent will ideally be formulated into a pharmaceutically acceptable carrier. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the therapeutic or pharmaceutical compositions is contemplated.

Changes in the profile can also represent the progression (or regression) of the disease process. Methods for monitoring the efficacy of therapeutic agents are described below. The diagnostic methods of the present disclosure are valuable tools for practicing physicians including for monitoring a subject for onset and/or advancement of FTD. The method disclosed herein can also be used to monitor the effectiveness of a therapy. Following the measurement of the expression levels of one or more of the molecules identified herein, the assay results, findings, diagnoses, predictions and/or treatment recommendations are typically recorded and communicated to technicians, physicians and/or patients, for example. In certain embodiments, computers will be used to communicate such information to interested parties, such as, patients and/or the attending physicians. Based on the measurement, the therapy administered to a subject can be modified.

In one embodiment, a diagnosis, prediction and/or treatment recommendation based on the expression level in a test subject of one or more of the FTD associated molecules disclosed herein is communicated to the subject as soon as possible after the assay is completed and the diagnosis and/or prediction is generated. The results and/or related information may be communicated to the subject by the subject's treating physician. Alternatively, the results may be communicated directly to a test subject by any means of communication, including writing, such as by providing a written report, electronic forms of communication, such as email, or telephone. Communication may be facilitated by use of a computer, such as in case of email communications. In certain embodiments, the communication containing results of a diagnostic test and/or conclusions drawn from and/or treatment recommendations based on the test, may be generated and delivered automatically to the subject using a combination of computer hardware and software which will be familiar to artisans skilled in telecommunications. One example of a healthcare-oriented communications system is described in U.S. Pat. No. 6,283,761; however, the present disclosure is not limited to methods which utilize this particular communications system. In certain embodiments of the methods of the disclosure, all or some of the method steps, including the assaying of samples, diagnosing of diseases, and communicating of assay results or diagnoses, may be carried out in diverse (e.g., foreign) jurisdictions.

In several embodiments, identification of a subject as having FTD results in the physician treating the subject, such as prescribing one or more therapeutic agents for inhibiting or delaying one or more signs and symptoms associated with FTD. In additional embodiments, the dose or dosing regimen is modified based on the information obtained using the methods disclosed herein.

The subject can be monitored while undergoing treatment using the methods described herein in order to assess the efficacy of the treatment protocol. In this manner, the length of time or the amount give to the subject can be modified based on the results obtained using the methods disclosed herein.

### Immunoassay Kits

Immunoassay kits are also disclosed herein. These kits include, in separate containers (a) monoclonal antibodies having binding specificity for the polypeptides used in the diagnosis of an FTD; and (b) and anti-antibody immunoglobulins. This immunoassay kit may be utilized for the practice of the various methods provided herein. The monoclonal antibodies and the anti-antibody immunoglobulins can be provided in an amount of about 0.001 mg to 100 grams, and more preferably about 0.01 mg to 1 gram. The anti-antibody immunoglobulin may also be a polyclonal immunoglobulin, protein A or protein G or functional fragments thereof, which may be labeled prior to use by methods known in the art. In several embodiments, the immunoassay kit includes one, two, three or four or more antibodies that specifically bind to molecules associated with FTD-associated condition or disease, such as protein antigens disclosed herein including those listed in Table 3 and/or with amino acid sequences set forth with SEQ ID NOs: 1-15. The immunoassay kit can also include one or more antibodies that specifically bind to one or more of these molecules. Thus, the kits can be used to detect one or more different molecules associated with FTD.

Immunoassays for polysaccharides and proteins differ in that a single antibody is used for both the capture and indicator roles for polysaccharides due to the presence of repeating epitopes. In contrast, two antibodies specific for distinct epitopes are required for immunoassay of proteins. Exemplary samples include biological samples obtained from subjects including, but not limited to, serum, blood and urine samples.

In one particular example, a quantitative ELISA is constructed for detection of at least one of the FTD protein antigens disclosed herein, such as those listed in Table 3. These immunoassays utilize antibodies, such as mAbs commercially available. Since a polysaccharide is a polyvalent repeating structure, a single mAb may be used for both the capture and indicator phases of an immunoassay. The only requirement is that the mAb have a sufficient affinity. A mAb with an affinity of about 0.5 μM has sufficient affinity.

### Capture Device Methods

The disclosed methods can be carried out using a sample capture device, such as a lateral flow device (for example a lateral flow test strip) that allows detection of one or more molecules, such as those described herein.

Point-of-use analytical tests have been developed for the routine identification or monitoring of health-related conditions (such as pregnancy, cancer, endocrine disorders, infectious diseases or drug abuse) using a variety of biological samples (such as urine, serum, plasma, blood, saliva). Some of the point-of-use assays are based on highly specific interactions between specific binding pairs, such as antigen/antibody, hapten/antibody, lectin/carbohydrate, apoprotein/cofactor and biotin/(strept) avidin. The assays are often performed with test strips in which a specific binding pair member is attached to a mobilizable material (such as a metal sol or beads made of latex or glass) or an immobile substrate (such as glass fibers, cellulose strips or nitrocellulose membranes). Particular examples of some of these assays are shown in U.S. Pat. Nos. 4,703,017; 4,743,560; and U.S. Pat. No. 5,073,484 (incorporated herein by reference). The test strips include a flow path from an upstream sample application area to a test site. For example, the flow path can be from a sample application area through a mobilization zone to a capture zone. The mobilization zone may contain a mobilizable marker that interacts with an analyte or analyte analog, and the capture zone contains a reagent that binds the analyte or analyte analog to detect the presence of an analyte in the sample.

Examples of migration assay devices, which usually incorporate within them reagents that have been attached to colored labels, thereby permitting visible detection of the assay results without addition of further substances are found, for example, in U.S. Pat. No. 4,770,853; WO 88/08534; and EP-A 0 299 428 (incorporated herein by reference). There are a number of commercially available lateral-flow type tests and patents disclosing methods for the detection of large analytes (MW greater than 1,000 Daltons) as the analyte flows through multiple zones on a test strip. Examples are found in U.S. Pat. No. 5,229,073 (measuring plasma lipoprotein levels), and U.S. Pat. Nos. 5,591,645; 4,168,146; 4,366,241; 4,855,240; 4,861,711; 5,120,643; European Patent No. 0296724; WO 97/06439; WO 98/36278; and WO 08/030546 (each of which are herein incorporated by reference). Multiple zone lateral flow test strips are disclosed in U.S. Pat. Nos. 5,451,504, 5,451,507, and 5,798,273 (incorporated by reference herein). U.S. Pat. No. 6,656,744 (incorporated by reference) discloses a lateral flow test strip in which a label binds to an antibody through a streptavidin-biotin interaction.

In particular examples, the methods disclosed herein include application of a biological sample (such as serum, whole blood or urine) from a human test subject to a lateral flow test device for the detection of one or more molecules (such as one or more molecules associated with FTD disease, for example, combinations of molecules as described above) in the sample. The lateral flow test device includes one or more antibodies (such as antibodies that bind one or more of the molecules associated with FTD disease) at an addressable location. In a particular example, the lateral flow test device includes antibodies that bind at least one disclosed FTD disease protein antigen. The addressable locations can be, for example, a linear array or other geometric pattern that provides diagnostic information to the user. The binding of one or more molecules in the sample to the antibodies present in the test device is detected and the presence or amount of one or more molecules in the sample of the test subject is compared to a control, wherein a change in the presence or amount of one or more molecules in the sample from the test subject as compared to the control indicates that the subject has FTD.

Devices described herein generally include a strip of absorbent material (such as a microporous membrane), which, in some instances, can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the specific binding partner(s) and/or other reagents required for the detection and/or quantification of the particular analyte being tested for, for example, one or more molecules disclosed herein. Thus, these zones can be viewed as functional sectors or functional regions within the test device.

In general, a fluid sample is introduced to the strip at the proximal end of the strip, for instance by dipping or spotting. A sample is collected or obtained using methods well known to those skilled in the art. The sample containing the particular molecules to be detected may be obtained from any biological source. Examples of biological sources include blood serum, blood plasma, urine, BALF, spinal fluid, saliva, fermentation fluid, lymph fluid, tissue culture fluid and ascites fluid of a human or animal. In a particular example, the biological source is saliva. In one particular example, the biological source is whole blood, such as a sample obtained from a finger prick. The sample may be diluted, purified, concentrated, filtered, dissolved, suspended or otherwise manipulated prior to assay to optimize the immunoassay results. The fluid migrates distally through all the functional regions of the strip. The final distribution of the fluid in the individual functional regions depends on the adsorptive capacity and the dimensions of the materials used.

Another common feature to be considered in the use of assay devices is a means to detect the formation of a complex between an analyte (such as one or more molecules described herein) and a capture reagent (such as one or more antibodies). A detector (also referred to as detector reagent) serves this purpose. A detector may be integrated into an assay device (for example included in a conjugate pad, as described below), or may be applied to the device from an external source.

A detector may be a single reagent or a series of reagents that collectively serve the detection purpose. In some instances, a detector reagent is a labeled binding partner specific for the analyte (such as a gold-conjugated antibody for a particular protein of interest, for example those described herein).

In other instances, a detector reagent collectively includes an unlabeled first binding partner specific for the analyte and a labeled second binding partner specific for the first binding partner and so forth. Thus, the detector can be a labeled antibody specific for a protein described herein. The detector can also be an unlabeled first antibody specific for the protein of interest and a labeled second antibody that specifically binds the unlabeled first antibody. In each instance, a detector reagent specifically detects bound analyte of an analyte-capture reagent complex and, therefore, a detector reagent preferably does not substantially bind to or react with the capture reagent or other components localized in the analyte capture area. Such non-specific binding or reaction of a detector may provide a false positive result. Optionally, a detector reagent can specifically recognize a positive control molecule (such as a non-specific human IgG for a labeled Protein A detector, or a labeled Protein G detector, or a labeled anti-human Ab (Fc)) that is present in a secondary capture area.

### Flow-Through Device Construction and Design

Representative flow-through assay devices are described in U.S. Pat. Nos. 4,246,339; 4,277,560; 4,632,901; 4,812,293; 4,920,046; and 5,279,935; U.S. Patent Application Publication Nos. 20030049857 and 20040241876; and WO 08/030546. A flow-through device involves a capture reagent (such as one or more antibodies) immobilized on a solid support, typically, a membrane (such as, nitrocellulose, nylon, or PVDF). Characteristics of useful membranes have been previously described; however, it is useful to note that in a flow-through assay capillary rise is not a particularly important feature of a membrane as the sample moves vertically through the membrane rather than across it as in a lateral flow assay. In a simple representative format, the membrane of a flow-through device is placed in functional or physical contact with an absorbent layer (see, e.g., description of “absorbent pad” below), which acts as a reservoir to draw a fluid sample through the membrane. Optionally, following immobilization of a capture reagent, any remaining protein-binding sites on the membrane can be blocked (either before or concurrent with sample administration) to minimize nonspecific interactions.

In operation of a flow-through device, a fluid sample (such as a bodily fluid sample) is placed in contact with the membrane. Typically, a flow-through device also includes a sample application area (or reservoir) to receive and temporarily retain a fluid sample of a desired volume. The sample passes through the membrane matrix. In this process, an analyte in the sample (such as one or more protein, for example, one or more molecules described herein) can specifically bind to the immobilized capture reagent (such as one or more antibodies). Where detection of an analyte-capture reagent complex is desired, a detector reagent (such as labeled antibodies that specifically bind one or more molecules) can be added with the sample or a solution containing a detector reagent can be added subsequent to application of the sample. If an analyte is specifically bound by capture reagent, a visual representative attributable to the particular detector reagent can be observed on the surface of the membrane. Optional wash steps can be added at any time in the process, for instance, following application of the sample, and/or following application of a detector reagent.

### Lateral Flow Device Construction and Design

Lateral flow devices are commonly known in the art. Briefly, a lateral flow device is an analytical device having as its essence a test strip, through which flows a test sample fluid that is suspected of containing an analyte of interest. The test fluid and any suspended analyte can flow along the strip to a detection zone in which the analyte (if present) interacts with a capture agent and a detection agent to indicate a presence, absence and/or quantity of the analyte.

Numerous lateral flow analytical devices have been disclosed, and include those shown in U.S. Pat. Nos. 4,168,146; 4,313,734; 4,366,241; 4,435,504; 4,775,636; 4,703,017; 4,740,468; 4,806,311; 4,806,312; 4,861,711; 4,855,240; 4,857,453; 4,861,711; 4,943,522; 4,945,042; 4,496,654; 5,001,049; 5,075,078; 5,126,241; 5,120,643; 5,451,504; 5,424,193; 5,712,172; 6,555,390; 6,258,548; 6,699,722; 6,368,876 and 7,517,699; EP 0810436; EP 0296724; WO 92/12428; WO 94/01775; WO 95/16207; WO 97/06439; WO 98/36278; and WO 08/030546, each of which is incorporated by reference. Further, there are a number of commercially available lateral flow type tests and patents disclosing methods for the detection of large analytes (MW greater than 1,000 Daltons). U.S. Pat. No. 5,229,073 describes a semiquantitative competitive immunoassay lateral flow method for measuring plasma lipoprotein levels. This method utilizes a plurality of capture zones or lines containing immobilized antibodies to bind both the labeled and free lipoprotein to give a semi-quantitative result. In addition, U.S. Pat. No. 5,591,645 provides a chromatographic test strip with at least two portions. The first portion includes a movable tracer and the second portion includes an immobilized binder capable of binding to the analyte.

Many lateral flow devices are one-step lateral flow assays in which a biological fluid is placed in a sample area on a bibulous strip (though non-bibulous materials can be used, and rendered bibulous, e.g., by applying a surfactant to the material), and allowed to migrate along the strip until the liquid comes into contact with a specific binding partner (such as an antibody) that interacts with an analyte (such as one or more molecules) in the liquid. Once the analyte interacts with the binding partner, a signal (such as a fluorescent or otherwise visible dye) indicates that the interaction has occurred. Multiple discrete binding partners (such as antibodies) can be placed on the strip (for example in parallel lines) to detect multiple analytes (such as two or more molecules) in the liquid. The test strips can also incorporate control indicators, which provide a signal that the test has adequately been performed, even if a positive signal indicating the presence (or absence) of an analyte is not seen on the strip.

The construction and design of lateral flow devices is known in the art, as described, for example, in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N. H. 03431, (603) 352-3810; both of which are incorporated herein by reference.

Lateral flow devices have a wide variety of physical formats that are equally well known in the art. Any physical format that supports and/or houses the basic components of a lateral flow device in the proper function relationship is contemplated by this disclosure.

In some embodiments, the lateral flow strip is divided into a proximal sample application pad, an intermediate test result zone, and a distal absorbent pad. The flow strip is interrupted by a conjugate pad that contains labeled conjugate (such as gold- or latex-conjugated antibody specific for the target analyte or an analyte analog). A flow path along strip passes from proximal pad, through conjugate pad, into test result zone, for eventual collection in absorbent pad. Selective binding agents are positioned on a proximal test line in the test result membrane. A control line is provided in test result zone, slightly distal to the test line. For example, in a competitive assay, the binding agent in the test line specifically binds the target analyte, while the control line less specifically binds the target analyte.

In operation of the particular embodiment of a lateral flow device, a fluid sample containing an analyte of interest, such as one or more molecules described herein (for example, protein antigens listed in Table 1 (see FIG. 7)), is applied to the sample pad. In some examples, the sample may be applied to the sample pad by dipping the end of the device containing the sample pad into the sample (such as serum or urine) or by applying the sample directly onto the sample pad (for example by placing the sample pad in the mouth of the subject). In other examples where a sample is whole blood, an optional developer fluid is added to the blood sample to cause hemolysis of the red blood cells and, in some cases, to make an appropriate dilution of the whole blood sample.

From the sample pad, the sample passes, for instance by capillary action, to the conjugate pad. In the conjugate pad, the analyte of interest, such as a protein of interest, may bind (or be bound by) a mobilized or mobilizable detector reagent, such as an antibody (such as antibody that recognizes one or more of the molecules described herein). For example, a protein analyte may bind to a labeled (e.g., gold-conjugated or colored latex particle-conjugated) antibody contained in the conjugate pad. The analyte complexed with the detector reagent may subsequently flow to the test result zone where the complex may further interact with an analyte-specific binding partner (such as an antibody that binds a particular protein, an anti-hapten antibody, or streptavidin), which is immobilized at the proximal test line. In some examples, a protein complexed with a detector reagent (such as gold-conjugated antibody) may further bind to unlabeled, oxidized antibodies immobilized at the proximal test line. The formation of a complex, which results from the accumulation of the label (e.g., gold or colored latex) in the localized region of the proximal test line is detected. The control line may contain an immobilized, detector-reagent-specific binding partner, which can bind the detector reagent in the presence or absence of the analyte. Such binding at the control line indicates proper performance of the test, even in the absence of the analyte of interest. The test results may be visualized directly, or may measured using a reader (such as a scanner). The reader device may detect color or fluorescence from the readout area (for example, the test line and/or control line).

In another embodiment of a lateral flow device, there may be a second (or third, fourth, or more) test line located parallel or perpendicular (or in any other spatial relationship) to test line in test result zone. The operation of this particular embodiment is similar to that described in the immediately preceding paragraph with the additional considerations that (i) a second detector reagent specific for a second analyte, such as another antibody, may also be contained in the conjugate pad, and (ii) the second test line will contain a second specific binding partner having affinity for a second analyte, such as a second protein in the sample. Similarly, if a third (or more) test line is included, the test line will contain a third (or more) specific binding partner having affinity for a third (or more) analyte.

### 1. Sample Pad

The sample pad is a component of a lateral flow device that initially receives the sample, and may serve to remove particulates from the sample. Among the various materials that may be used to construct a sample pad (such as glass fiber, woven fibers, screen, non-woven fibers, cellosic fibers or paper), a cellulose sample pad may be beneficial if a large bed volume (e.g., 250 μl/cm2) is a factor in a particular application. Sample pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%).

### 2. Membrane and Application Solution

The types of membranes useful in a lateral flow device (such as nitrocellulose (including pure nitrocellulose and modified nitrocellulose), nitrocellulose direct cast on polyester support, polyvinylidene fluoride, or nylon), and considerations for applying a capture reagent to such membranes have been discussed previously.

In some embodiments, membranes comprising nitrocellulose are preferably in the form of sheets or strips. The thickness of such sheets or strips may vary within wide limits, for example, from about 0.01 to 0.5 mm, from about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

### 3. Conjugate Pad

The conjugate pad serves to, among other things, hold a detector reagent. Suitable materials for the conjugate pad include glass fiber, polyester, paper, or surface modified polypropylene. In some embodiments, a detector reagent may be applied externally, for example, from a developer bottle, in which case a lateral flow device need not contain a conjugate pad (see, for example, U.S. Pat. No. 4,740,468).

Detector reagent(s) contained in a conjugate pad is typically released into solution upon application of the test sample. A conjugate pad may be treated with various substances to influence release of the detector reagent into solution. For example, the conjugate pad may be treated with PVA or PVP (0.5% to 2%) and/or Triton X-100 (0.5%). Other release agents include, without limitation, hydroxypropylmethyl cellulose, SDS, Brij and β-lactose. A mixture of two or more release agents may be used in any given application. In a particular disclosed embodiment, the detector reagent in conjugate pad is a gold-conjugated antibody.

### 4. Absorbent Pad

The use of an absorbent pad in a lateral flow device is optional. The absorbent pad acts to increase the total volume of sample that enters the device. This increased volume can be useful, for example, to wash away unbound analyte from the membrane. Any of a variety of materials is useful to prepare an absorbent pad, for example, cellulosic filters or paper. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad.

The following examples are provided to illustrate certain particular features and/or embodiments. These examples should not be construed to limit the disclosure to the particular features or embodiments described.

### EXAMPLES

### Example 1

### Protein Variants as Indicators of Frontotemporal Dementia

### Materials and Methods

### Human Samples

Sera samples from post-mortem pathologically confirmed FTD-TDP and FTD-Tau cases were obtained. All enrolled subjects signed an Institutional Review Board-approved informed allowing both clinical assessments during life and several options for brain and/or bodily organ donation after death. There were 12 FTD-TDP and 12 FTD-Tau cases. 8 ante-mortem controls were also obtained. These controls have been previously screened for reactivity with a panel of scFvs and little to no reactivity was detected. The patient characteristics of the cohort are included in Table 1 (see FIG. 7).

### ScFvs

ScFvs reactive with multiple variants of TDP-43, beta-amyloid, tau and alpha-synuclein were previously isolated using our AFM based biopanning process. In the current study, a panel of 14 was used to capture scFvs to screen the sera samples. Seven scFvs that are reactive with variants of TDP-43 including AD-TDP1, AD-TDP2, AD-TDP3, ALS-TDP10, ALS-TDP14, SFTD-TDP1 and SFTD-TDP2, three scFvs that are reactive with oligomeric variants of beta-amyloid including A4, E1 and C6T, two scFvs that are reactive with oligomeric variants of tau including F9T and D11C and two scFvs that are reactive with oligomeric variants of alpha-synuclein including 10H and D5 were included in the panel. To produce these scFvs we utilized our previously published protocol ((Barkhordarian, Emadi, Schulz, & Sierks, 2006; Emadi, Barkhordarian, Wang, Schulz, & Sierks, 2007; Emadi, Kasturirangan, Wang, Schulz, & Sierks, 2009; Emadi et al., 2004; Kasturirangan et al., 2012; Kasturirangan et al., 2013; Liu et al., 2004; Marcus, Wang, Lindsay, & Sierks, 2008; H Tian et al., 2014; S. M. Williams et al., 2015; Zameer, Kasturirangan, Emadi, Nimmagadda, & Sierks, 2008; Zameer, Schulz, Wang, & Sierks, 2006; Zhou, Emadi, Sierks, & Messer, 2004, each of which is hereby incorporated by reference in its entirety).

### Detection Phages

The detection antibodies used in our capture ELISAs are biotinylated phage particles encompassing a scFv that is reactive with normal and abnormal variants of a particular protein. TDPM1 was used for detection of TDP-43 variants, H1V2 for Aβ variants, TauM1 for tau variants and D10 for α-synuclein variants (Emadi et al., 2004; H. Tian et al., 2015; S. M. Williams, Khan, Harris, Ravits, & Sierks, 2017; Yuan, Schulz, & Sierks, 2006, each of which is hereby incorporated by reference). The phage particle production and biotinylation protocols have been previously described (S. Williams, Schulz, & Sierks, 2015 which is hereby incorporated by reference).

### Phage Capture ELISA

To screen the sera samples for their protein variants composition, a sensitive phage capture ELISA system was utilized which has been previously described in detail (Spencer et al., 2016; S. M. Williams, Khan, et al., 2017; S. M. Williams, Schulz, Rosenberry, Caselli, & Sierks, 2017; S. M. Williams, Schulz, & Sierks, 2016; S. Williams et al., 2015; S. Williams et al., 2016, each of which is hereby incorporated by reference in its entirety).

### Statistical Analysis

The reactivity of each sample with the different scFvs was represented as relative standard deviation (SD). To calculate the relative SD, the average raw absorbance for the control samples was subtracted from each individual sample and the resulting value divided by the standard deviation of the controls. Cut-off values were designated to ensure no selection of control cases and only test samples above these values were considered positive. The figures and statistical analyses were completed using the IBM SPSS Statistics 24 program. Statistical significance is based on independent samples T-tests at p<0.05. ROC analysis and one-tailed bivariate correlations at p<0.05 were also completed using this program.

### Results

### Protein Variants Content

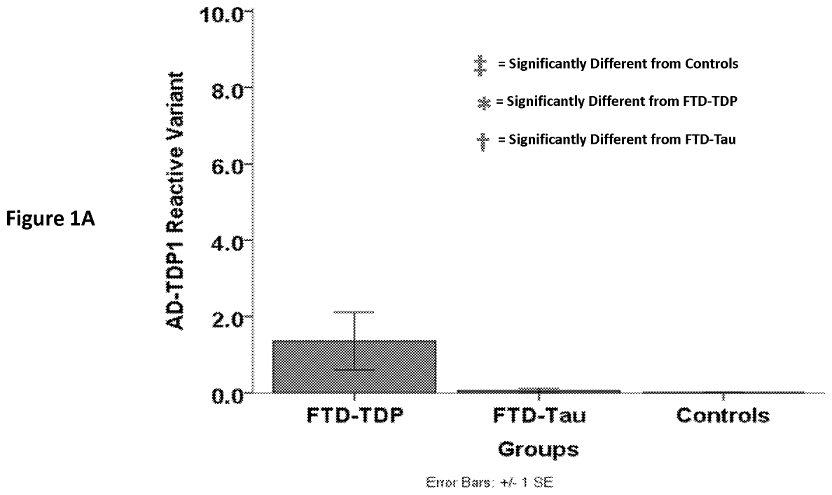

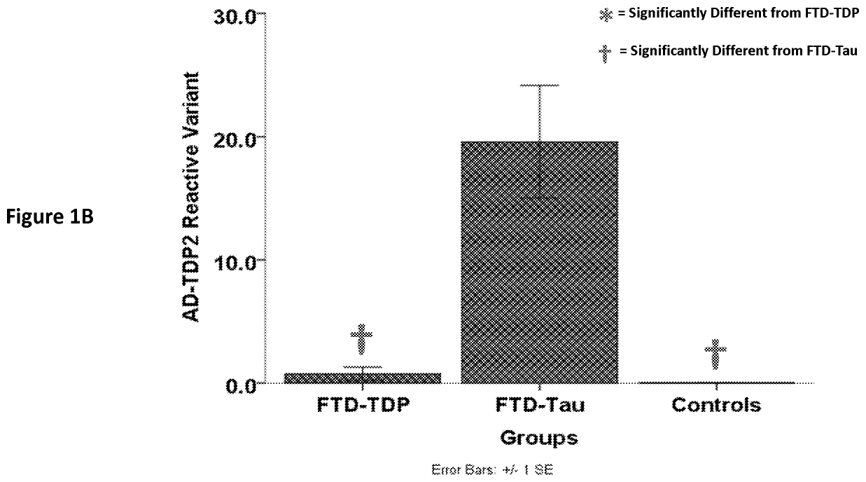

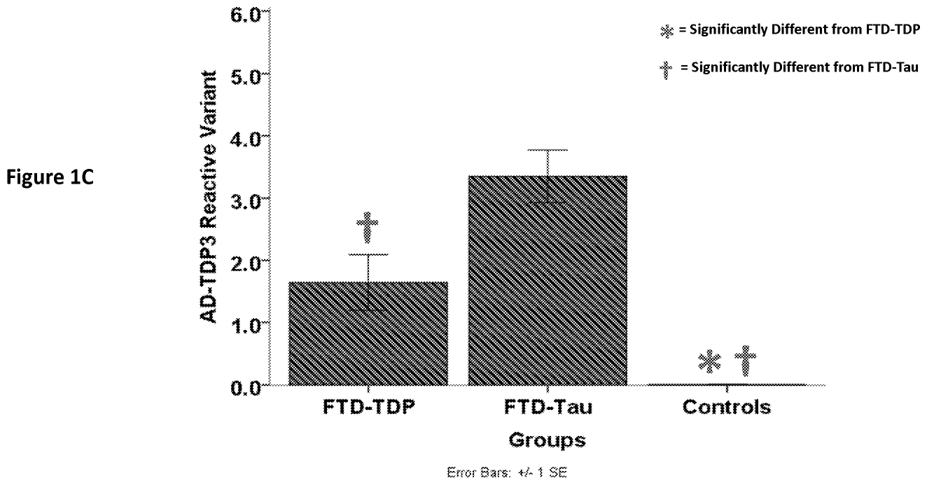

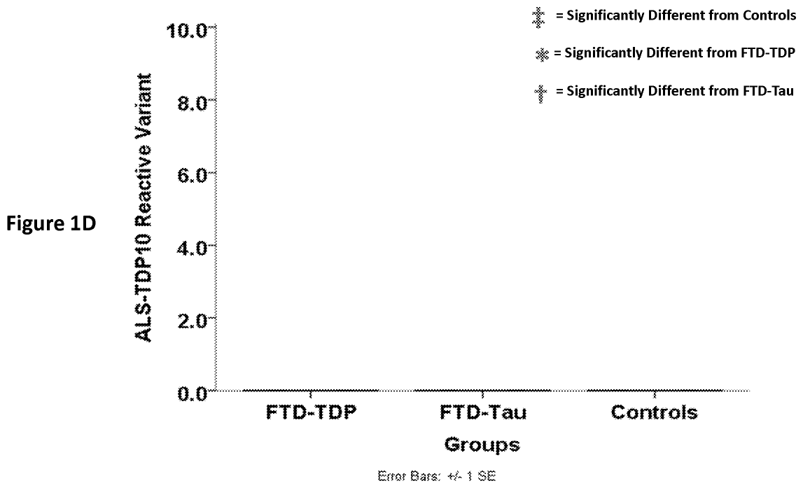

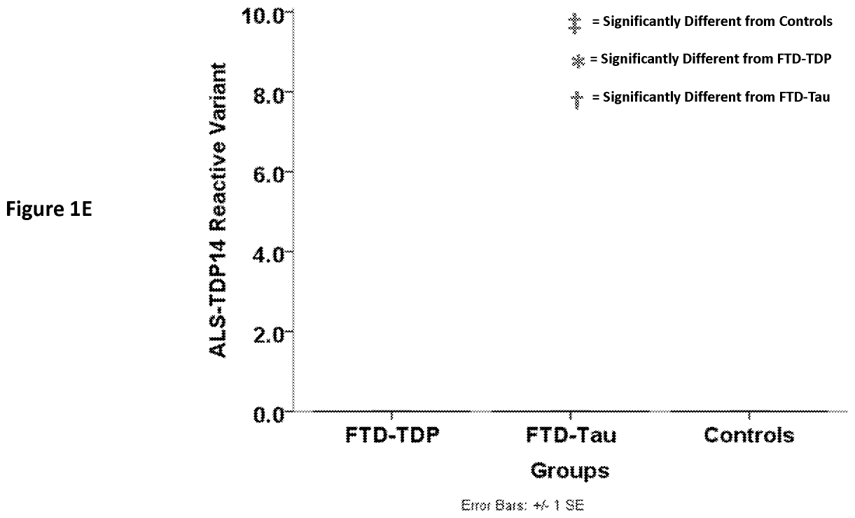

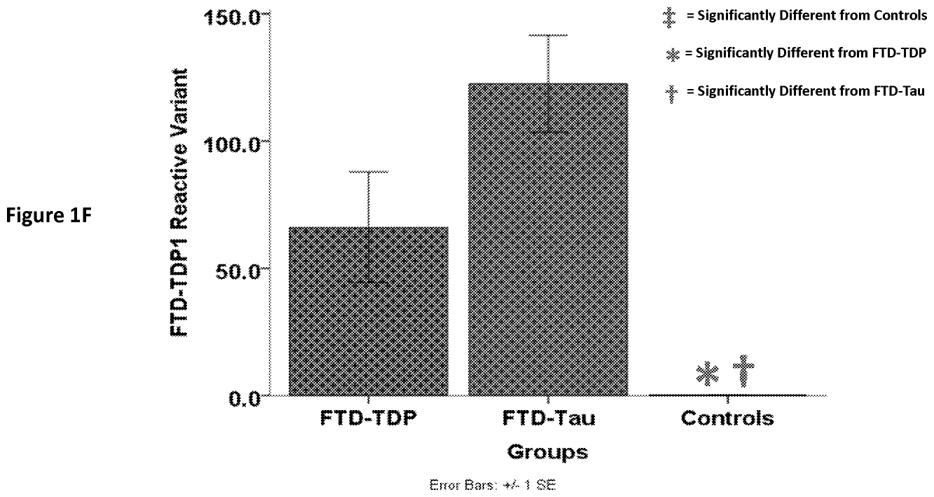

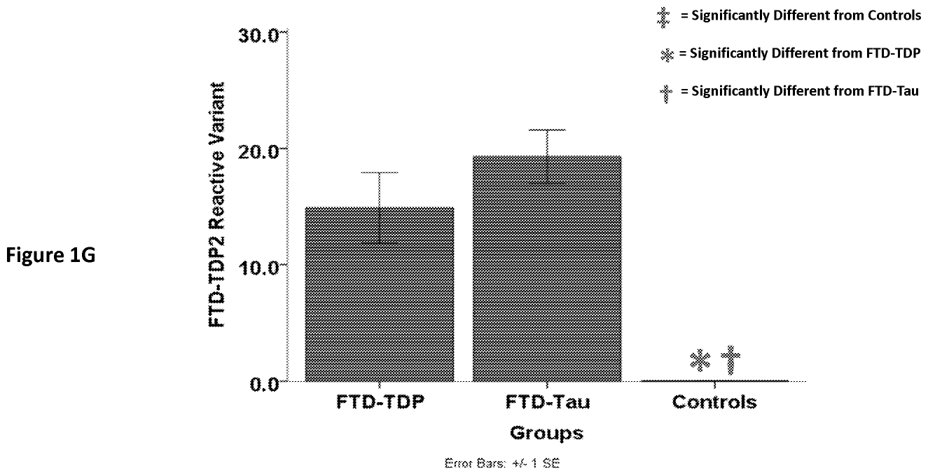

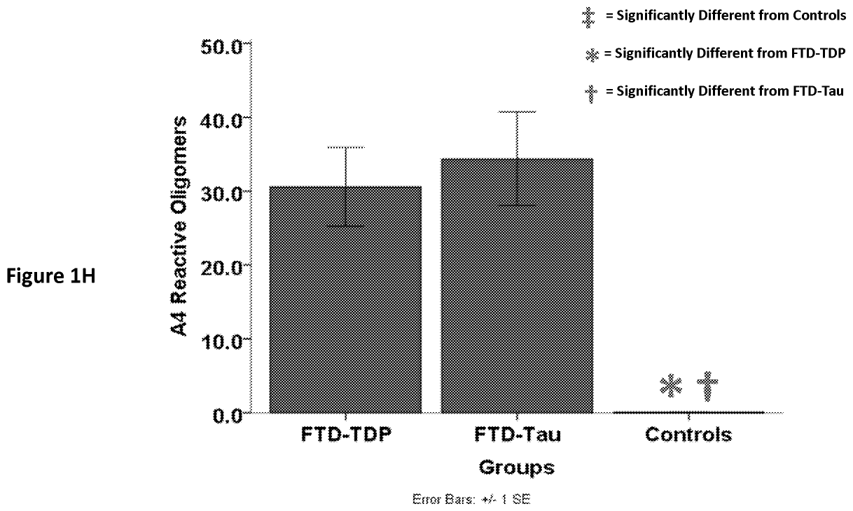

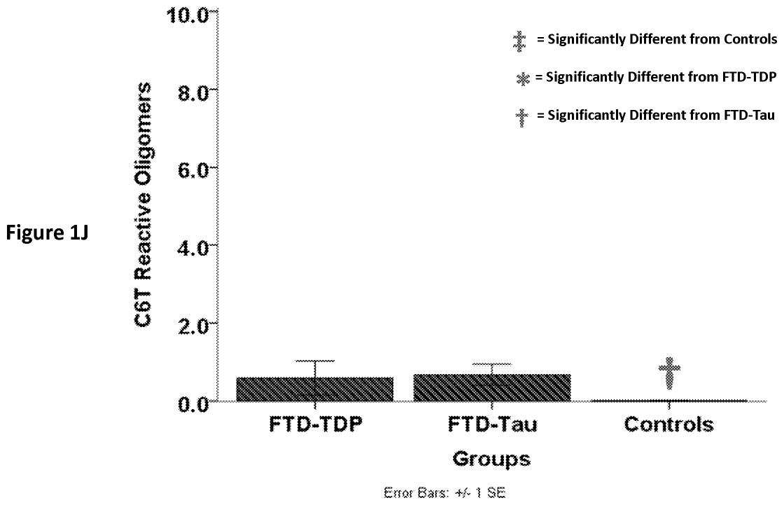

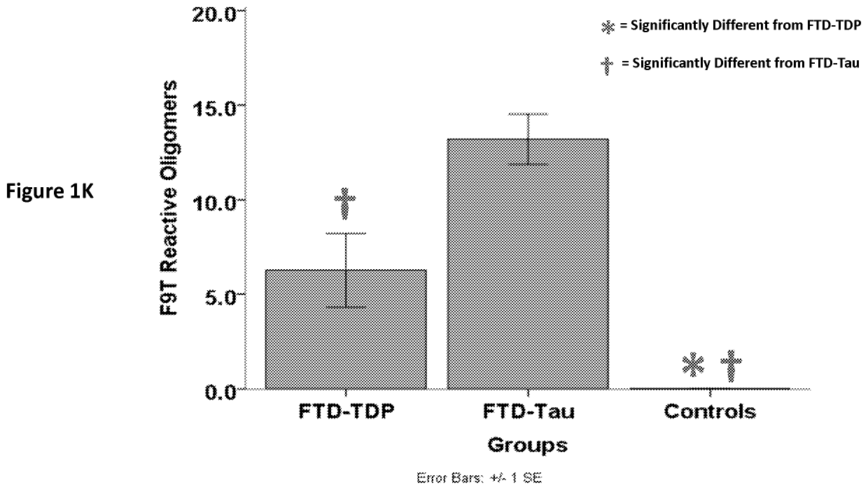

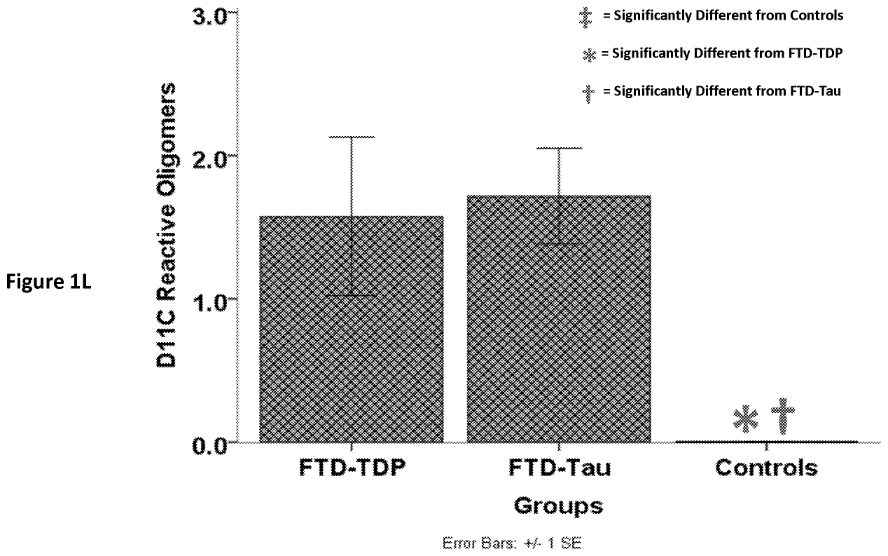

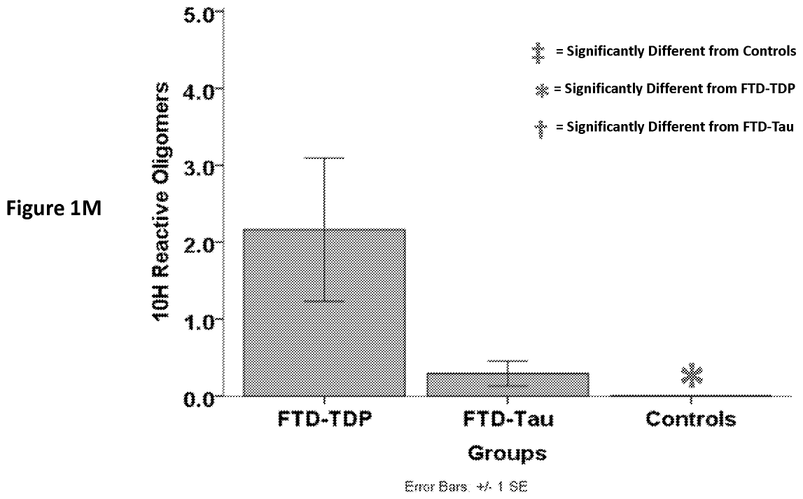

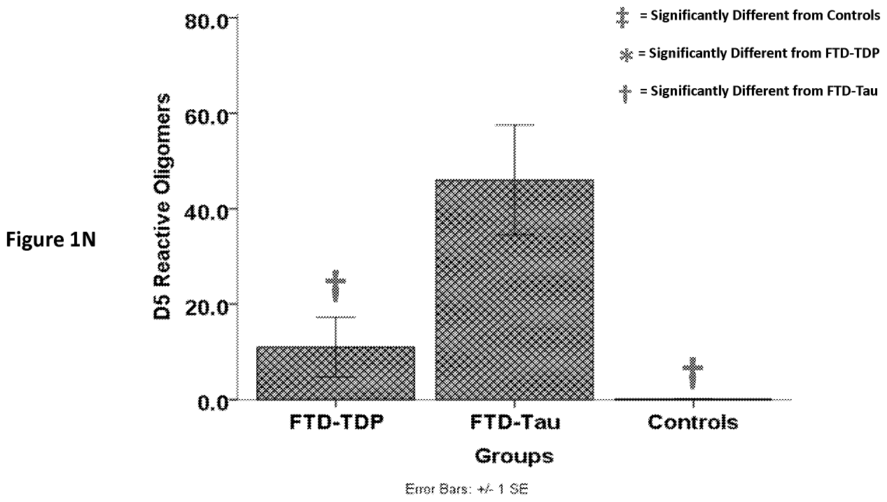

Following ELISA analysis of the sera cases with the TDP-43 reactive scFvs, little to no AD-TDP1 reactive variant was present in all three sample groups (FIG. 1A). However, AD-TDP2 reactive variant was significantly elevated mainly in the FTD-Tau cases, while the AD-TDP3 reactive variant was significantly heightened in both the FTD-TDP and FTD-Tau cases compared to the controls with average levels also being significantly elevated in the FTD-Tau cases compared to the FTD-TDP cases (FIGS. 1B, C). No ALS-TDP10 and ALS-TDP14 reactive variants were detected in the different groups (FIGS. 1D, E). FTD-TDP1 and FTD-TDP2 reactive variants were significantly elevated in both the FTD-TDP and FTD-Tau groups compared to the controls (FIGS. 1F, G). The scFvs A4 and E1, which are reactive with oligomeric variants of beta-amyloid, produced statistically significant reactivity with both the FTD-TDP and FTD-Tau cases compared to the controls (FIGS. 1H, I). The third oligomeric beta-amyloid reactive scFv C6T generated little reactivity with the cases, although there was a significant difference between the FTD-Tau and control groups (FIG. 1J). F9T and D11C, the scFvs that recognize different oligomeric variants of tau, were significantly more reactive with the FTD-TDP and FTD-Tau cases compared to the controls with F9T being significantly more reactive with the FTD-Tau cases compared to the FTD-TDP cases (FIGS. 1K, L). Lastly, using the oligomeric alpha-synuclein reactive scFvs 10H and D5, 10H was significantly more reactive with the FTD-TDP cases compared to the controls while D5 was significantly more reactive with the FTD-Tau cases compared to both the FTD-TDP and control groups (FIGS. 1M, N). Significant differences are based on independent sample t tests at p<0.05. FIG. 10 provides a bar graph illustrating cumulative protein variants in control and sera samples.

### Diagnostic Proficiency

In FIGS. 2G and 2H, the reactivity of each scFv with the individual cases is illustrated. From this, it is evident that the patterns of reactivity are different between the FTD-TDP and FTD-Tau groups as well as amongst the samples within each of the groups, further supporting our view of personalized diagnostics. Based on ROC analysis, FTD-TDP2, A4 and E1 seemed to be excellent biomarkers of FTD-TDP pathology, while AD-TDP2, AD-TDP3, FTD-TDP1, FTD-TDP2, A4, E1, F9T, D11C and D5 seemed to be excellent indicators of FTD-Tau pathology (FIG. 8, Table 2). This is based on having a ROC above 0.9 and sensitivity and specificity above 90%.

### Relationship Between Protein Variants Content and Medical History

Using the cases with available Min-Mental State Examination (MMSE) scores, we completed one-tailed bivariate correlations between these scores and protein variants content. Increasing FTD-TDP2 (r=−0.703; p=0.001), E1 (r=−0.459; p=0.028) and 10H (r=−0.897; p=0.000) reactive variants correlated significantly with decreasing MMSE scores in the FTD-TDP cases (FIGS. 2A-C). In the FTD-Tau cases increasing A4 (r=−0.460; p=0.037), E1 (r=−0.512; p=0.021) and almost F9T (r=−0.406; p=0.059) reactive variants correlated significantly with decreasing MMSE scores (FIGS. 2D-F). Interestingly, the correlation coefficients and p-values generated with the FTD-Tau cases were inferior to those produced using the FTD-TDP cases. These results may be attributed to the fact that fewer of the FTD-Tau cases had low MMSE scores like those of the FTD-TDP cases. This is also evident in FIG. 7 (Table 1) where based on independent sample t test the p-value between the FTD-Tau cases and the controls was 0.047, almost approaching our p<0.05 cut-off.

Also available for the FTD-Tau cases was their Unified Parkinson's Disease Rating Scale (UPDRS) scores. Bivariate correlations between these scores and protein variants content revealed multiple highly significant positive correlations (FIGS. 3A-I). As the levels of AD-TDP2 (r=0.560; p=0.008), AD-TDP3 (r=0.747; p=0.000), FTD-TDP1 (r=0.770; p=0.000), FTD-TDP2 (r=0.768; p=0.000), A4 (r=0.525; p=0.013), E1 (r=0.742; p=0.000), F9T (r=0.637; p=0.002), D11C (r=0.630; p=0.003) and D5 (r=0.593; p=0.005) reactive variants increased so did the UPDRS scores. Interestingly, FTD-TDP2, which based on ROC analysis is an excellent biomarker of FTD-TDP and FTD-Tau pathology did not correlate significantly with MMSE scores from the FTD-Tau cases but correlated excellently with their UPDRS scores, while with the FTD-TDP cases FTD-TDP2 produced an excellent correlation with their MMSE scores (FIGS. 2A, 3D). This may be due to the FTD-Tau cases revealing their impairments in the UPDRS test rather than their MMSE scores. Similarly, E1 did produce a good correlation with the MMSE scores from the FTD-Tau cases but was excellent with their UPDRS scores (FIGS. 2E, 3F). Likewise, F9T reactive variants produced an almost significant negative correlation with MMSE score in the FTD-Tau cases but correlated excellently with their UPDRS scores (FIGS. 2F, 3G). Actually, both F9T and D11C reactive tau oligomers generated excellent correlations with increasing UPDRS scores suggesting that tau may play a role in the impairments indicated by this test in these FTD-Tau cases (FIGS. 3G, H).

### Relationship Between Protein Variants Content and Pathology

The relationship between the levels of the different protein variants and pathological findings including brain weight, Braak stage, plaque levels and tangle levels were determined. In the FTD-TDP cases there was a significant negative correlation between decreasing brain weight and increasing 10H reactive oligomers (r=−0.737; p=0.003) and an almost significant negative correlation between decreasing brain weight and FTD-TDP2 reactive variants (r=-0.463; p=0.065). In the FTD-Tau cases there was a significant negative correlation between decreasing brain weight and increasing E1 reactive oligomers (r=-0.499; p=0.049) and an almost significant negative correlation between decreasing brain weight and D11C reactive oligomers (r=−0.454; p=0.069). These results suggest that there may be an association between changing brain weight and the presence of neurodegenerative disease associated protein variants.

Of the 12 FTD-TDP cases, only 4 were classified as being Braak stage II or above, while all 12 FTD-Tau cases were as at least Braak stage II (FIG. 2G). Therefore, by combining the FTD-TDP and FTD-Tau cases how protein variants content changed from Braak stage 0 through Braak stage V was illustrated. For the most part, the cumulative levels of all the protein variants increased with increasing Braak stages, i.e. advancing disease pathology (FIG. 2H).

When examining total plaques and tangles levels, it seemed that only 4 of the FTD-TDP and 4 of the FTD-Tau cases had high levels of plaques (FIG. 3J). Similarly, most of the FTD-TDP cases did not have high total tangle levels, however, most of the FTD-Tau cases did have high total tangle levels (FIG. 3K). These increasing tangle levels in the FTD-tau cases produced bivariate correlations with several of the protein variants (FIG. 5). The total tangle density score was determined using the frontal, temporal and parietal lobes, hippocampal CA1 region and entorhinal/transentorhinal regions. There was a significant negative correlation between decreasing FTD-TDP2 variants levels and increasing tau tangles in the temporal lobe and almost significant correlations between increasing tau tangles and decreasing E1, cumulative beta-amyloid oligomers and cumulative tau oligomers levels (FIGS. 5A-D). In the hippocampal CA1 region, decreasing E1 reactive oligomers correlated significantly with increasing tau tangles, while the correlation with FTD-TDP2 was almost significant (FIGS. 5E-F). In the entorhinal/transentorhinal region, decreasing levels of both E1 and D11C reactive oligomers correlated negatively with increasing tau tangle levels (FIGS. 5G-H). When combining the levels of tau tangles in all 5 brain regions, there was a significant correlation with decreasing E1 reactive oligomers and an almost significant correlation with decreasing FTD-TDP2 reactive variants (FIGS. 5I-J). These results suggest that there may be relationship between the decline of these protein variants and the formation of end-stage tangles.

Relationship Between Protein Variants Content and Patient Characteristics Separation of the FTD-TDP and FTD-Tau cases based on APOE genotype indicated that there were no statistically significant differences in cumulative TDP-43, beta-amyloid, tau and alpha-synuclein variants amongst those with APOE 23, 33 or 34 genotypes (FIGS. 6A, B). This is particularly interesting since cumulative beta-amyloid oligomer levels are influenced by the presence of the APOE 4 allele in Alzheimer's disease. Although there was a slight increase in total protein variants content according the pattern APOE23<APOE33<APOE34, the differences were not significant. Similarly, there was no significant difference in cumulative TDP-43, beta-amyloid, tau and alpha-synuclein variants in males compared to females in the FTD-TDP-43 cases (FIG. 6C). There were almost significant increases in cumulative tau and cumulative protein variants levels in the female FTD-Tau cases compared to the males and a significant increase in cumulative TDP-43 protein variants mostly attributed to FTD-TDP1 reactive variants (FIG. 6D).

### References (Each of which is Hereby Incorporated by Reference in its Entirety)

Barkhordarian, H., Emadi, S., Schulz, P., & Sierks, M. R. (2006). Isolating recombinant antibodies against specific protein morphologies using atomic force microscopy and phage display technologies. Protein Eng Des Sel, 19 (11), 497-502.
Beach, T. G., Adler, C. H., Sue, L. I., Serrano, G., Shill, H. A., Walker, D. G., Sabbagh, M. N. (2015). Arizona Study of Aging and Neurodegenerative Disorders and Brain and Body Donation Program. Neuropathology, 35 (4), 354-389. doi: 10.1111/neup.12189.
Beach, T. G., Sue, L. I., Walker, D. G., Roher, A. E., Lue, L., Vedders, L., . . . . Rogers, J. (2008). The Sun Health Research Institute Brain Donation Program: description and experience, 1987-2007. Cell Tissue Bank, 9 (3), 229-245.
Emadi, S., Barkhordarian, H., Wang, M. S., Schulz, P., & Sierks, M. R. (2007). Isolation of a human single chain antibody fragment against oligomeric alpha-synuclein that inhibits aggregation and prevents alpha-synuclein-induced toxicity. J Mol Biol, 368 (4), 1132-1144.
Emadi, S., Kasturirangan, S., Wang, M. S., Schulz, P., & Sierks, M. R. (2009). Detecting morphologically distinct oligomeric forms of alpha-synuclein. J Biol Chem, 284 (17), 11048-11058.
Emadi, S., Liu, R., Yuan, B., Schulz, P., McAllister, C., Lyubchenko, Y., . . . . Sierks, M. R. (2004). Inhibiting Aggregation of alpha-Synuclein with Human Single Chain Antibody Fragments. Biochemistry, 43 (10), 2871-2878.
Kasturirangan, S., Li, L., Emadi, S., Boddapati, S., Schulz, P., & Sierks, M. R. (2012). Nanobody specific for oligomeric beta-amyloid stabilizes nontoxic form. Neurobiol Aging, 33 (7), 1320-1328. doi: S0197-4580 (10) 00400-8 [pii]10.1016/j.neurobiolaging. 2010.09.020.
Kasturirangan, S., Reasoner, T., Boddapati, S., Emadi, S., Valla, J., & Sierks, M. R. (2013). Isolation and Characterization of a Nanobody that Selectively Binds Brain Derived Oligomeric Beta-Amyloid Biotechnol Prog.
Liu, R., Yuan, B., Emadi, S., Zameer, A., Schulz, P., McAllister, C., . . . . Sierks, M. R. (2004). Single chain variable fragments against beta-amyloid (Abeta) can inhibit Abeta aggregation and prevent abeta-induced neurotoxicity. Biochemistry, 43 (22), 6959-6967.
Marcus, W. D., Wang, H., Lindsay, S. M., & Sierks, M. R. (2008). Characterization of an antibody scFv that recognizes fibrillar insulin and beta-amyloid using atomic force microscopy. Nanomedicine, 4 (1), 1-7.
Spencer, B., Williams, S., Rockenstein, E., Valera, E., Xin, W., Mante, M., . . . . Sierks, M. R. (2016). alpha-synuclein conformational antibodies fused to penetratin are effective in models of Lewy body disease. Ann Clin Transl Neurol, 3 (8), 588-606. doi: 10.1002/acn3.321
Tian, H, Davidowitz, E, Lopez, P., He, P., Schulz, P., Moe, J., & Sierks, MR. (2014). Isolation and characterization of antibody fragments selective for toxic oligomeric tau. Neurobiol Aging.
Tian, H., Davidowitz, E., Lopez, P., He, P., Schulz, P., Moe, J., & Sierks, M. R. (2015). Isolation and characterization of antibody fragments selective for toxic oligomeric tau. Neurobiol Aging, 36 (3), 1342-1355. doi: 10.1016/j.neurobiolaging.2014.12.002.
Williams, S. M., Khan, G., Harris, B. T., Ravits, J., & Sierks, M. R. (2017). TDP-43 protein variants as biomarkers in amyotrophic lateral sclerosis. BMC Neurosci, 18 (1), 20. doi: 10.1186/s12868-017-0334-7.
Williams, S. M., Schulz, P., Rosenberry, T. L., Caselli, R. J., & Sierks, M. R. (2017). Blood-Based Oligomeric and Other Protein Variant Biomarkers to Facilitate Pre-Symptomatic Diagnosis and Staging of Alzheimer's Disease. J Alzheimers Dis. doi: 10.3233/jad-161116.
Williams, S. M., Schulz, P., & Sierks, M. R. (2016). Oligomeric alpha-synuclein and beta-amyloid variants as potential biomarkers for Parkinson's and Alzheimer's diseases. Eur J Neurosci, 43 (1), 3-16. doi: 10.1111/ejn.13056.
Williams, S., Schulz, P., & Sierks, M. R. (2015). A sensitive phage-based capture ELISA for sub-femtomolar detection of protein variants directly from biological samples. Biotechnol Prog, 31 (1), 289-298. doi: 10.1002/btpr.1987.
Williams, S M., Venkataraman, L., Tian, H., Khan, G., Harris, B T, & Sierks, M R. (2016). Novel Atomic Force Microscopy Based Biopanning for Isolation of Morphology Specific Reagents against TDP-43 Variants in Amyotrophic Lateral Sclerosis JOVE.
Williams, Stephanie M., Venkataraman, Lalitha, Tian, Huilai, Khan, Galam, Harris, Brent T., & Sierks, Michael R. (2015). Novel Atomic Force Microscopy Based Biopanning for Isolation of Morphology Specific Reagents against TDP-43 Variants in Amyotrophic Lateral Sclerosis. (96), e52584. doi: doi: 10.3791/52584.
Yuan, B., Schulz, P., & Sierks, M. R. (2006). Improved Affinity Selection of an scFv Antibody Fragment Against-amyloid Using Phage Display Technology and Off-rate Based Selection. Electronic Journal of Biotechnology, 9 (2), 171-175.
Zameer, A., Kasturirangan, S., Emadi, S., Nimmagadda, S. V., & Sierks, M. R. (2008). Anti-oligomeric Abeta single-chain variable domain antibody blocks Abeta-induced toxicity against human neuroblastoma cells. J Mol Biol, 384 (4), 917-928.
Zameer, A., Schulz, P., Wang, M. S., & Sierks, M. R. (2006). Single Chain Fv Antibodies against the 25-35 Abeta Fragment Inhibit Aggregation and Toxicity of Abeta42. Biochemistry, 45 (38), 11532-11539.
Zhou, C., Emadi, S., Sierks, M. R., & Messer, A. (2004). A human single-chain Fv intrabody blocks aberrant cellular effects of overexpressed alpha-synuclein. Mol Ther, 10 (6), 1023-1031.

### Example 2

### Isolation and Characterization of Antibody Fragments Selective for Human FTD Brain Derived TDP-43 Variants

In this Example, a new atomic force microscopy (AFM) based biopanning protocol was use to isolate antibody fragments (scFvs) from a phage display library that selectively bind TDP variants present in human FTD but not cognitively normal age matched brain tissue. The scFvs (FTD-TDP1 through 5) were used to probe postmortem brain tissue and sera samples for the presence of FTD related TDP variants. The scFvs readily selected the FTD tissue and sera samples over age matched controls. The scFvs were used in immunohistochemical analysis of FTD and control brain slices where the reagents showed strong staining with TDP in FTD brain tissue slice. FTD-TDP1, FTD-TDP2, FTD-TDP4 and FTD-TDP5 all protected neuronal cells against FTD TDP induced toxicity suggesting potential therapeutic value.

These studies show existence of different disease specific TDP variants in FTD individuals. A panel of scFvs capable of recognizing these disease specific TDP variants in postmortem FTD tissue and sera samples over age matched controls have been identified and can thus serve as biomarker tools and possible therapeutic targets.

Although there is a familial component to FTD with mutations identified in MAPT, C9orf72 and GRN, extensive TDP-43 pathology has been observed in both familial and sporadic cases of FTD. TDP-43 is a TAR DNA binding nuclear protein, 414 amino acids in length coded by the TARDBP gene. TDP-43 is a molecular pathology in the FTD-ALS spectrum and is observed in more than 50% of FTD cases. It plays a key role in transcription and translation processes and is involved in alternate splicing, mRNA transport and serves as a shuttle between the nucleus and cytoplasm. In FTD, TDP-43 is translocated to the cytoplasm and the location and type of aggregates present differ in clinical subtypes of FTD. Elevated levels of TDP 43 are found in circulating CSF of FTD and ALS patients. Although the pathogenic mechanisms is not known, several studies indicate that TDP-43 can spread in a prion like fashion from neuron to neuron through the axonal pathway. TDP-43 is also implicated in ALS, where different strains of TDP-43 have been shown to spread at different rates in in vitro models, indicating presence of multiple toxic TDP variants. Different TDP-43 conformations with different levels of toxicity resulting in different pathologies (TDP type A-D) and disease phenotype have been identified. These TDP-43 variants exist due to post translational modifications such as hyperphosphorylation, polyubiquitination and truncation leading to C-terminal fragments that are toxic. Currently, there is a lack of accurate blood-based biomarkers for FTD irrespective of familial or sporadic origin. It was hypothesized that FTD specific TDP-43 variants can be used as unique biomarkers in early antemortem diagnosis distinguishing FTD from other neurodegenerative diseases. Here, a unique panel of scFvs capable of recognizing TDP variants that are present in human FTD patients was identified.

### Methods

Human specimens: Human brain tissue homogenates from motor cortex of FTD (n=3), ALS (n=3) and healthy controls (n=2) and immunoprecipitated TDP-43 from these pathologically validated cases were provided from Georgetown Brain Bank (Georgetown University Medical Center). These samples were used in the initial AFM based screening. Human postmortem brain tissue sections from the superior frontal cortex and sera samples from FTD and control were also obtained. The brain sections were used for immunohistochemistry studies and sera samples (FTD-TDP (n=12), FTD-Tau (n=12), AD (n=11)) used in ELISA characterization studies.

Panning using immunoprecipitated TDP-43: Frozen brain tissue samples were briefly homogenized. Briefly tissue was sonicated in cold lysis buffer: 25 mM HEPES NaOH (pH 7.9), 150 mM NaCl, 1.5 mM MgCl2, 0.2 mM EDTA, 0.5% Triton-X-100, 1 mM dithiothreitol, protease inhibitor cocktail. The homogenized sample was centrifuged, and the supernatant was frozen in −80° C. TDP-43 protein was immunoprecipitated from brain tissue homogenates which were pooled (3 FTD samples and 2 healthy controls) using a commercial polyclonal antibody against TDP-43 protein (ProteinTech Inc, Chicago, IL; Catalog #10782-2-AP). The immunoprecipitated samples were probed with 1:1000 dilution of commercially available anti-TDP antibody (ProteinTech Inc, Chicago, IL; Catalog #10782-2-AP) to verify the presence of TDP-43. A combination of commercially available phage display libraries with a variability of 108 and concentration of 1012 pfu/m were used for the panning. An AFM based selection process was used that uses exhaustive subtractive panning steps to remove non-specific phage binding clones as well as clones binding to off-target antigens including antibody fragments that bound to TDP-43 forms from healthy individuals and from ALS patients. Atomic force microscopy (AFM) imaging was performed after every subtractive panning step to ensure removal of all antibody fragments binding these off-target antigens. Phage that did not bind to any of the off-target antigens was used for the final positive selection round performed against TDP immunoprecipitated from pooled FTD brain tissue samples. For this positive panning step, the TDP IP preparation was deposited on mica since only nanogram quantities of the antigen are needed and the process can be monitored via AFM imaging. Phage were eluted using trypsin and TEA and grown on LB-Amp plates overnight at 37° C.

Phage and scFv purification: Phage obtained after the positive selection were sequenced to ensure that they encoded complete scFv sequences. After sequence validation, phage were amplified. Phage titers were performed to verify the concentration of phage (~109 pfu/mL). Soluble scFv were also prepared by transforming the plasmids from each phage into E. Coli strain HB2151. An overnight culture was used for growing scFv in 2xYT media at 37° C. for 3-4 hours. The scFvs were grown and purified using a protein A Sepharose affinity column (GE Healthcare). Molecular size of the scFvs was checked in both the supernatant and lysate fraction via western blot with 1:2000 dilution of anti-c-myc 9e10 primary antibody (SantaCruz; Catalog #sc-40) followed by 1:2000 dilution of secondary antibody goat anti-mouse HRP (SantaCruz; Catalog #sc-2005). The DNA sequences of the scFvs were also validated using MAFFT, a multiple sequence alignment software.

TDP phage biotinylation: An aliquot of the remaining phage pool that was recovered after exhaustive subtractive panning with BSA, and aggregated α-synuclein and TDP-43 immunoprecipitated from healthy control tissue was used to select a detection phage for sandwich ELISA. A phage expressing an scFv that binds to all forms of TDP-43 contained in both FTD and ALS samples was selected to increase signal to noise ratio in ELISA. This phage was biotinylated using the EZ-Link Pentylamine-Biotinylation kit (Thermo Scientific, USA). The detection phage binds TDP variants present in both FTD and ALS samples and does not compete for the same binding sites as the capture scFv in sandwich ELISA.

FTD tissue and sera analysis: Brain tissue homogenates from FTD (n=3), ALS (n=3) and healthy individuals (n=2) were pooled together and used for the initial screening assay. The pooled brain tissue homogenate was used to coat the plates and tested for reactivity with each of the phages. This assay was used to evaluate binding specificity of all the phage clones for FTD over ALS and cognitively normal control samples. Soluble antibody fragments (scFv) (FTD-TDP1, FTD-TDP2, FTD-TDP3, FTD-TDP4 and FTD2 TDP-5) were produced for each of the phages that had a high signal with the FTD brain tissue homogenates in the indirect ELISA. The scFvs were used as the capture antibody in a sandwich ELISA to test reactivity with sera samples (12 FTD-TDP, 12 FTD-tau and 10 healthy controls). The bound species were detected using biotinylated TDP phage to amplify the signal to noise ratio. Signal ratios were determined by comparing the signal obtained for each scFv with the FTD sera to healthy controls and plotted.

Western blot analysis: A 15% non-denaturing PAGE gel was used to analyze the molecular weight of TDP species recognized by the FTD-TDP2 scFv. The resolving and stacking gels were prepared without SDS. 5X-Running buffer (15 g Tris+72g Glycine in 1L) and 2X-loading buffer (62.5 mM Tris-HCl, pH 6.8, 25% glycerol, 1% Bromophenol Blue) were also prepared without SDS detergent. Protein samples were diluted in loading buffer and this mixture was loaded directly onto the gels without heat denaturation. Samples including two healthy control tissue samples, TDP-43 immunoprecipitated from two healthy controls and three different FTD individuals were analyzed. The gel apparatus was set at 70V for 30 minutes followed by 100V for approximately 3 hours until the marker was well separated. A nitrocellulose membrane was used to transfer the separated bands from the gel using standard western protocol. The blot was incubated at RT with 2% milk powder in 1× PBS followed by incubation with FTD-273 TDP2 scFv supernatant overnight at 4° C. The blot was then washed with 1× PBS thrice followed by incubation with anti-c-myc (9e10) primary antibody (1:2000 dilution) for 2 hours at RT. The blot was further washed with 1× PBS followed by incubation with goat anti-mouse HRP (1:1000 dilution) at RT for 45 minutes. After a final wash with 1×PBS, a colorimetric DAB substrate was added, and the blot was developed as per manufacturer's protocol.

Competition ELISA: To determine if the five FTD-TDP scFvs were binding to similar or different epitopes, a competition ELISA was performed. Each of the five FTD-TDP scFvs were pre-incubated with FTD sera at 37° C. for 1 hour. During the addition of antigen, 1:100 dilution of FTD sera or FTD sera pre-incubated with FTD-TDP scFvs were used.

Immunohistochemistry: Human postmortem tissue sections from superior frontal cortex were incubated with FTD-TDP2 and FTD-TDP3 scFvs respectively (1:100) on a shaking stage overnight at 4° C. Primary antibodies against c-myc region of scFv (Sigma, 1:1000, rabbit) and MAP2 (Covance, 1:400, mouse) were applied to the tissue sections for 3 hours at room temperature. Goat anti-rabbit IgG and goat anti-mouse IgG with fluorescence at the concentration of 1:1000 were used respectively as secondary antibodies for 1 hour at room temperature. The sections were washed with PBS 3 times and the non-specific background was blocked with 0.03% Sudan black for 5 minutes. The sections were observed and imaged with Leica SP5. Commercial MAP2 antibody is visualized in red, anti-TDP scFv in green and DAPI, which stains the nucleus.

Toxicity Assay: TDP-43 for the toxicity assay was immunoprecipitated from human postmortem FTD and control brain tissue using four commercial antibodies-A16583 (cell signaling), ab190963 (Abcam), 10782-2-AP and 12892-1-AP (ProteinTech). The human neuroblastoma cell line, SH-SY5Y was 6 well plate and once they reached confluence, toxicity was induced by incubating the cells with 1 μg/mL of TDP-43 IP from FTD or control. The cells were then incubated with commercial anti-TDP antibody ab 190963 (Abcam, 1 μg/mL), or one of the anti-TDP scFvs-FTD-TDP1, FTD-TDP2, FTD-TDP3, FTD-TDP4 and FTD-TDP5. After 12 hours of incubation, toxicity was measured using a lactate dehydrogenase assay kit.

Statistical Analysis: Luminescence signals obtained on the ELISAs were plotted as a ratio with respect to either background or healthy controls. Reactivity of each test sample was obtained relative to the average signal of the control group. Any sample with a ratio greater than 1 was considered a positive signal. Statistical significance was assessed using SPSS software (version 24) and one-way ANOVA with post-hoc analyses was performed with p<0.05. To determine the accuracy of the anti-TDP scFvs in detecting FTD over healthy controls, Receiver Operating Characteristic curves (ROC) and Area Under the Curve (AUC) were computed based on the reactivity of the five FTD-TDP scFvs with FTD-TDP, FTD-Tau and healthy control sera. Sensitivity and specificity of the FTD-TDP scFvs were also obtained by setting the cutoff as the average value of the healthy controls. 0.8 value for AUC is considered good while 0.5 (straight line) means it does not differentiate between FTD and control and is not a good diagnostic test.

Abbreviations FTD: Frontotemporal Dementia; TDP-43: TAR DNA Binding Protein 43; ALS: Amyotropic Lateral Sclerosis; scFv: single chain variable fragment; Aβ: Amyloid beta; AFM: Atomic Force Microscopy; BSA: bovine serum album; BCA: bicinchoninic acid; IPTG: isopropyl β-d-1-thiogalactopyranoside; IgG: Immunoglobulin G; ELISA: Enzyme Linked Immunosorbent Assay; EDTA: Ethylenediaminetetraacetic Acid; SDS-PAGE: Sodium Dodecyl Sulfate-Poly Acrylamide Gel Electrophoresis; DAPI: 4′,6-diamidino-2-phenylindole, One-way 322 ANOVA: One-way Analysis of Variance.

### Results

Phage and scFv purification: After serial rounds of subtractive panning were performed to remove phage that bound off target antigens including BSA, homogenate from healthy human brain tissue and TDP-43 immunoprecipitated from pooled ALS brain tissue homogenates, a single round of positive selection was performed using immunoprecipitated TDP-43 from pooled FTD brain tissue (FIGS. 9A-9C). Eighty phage clones were recovered from the positive panning step and were verified for sequence integrity by DNA sequencing. The reactivity of 17 phage clones were further tested to verify that they bound human FTD brain tissue, but not ALS or healthy brain tissue homogenates using pooled tissue homogenates. All 17 phage preparations showed high reactivity to the FTD sample with very little or no reactivity to the ALS sample (FIG. 10A). Based on the initial ELISA screening, we selected eight phage clones with the highest reactivity with pooled FTD but no reactivity with pooled ALS and pooled healthy control tissue homogenates for further testing with individual FTD (n=6) and age-matched cognitively normal (n=2) brain homogenates (FIG. 10B). The 8 phage samples reacted with each of the FTD brain tissue homogenates, however each phage had a different binding pattern among the FTD patients suggesting that they bind different TDP variants. The five phage clones that showed the strongest reactivity toward the individual FTD tissue samples and lowest reactivity towards the control samples were expressed as scFvs and used to determine if the TDP variants could also be detected in sera samples. The five scFvs (FTD-TDP1, FTD-TDP2, FTD-TDP3, FTD-TDP4 and FTD-TDP5) were used to assay sera samples from FTD111 TDP (n=12), FTD-tau (n=12), AD sera (n=11) and controls (n=10) (FIG. 11A-11E). Four of the scFvs (FTD112 TDP1, FTD-TDP2, FTD-TDP3 and FTD-TDP4) have significantly higher reactivity to FTD-TDP and FTD-tau sera samples compared to AD sera samples, while the fifth scFv (FTD-TDP5) had high reactivity with all the FTD and AD samples. None of the scFvs studied here discriminated between FTD-TDP and FTD-tau sera samples, though four of them did discriminate between FTD and AD samples suggesting that some TDP variants are unique to FTD, while others are involved in both FTD and AD. The sensitivity and specificity of each of the five anti-TDP scFvs for FTD TDP and FTD-tau are shown (Table 4). All the scFvs have area under curve (AUC)>0.84 implying high sensitivity and specificity of the scFvs in selecting FTD sera over healthy controls.

TABLE 4
Sensitivity and specificity of five anti-TDP FTD scFvs based on reactivity with FTD-TDP (n = 12),
FTD-tau (n = 12) and control sera (n = 8). Sensitivity and specificity were calculated for FTD-TDP,
FTD-tau and both FTD subtypes.

| | FTD-TDP | | | FTD-Tau | | | Total | | |
|---|---|---|---|---|---|---|---|---|---|
| scFv | Sensitivity | Specificity | AUC | Sensitivity | Specificity | AUC | Sensitivity | Specificity | AUC |
| FTD- | 91.66% | 100% | 0.99 | 75% | 75% | 0.85 | 75% | 87.5% | 0.92 |
| TDP1 | | | | | | | | | |
| FTD- | 75% | 100% | 0.87 | 75% | 100% | 0.82 | 75% | 100% | 0.84 |
| TDP2 | | | | | | | | | |
| FTD- | 100% | 87.5% | 0.99 | 91.66% | 87.5% | 0.96 | 95.83% | 100% | 0.99 |
| TDP3 | | | | | | | | | |
| FTD- | 100% | 83.3% | 0.97 | 75% | 75% | 0.79 | 75% | 87.5% | 0.88 |
| TDP4 | | | | | | | | | |
| FTD- | 100% | 100% | 1 | 100% | 100% | 1 | 100% | 100% | 1 |
| TDP5 | | | | | | | | | |

Western blot analysis: It was assumed that the FTD selective scFvs bind conformational epitopes of TDP-43 that are involved in FTD since the scFvs did not bind TDP variants present in healthy age-matched control tissue. To verify that the scFvs were binding a conformational epitope, PAGE gels were analyzed under denaturing (FIGS. 16A and 16B) and native conditions by probing with a commercial anti-TDP antibody and the FTD-TDP2 scFv (FIGS. 12A and 12B). Under native conditions, FTD-TDP2 scFv recognizes a disease variant of TDP-43 present in FTD (approximately 70 kDa), but not 129 in healthy control tissue or TDP-43 immunoprecipitated from healthy control tissue.

Competition ELISA: To determine if the five different scFvs against the FTD related TDP variants were binding different epitopes, a competition ELISA was performed where each scFv was tested with FTD sera (no competition) or FTD sera preincubated with one of the other 4 scFvs (competition) (FIG. 13). If any two scFvs recognize the same epitope, a significant reduction in ELISA signal was predicted. One-way ANOVA analysis indicated there was not any difference between the control samples and those with added scFv indicating that the scFvs bind unique epitopes.

Immunohistochemistry: Two anti-TDP scFvs were further studied using IHC analysis of human postmortem FTD and control brain tissue sections. The FTD-TDP2 and FTD-TDP3 scFvs were utilized for the IHC analyses since they had high expression levels and high reactivity with FTD over cognitively normal controls in tissue and sera analyses. Fluorescence tagged secondary antibodies were used to visualize the microtubule associated protein (MAP2) and the bound scFvs (FIGS. 15A and 15B). Although MAP2 staining is present in both FTD and control cases, there is no anti-TDP scFv staining with the control case. In the FTD case, there is extensive anti-TDP scFv staining indicating that the FTD-TDP scFvs recognize disease specific TDP variants. Both the anti-TDP scFvs have similar staining surrounding the nucleus in FTD tissue indicating presence of intraneuronal TDP variants in the cytoplasm.

Toxicity assay: When incubated with neuronal cells, the TDP-43 sample immunoprecipitated from human post mortem FTD brain tissue induced significantly increased toxicity toward cultured SH-SY5Y cells compared to TDP-43 immunoprecipitated from cognitively normal human brain tissue (FIGS. 16A and 16B).

### DISCUSSION

TDP-43 pathology is commonly observed in a vast number of FTD cases and TDP-43 variants are observed in CSF and sera making it an ideal candidate for antemortem FTD diagnosis. A panel of scFvs was generated that selectively bind FTD specific TDP-43 variants using an AFM-based biopanning protocol. Five scFvs that had high reactivity with individual FTD brain tissue over control tissue (FTD-TDP1, FTD-TDP2, FTD-TDP4 and FTD-TDP5) were further analyzed using sera samples from FTD-TDP, FTD-tau, AD, and control cases (FIGS. 9A-11E). Four of the five scFvs tested showed high reactivity with FTD sera but not AD or cognitively normal controls, while one scFv showed high reactivity with the FTD and AD sera cases (FIGS. 11A-11E). Even though around 50% of AD cases have prominent TDP pathology, it is apparent that TDP pathology in FTD cases has some distinct differences from that in AD cases. Although FTD sera has been classified as FTD-TDP and FTD-tau based on postmortem pathology reports, studies have shown that there is an overlap of tau and TDP-43 pathology in FTD cases. Here, it was also observed that TDP pathology between the FTD-TDP and FTD-tau cases are quite similar (FIGS. 10A-11E).

TDP-43 undergoes several post-translational modifications and occurs as 177 C-terminal fragments of varying lengths. Studies have indicated that a 70 kDa species is present in FTD brain tissue studies. Here, it has been shows that FTD-TDP2 scFv recognizes a conformation specific 70 kDa species present in FTD and not in cognitively normal healthy control tissue samples (FIGS. 12A and 12B) and that this variant is localized in the cytoplasm of neurons in FTD brain tissue, but not healthy controls (FIGS. 15A and 15B). Other neurodegenerative diseases like motor neuron disease, AD, dementia with Lewy bodies and Huntington's disease also exhibit TDP pathology. It was also shown that the TDP variants present in FTD brain are toxic to neuronal cells (FIGS. 16A and 16B), and that selectively targeting the toxic variants can be an effective therapeutic option for treating FTD and other TDP related diseases. A panel of scFvs was generated that selectively bind TDP-43 protein variants present in postmortem FTD brain tissue and sera samples but not age matched, healthy controls. These results indicate the diagnostic potential of these scFvs in distinguishing FTD from healthy controls and other TDP-43 pathologies.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of diagnosing a subject with frontotemporal dementia (FTD), the method comprising:
contacting a biological sample from a subject at risk or suspected of having FTD with a single-chain variable fragment (scFv) reactive with one or more oligomeric beta amyloid disease variants, wherein the scFv reactive with one or more oligomeric beta amyloid disease variants has a light chain complementary determining region (CDR) and heavy chain CDR combination selected from the group consisting of:
SEQ ID NOS: 21 (CDR-L1), 26 (CDR-L2), 43 (CDR-L3), 62 (CDR-H1), 66 (CDR-H2), and 85 (CDR-H3); and
SEQ ID NOS: 21 (CDR-L1), 27 (CDR-L2), 44 (CDR-L3), 61 (CDR-H1), 67 (CDR-H2), and 85 (CDR-H3); and
detecting binding between the scFvs and the biological sample, wherein the detection of binding between the scFv to the biological sample is indicative that the subject has or is likely to develop FTD.

2. The method of claim 1, wherein the scFv reactive with one or more oligomeric beta amyloid disease variants has the amino acid sequence set forth in SEQ ID NO: 3 or SEQ ID NO: 4.

3. The method of claim 1, further comprising:
contacting the biological sample from the subject at risk or suspected of having FTD with one or more additional scFvs reactive with TAR DNA-binding protein 43 (TDP-43) disease variants, alpha-syn disease variants, or tau disease variants, wherein the one or more additional scFvs reactive with TDP-43 disease variants, alpha-syn disease variants, or tau disease variants has a light chain CDR and heavy chain CDR combination or a light chain CDR combination and truncated heavy chain selected from the group consisting of:
SEQ ID NOS: 21 (CDR-L1), 24 (CDR-L2), 41 (CDR-L3), 61 (CDR-H1), 64 (CDR-H2), and 83 (CDR-H3);
SEQ ID NOS: 21 (CDR-L1), 24 (CDR-L2), 41 (CDR-L3), 101 (CDR-H1), 103 (CDR-H2), and 83 (CDR-H3);
SEQ ID NOS: 21 (CDR-L1), 25 (CDR-L2), 42 (CDR-L3), 61 (CDR-H1), 65 (CDR-H2), and 84 (CDR-H3);
SEQ ID NOS: 21 (CDR-L1), 28 (CDR-L2), 45 (CDR-L3), 61 (CDR-H1), 68 (CDR-H2), and 87 (CDR-H3);
SEQ ID NOS: 21 (CDR-L1), 28 (CDR-L2), 45 (CDR-L3), 101 (CDR-H1), 106 (CDR-H2), and 87 (CDR-H3);
SEQ ID NOS: 22 (CDR-L1), 29 (CDR-L2), 46 (CDR-L3), 61 (CDR-H1), 69 (CDR-H2), and 88 (CDR-H3);
SEQ ID NOS: 22 (CDR-L1), 29 (CDR-L2), 46 (CDR-L3), 101 (CDR-H1), 107 (CDR-H2), and 88 (CDR-H3);
SEQ ID NOS: 23 (CDR-L1), 30 (CDR-L2), 47 (CDR-L3), 63 (CDR-H1), 70 (CDR-H2), and 89 (CDR-H3);
SEQ ID NOS: 23 (CDR-L1), 30 (CDR-L2), 47 (CDR-L3), 102 (CDR-H1), 108 (CDR-H2), and 89 (CDR-H3);
SEQ ID NOS: 21 (CDR-L1), 32 (CDR-L2), and 49 (CDR-L3);
SEQ ID NOS: 21 (CDR-L1), 27 (CDR-L2), 50 (CDR-L3), 61 (CDR-H1), 72 (CDR-H2), and 91 (CDR-H3);
SEQ ID NOS: 21 (CDR-L1), 26 (CDR-L2), 51 (CDR-L3), 61 (CDR-H1), 73 (CDR-H2), and 92 (CDR-H3);
SEQ ID NOS: 21 (CDR-L1), 26 (CDR-L2), 51 (CDR-L3), 101 (CDR-H1), 110 (CDR-H2), and 92 (CDR-H3);
SEQ ID NOS: 21 (CDR-L1), 33 (CDR-L2), 52 (CDR-L3), 61 (CDR-H1), and 74 (CDR-H2);
SEQ ID NOS: 21 (CDR-L1), 33 (CDR-L2), 52 (CDR-L3), 101 (CDR-H1), and 111 (CDR-H2);
SEQ ID NOS: 21 (CDR-L1), 34 (CDR-L2), 53 (CDR-L3), 61 (CDR-H1), 75 (CDR-H2), and 93 (CDR-H3);
SEQ ID NOS: 21 (CDR-L1), 34 (CDR-L2), 53 (CDR-L3), 101 (CDR-H1), 112 (CDR-H2), and 93 (CDR-H3);
SEQ ID NOS: 21 (CDR-L1), 35 (CDR-L2), 54 (CDR-L3), 61 (CDR-H1), 76 (CDR-H2), and 94 (CDR-H3);
SEQ ID NOS: 21 (CDR-L1), 35 (CDR-L2), 54 (CDR-L3), 101 (CDR-H1), 113 (CDR-H2), and 94 (CDR-H3);
SEQ ID NOS: 21 (CDR-L1), 36 (CDR-L2), 55 (CDR-L3), 61 (CDR-H1), 77 (CDR-H2), and 95 (CDR-H3); and
SEQ ID NOS: 21 (CDR-L1), 36 (CDR-L2), 55 (CDR-L3), 101 (CDR-H1), 114 (CDR-H2), and 95 (CDR-H3); and
detecting binding between the one or more additional scFvs reactive with TAR DNA-binding protein 43 (TDP-43) disease variants, alpha-syn disease variants, or tau disease variants and the biological sample, wherein the detection of binding between the one or more additional scFvs to the biological sample is indicative that the subject has or is likely to develop FTD.

4. The method of claim 3, wherein the scFv reactive with one or more TDP-43 disease variants has the amino acid sequence set forth in one of SEQ ID NOs: 8-20.

5. The method of claim 3, wherein the one or more additional scFvs is reactive with one or more alpha-syn disease variants has the amino acid sequence set forth in SEQ ID NO: 1, SEQ ID NO: 2, or SEQ ID NO: 5.

6. The method of claim 3, wherein the one or more additional scFvs is reactive with one or more tau disease variants has the amino acid sequence set forth in SEQ ID NO: 6 or SEQ ID NO: 7.

7. The method of claim 1, wherein the biological sample is a serum sample.

8. The method of claim 1, wherein the method is performed utilizing an enzyme linked immunosorbent assay (ELISA).

9. The method of claim 1, further comprising administering a treatment to the subject diagnosed with FTD to treat one or more symptoms associated with FTD.

10. A method of monitoring a subject with frontotemporal dementia (FTD), the method comprising:
contacting a first biological sample from a subject diagnosed with FTD with a single-chain variable fragment (scFv) reactive with one or more oligomeric beta amyloid disease variants, wherein the scFv reactive with one or more oligomeric beta amyloid disease variants has a light chain complementary determining region (CDR) and heavy chain CDR combination selected from the group consisting of:
SEQ ID NOS: 21 (CDR-L1), 26 (CDR-L2), 43 (CDR-L3), 62 (CDR-H1), 66 (CDR-H2), and 85 (CDR-H3); and
SEQ ID NOS: 21 (CDR-L1), 27 (CDR-L2), 44 (CDR-L3), 61 (CDR-H1), 67 (CDR-H2), and 85 (CDR-H3);
detecting binding between the one or more scFvs and the first biological sample;
administering a treatment to the subject diagnosed with FTD;
at a desired time, contacting a second biological sample obtained from the subject with one or more scFvs reactive with one or more oligomeric beta amyloid disease variants; and
detecting binding between the one or more scFvs and the second biological sample.

11. The method of claim 10, wherein the scFv reactive with one or more oligomeric beta amyloid disease variants has the amino acid sequence set forth in SEQ ID NO: 3 or SEQ ID NO: 4.

12. The method of claim 10, further comprising:
contacting the first biological sample and/or the second biological sample from the subject diagnosed with FTD with one or more additional scFvs reactive with TAR DNA-binding protein 43 (TDP-43) disease variants, alpha-syn disease variants, or tau disease variants, wherein the one or more additional scFvs reactive with TDP-43 disease variants, alpha-syn disease variants, or tau disease variants has a light chain CDR and heavy chain CDR combination or a light chain CDR combination and truncated heavy chain selected from the group consisting of:
SEQ ID NOS: 21 (CDR-L1), 24 (CDR-L2), 41 (CDR-L3), 61 (CDR-H1), 64 (CDR-H2), and 83 (CDR-H3);
SEQ ID NOS: 21 (CDR-L1), 24 (CDR-L2), 41 (CDR-L3), 101 (CDR-H1), 103 (CDR-H2), and 83 (CDR-H3);
SEQ ID NOS: 21 (CDR-L1), 25 (CDR-L2), 42 (CDR-L3), 61 (CDR-H1), 65 (CDR-H2), and 84 (CDR-H3);
SEQ ID NOS: 21 (CDR-L1), 28 (CDR-L2), 45 (CDR-L3), 61 (CDR-H1), 68 (CDR-H2), and 87 (CDR-H3);
SEQ ID NOS: 21 (CDR-L1), 28 (CDR-L2), 45 (CDR-L3), 101 (CDR-H1), 106 (CDR-H2), and 87 (CDR-H3);
SEQ ID NOS: 22 (CDR-L1), 29 (CDR-L2), 46 (CDR-L3), 61 (CDR-H1), 69 (CDR-H2), and 88 (CDR-H3);
SEQ ID NOS: 22 (CDR-L1), 29 (CDR-L2), 46 (CDR-L3), 101 (CDR-H1), 107 (CDR-H2), and 88 (CDR-H3);
SEQ ID NOS: 23 (CDR-L1), 30 (CDR-L2), 47 (CDR-L3), 63 (CDR-H1), 70 (CDR-H2), and 89 (CDR-H3);
SEQ ID NOS: 23 (CDR-L1), 30 (CDR-L2), 47 (CDR-L3), 102 (CDR-H1), 108 (CDR-H2), and 89 (CDR-H3);
SEQ ID NOS: 21 (CDR-L1), 32 (CDR-L2), and 49 (CDR-L3);
SEQ ID NOS: 21 (CDR-L1), 27 (CDR-L2), 50 (CDR-L3), 61 (CDR-H1), 72 (CDR-H2), and 91 (CDR-H3);
SEQ ID NOS: 21 (CDR-L1), 26 (CDR-L2), 51 (CDR-L3), 61 (CDR-H1), 73 (CDR-H2), and 92 (CDR-H3);
SEQ ID NOS: 21 (CDR-L1), 26 (CDR-L2), 51 (CDR-L3), 101 (CDR-H1), 110 (CDR-H2), and 92 (CDR-H3);
SEQ ID NOS: 21 (CDR-L1), 33 (CDR-L2), 52 (CDR-L3), 61 (CDR-H1), and 74 (CDR-H2);
SEQ ID NOS: 21 (CDR-L1), 33 (CDR-L2), 52 (CDR-L3), 101 (CDR-H1), and 111 (CDR-H2);
SEQ ID NOS: 21 (CDR-L1), 34 (CDR-L2), 53 (CDR-L3), 61 (CDR-H1), 75 (CDR-H2), and 93 (CDR-H3);
SEQ ID NOS: 21 (CDR-L1), 34 (CDR-L2), 53 (CDR-L3), 101 (CDR-H1), 112 (CDR-H2), and 93 (CDR-H3);
SEQ ID NOS: 21 (CDR-L1), 35 (CDR-L2), 54 (CDR-L3), 61 (CDR-H1), 76 (CDR-H2), and 94 (CDR-H3);
SEQ ID NOS: 21 (CDR-L1), 35 (CDR-L2), 54 (CDR-L3), 101 (CDR-H1), 113 (CDR-H2), and 94 (CDR-H3);
SEQ ID NOS: 21 (CDR-L1), 36 (CDR-L2), 55 (CDR-L3), 61 (CDR-H1), 77 (CDR-H2), and 95 (CDR-H3); and
SEQ ID NOS: 21 (CDR-L1), 36 (CDR-L2), 55 (CDR-L3), 101 (CDR-H1), 114 (CDR-H2), and 95 (CDR-H3); and
detecting binding between the one or more additional scFvs reactive with TAR DNA-binding protein 43 (TDP-43) disease variants, alpha-syn disease variants, or tau disease variants and the first and/or the second biological sample.

13. The method of claim 11, wherein the scFv reactive with one or more TDP-43 disease variants has the amino acid sequence set forth in one of SEQ ID NOs: 8-20.

14. The method of claim 11, wherein the one or more additional scFvs is reactive with one or more alpha-syn disease variants has the amino acid sequence set forth in SEQ ID NO: 1, SEQ ID NO: 2, or SEQ ID NO: 5.

15. The method of claim 11, wherein the one or more additional scFvs is reactive with one or more tau disease variants has the amino acid sequence set forth in SEQ ID NO: 6 or SEQ ID NO: 7.

16. The method of claim 10, wherein the biological sample is a serum sample.

17. A kit for detecting frontotemporal dementia (FTD), the kit comprising:
a single-chain variable fragment (scFv) reactive with one or more oligomeric beta amyloid disease variants, wherein the scFv reactive with one or more oligomeric beta amyloid disease variants has a light chain complementary determining region (CDR) and heavy chain CDR combination selected from the group consisting of:
SEQ ID NOS: 21 (CDR-L1), 26 (CDR-L2), 43 (CDR-L3), 62 (CDR-H1), 66 (CDR-H2), and 85 (CDR-H3); and
SEQ ID NOS: 21 (CDR-L1), 27 (CDR-L2), 44 (CDR-L3), 61 (CDR-H1), 67 (CDR-H2), and 85 (CDR-H3);
one or more controls; and
instructions for using the kit.

18. The kit of claim 17, wherein the scFv reactive with one or more oligomeric beta amyloid disease variants has the amino acid sequence set forth in SEQ ID NO: 3 or SEQ ID NO: 4.

19. The kit of claim 17, wherein the instructions for using the kit comprise detecting binding between one or more scFvs and a biological sample from a subject, the binding being indicative of the subject having or likely to develop FTD.

20. The kit of claim 17, further comprising one or more additional scFvs reactive with TAR DNA-binding protein 43 (TDP-43) disease variants, alpha-syn disease variants, or tau disease variants, wherein the one or more additional scFvs reactive with TDP-43 disease variants, alpha-syn disease variants, or tau disease variants has a light chain CDR and heavy chain CDR combination or a light chain CDR combination and truncated heavy chain selected from the group consisting of:
SEQ ID NOS: 21 (CDR-L1), 24 (CDR-L2), 41 (CDR-L3), 61 (CDR-H1), 64 (CDR-H2), and 83 (CDR-H3);
SEQ ID NOS: 21 (CDR-L1), 24 (CDR-L2), 41 (CDR-L3), 101 (CDR-H1), 103 (CDR-H2), and 83 (CDR-H3);
SEQ ID NOS: 21 (CDR-L1), 25 (CDR-L2), 42 (CDR-L3), 61 (CDR-H1), 65 (CDR-H2), and 84 (CDR-H3);
SEQ ID NOS: 21 (CDR-L1), 28 (CDR-L2), 45 (CDR-L3), 61 (CDR-H1), 68 (CDR-H2), and 87 (CDR-H3);
SEQ ID NOS: 21 (CDR-L1), 28 (CDR-L2), 45 (CDR-L3), 101 (CDR-H1), 106 (CDR-H2), and 87 (CDR-H3);
SEQ ID NOS: 22 (CDR-L1), 29 (CDR-L2), 46 (CDR-L3), 61 (CDR-H1), 69 (CDR-H2), and 88 (CDR-H3);
SEQ ID NOS: 22 (CDR-L1), 29 (CDR-L2), 46 (CDR-L3), 101 (CDR-H1), 107 (CDR-H2), and 88 (CDR-H3);
SEQ ID NOS: 23 (CDR-L1), 30 (CDR-L2), 47 (CDR-L3), 63 (CDR-H1), 70 (CDR-H2), and 89 (CDR-H3);
SEQ ID NOS: 23 (CDR-L1), 30 (CDR-L2), 47 (CDR-L3), 102 (CDR-H1), 108 (CDR-H2), and 89 (CDR-H3);
SEQ ID NOS: 21 (CDR-L1), 32 (CDR-L2), and 49 (CDR-L3);
SEQ ID NOS: 21 (CDR-L1), 27 (CDR-L2), 50 (CDR-L3), 61 (CDR-H1), 72 (CDR-H2), and 91 (CDR-H3);
SEQ ID NOS: 21 (CDR-L1), 26 (CDR-L2), 51 (CDR-L3), 61 (CDR-H1), 73 (CDR-H2), and 92 (CDR-H3);
SEQ ID NOS: 21 (CDR-L1), 26 (CDR-L2), 51 (CDR-L3), 101 (CDR-H1), 110 (CDR-H2), and 92 (CDR-H3);
SEQ ID NOS: 21 (CDR-L1), 33 (CDR-L2), 52 (CDR-L3), 61 (CDR-H1), and 74 (CDR-H2);
SEQ ID NOS: 21 (CDR-L1), 33 (CDR-L2), 52 (CDR-L3), 101 (CDR-H1), and 111 (CDR-H2);
SEQ ID NOS: 21 (CDR-L1), 34 (CDR-L2), 53 (CDR-L3), 61 (CDR-H1), 75 (CDR-H2), and 93 (CDR-H3);
SEQ ID NOS: 21 (CDR-L1), 34 (CDR-L2), 53 (CDR-L3), 101 (CDR-H1), 112 (CDR-H2), and 93 (CDR-H3);
SEQ ID NOS: 21 (CDR-L1), 35 (CDR-L2), 54 (CDR-L3), 61 (CDR-H1), 76 (CDR-H2), and 94 (CDR-H3);
SEQ ID NOS: 21 (CDR-L1), 35 (CDR-L2), 54 (CDR-L3), 101 (CDR-H1), 113 (CDR-H2), and 94 (CDR-H3);
SEQ ID NOS: 21 (CDR-L1), 36 (CDR-L2), 55 (CDR-L3), 61 (CDR-H1), 77 (CDR-H2), and 95 (CDR-H3); and
SEQ ID NOS: 21 (CDR-L1), 36 (CDR-L2), 55 (CDR-L3), 101 (CDR-H1), 114 (CDR-H2), and 95 (CDR-H3).
